(19) United States
(12) Patent Application Publication
(10) Pub. No.: US 2005/0282840 A1
(43) Pub. Date: Dec. 22, 2005

(54) Methods of modulating neurotrophin-mediated activity

(75) Inventors: Gregory M. Ross, Sudbury (CA); Walter A. Szarek, Kingston (CA); Rahul Vohra, Kanata (CA)

Correspondence Address: LAHIVE & COCKFIELD, LLP., 28 STATE STREET, BOSTON, MA, 02109, US

(73) Assignees: PainCeptor Pharma Corporation, St. Laurent (CA); Queen's University at Kingston, Kingston (CA)

(21) Appl. No.: 11/057,084

(22) Filed: Feb. 11, 2005

Related U.S. Application Data
(60) Provisional application No. 60/544,267, filed on Feb. 11, 2004.
(60) Provisional application No. 60/564,106, filed on Apr. 20, 2004.

(51) Int. Cl. A61K 31/513; A61K 31/42

(52) U.S. Cl. 514/269; 514/378

(57) ABSTRACT

Disclosed are compositions which modulate the interaction of nerve growth factor and brain-derived neurotrophic factor with neurotrophic receptors. Also disclosed are methods of using the compositions of the invention, including methods of administration.

### RELATED APPLICATIONS

This application claims priority to U.S. Provisional Application No. 60/544,267, Attorney Docket No. PCI-004-1, filed Feb. 11, 2004, entitled “METHODS OF MODULATING NEUROTROPHIN-MEDIATED ACTIVITY” and U.S. Provisional Application No. 60/564,106, Attorney Docket No. PCI-004-2, filed Apr. 20, 2004, entitled “METHODS OF MODULATING NEUROTROPHIN-MEDIATED ACTIVITY.” The entire contents of each of the aforementioned applications are hereby expressly incorporated herein by reference in their entireties.

### TECHNICAL FIELD

The present invention relates to compositions which modulate the interaction of nerve growth factor and brain-derived neurotrophic factor with their respective receptors TrkA and TrkB, as well as the common neurotrophin receptor p75^{NTR}, and methods of use thereof.

### BACKGROUND

The neurotrophins are a family of structurally and functionally related proteins, including Nerve Growth Factor (NGF), Brain-Derived Neurotrophic Factor (BDNF), Neurotrophin-3 (NT-3), Neurotrophin-4/5 (NT-4/5) and Neurotrophin-6 (NT-6). These proteins promote the survival and differentiation of diverse neuronal populations in both the peripheral and central nervous systems and are involved in the pathogenesis of diverse neurological disorders (Hefti, J. Neurosci. 6:2155-2162 (1986); Hefti and Weiner, Annals of Neurology 20:275-281 (1986); Levi-Montalcini, EMBO J. 6:1145-1154 (1987); Barde, Neuron 2:1525-1534 (1989); Leibrock et al., Nature 341:149-152 (1989); Maisonpierre et al., Science 247:1446-1451 (1990); Rosenthal et al., Neuron 4:767-773 (1990); Hohn et al., Nature 344:339-341 (1990); Gotz et al., Nature 372:266-269 (1994); Maness et al., Neurosci. Biobehav. Rev. 18:143-159 (1994); Dechant et al., Nature Neurosci. 5:1131-1136 (2002). This broad spectrum of biological activities exerted by the neurotrophins results from their ability to bind and activate two structurally unrelated receptor types, the p75 neurotrophin receptor (p75^{NTR}) and the three members of the Trk receptor family of tyrosine kinases (Kaplan et al., Curr. Opin. Cell Biol. 9:213-221 (1997); Friedman et al., Exp. Cell Res. 253:131-142 (1999); Patapoutian et al., Curr. Opin. Neurobiol. 11:272-280 (2001).

The common neurotrophin receptor p75^{NTR} is a transmembrane glycoprotein structurally related to the tumor necrosis factor and CD-40 receptors (Meakin and Shooter, Trends Neurosci. 15:323-331 (1992 Rydén and Ibáñez, J. Biol. Chem. 271:5623-5627 (1996). As all neurotrophins bind to p75^{NTR} with similar affinity (Rodrigues-Tébar et al., Neuron 4:487-492 (1990); Hallbook et al., Neuron 6:845-858 (1991); Rodrigues-Tébar et al., EMBO J. 11:917-922 (1992); Ibáñez, Trends Biotech. 13:217-227 (1995)), neurotrophin specificity is conventionally thought to be caused by the binding selectivity for Trk receptors which are differentially expressed in different neuronal populations (Ibáñez, Trends Biotech. 13:217-227 (1995)). However, accumulated experimental data on neurotrophin activity reveal important functional aspects of p75^{NTR} (Heldin et al., J. Biol. Chem. 264:8905-8912 (1989); Jing et al., Neuron 9:1067-1079 (1992); Herrmann et al., Mol. Biol. 4:1205-1216 (1993); Barker and Shooter, Neuron 13:203-215 (1994); Dobrowsky et al., Science 265:1596-1599 (1994), Matsumoto et al., Cancer Res. 55:1798-1806 (1995); Marchetti et al., Cancer Res. 56:2856-2863 (1996); Washiyama et al., Amer. J. Path. 148:929-940 (1996)). The common neurotrophin receptor enhances functions and increases binding specificity of Trk receptors (Barker and Shooter, Neuron 13:203-215 (1994); Mahadeo et al., J. Biol. Chem. 269:6884-6891 (1994); Chao and Hempstead, Trends Neurosci. 18:321-326 (1995); Rydén and Ibáñez, J. Biol. Chem. 271:5623-5627 (1996)). In addition, p75^{NTR} possesses unique, neurotrophin dependent, Trk-independent signaling properties which involve ceramide production through activation of the sphingomyelin cycle (Dobrowsky et al., Science 265:1596-1599 (1994)) apoptosis (cell death) (Cassacia-Bonnefil et al., Nature 383:716-719 (1996)), and activation of the transcription factor NFKB (Carter et al., Science 272:542-545 (1996)).

Moreover, while initially studied primarily in neurons, p75^{NTR} has also been found to play critical roles in vascular biology (von Schack et al., Nat. Neurosci. 4:977-978, 2001; Wan et al., Am. J. Pathol. 157:1247-1258, 2001 ), glial biology (Bentley et al., J. Neurosci. 20:7706-7715, 2000; Syroid et al., J. Neurosci. 20:5741-5747, 2000), the immune system (Tokuoka et al., Br. J. Pharmacol. 134:1580-1586, 2001), and tumor biology (Sakamoti et al., Oncol. Rep. 8:973-980, 2001; Descamps et al., J. Biol. Chem. 276:17864017870, 2001). For example, p75^{NTR} has been demonstrated to participate in human melanoma progression (Herrmann et al., Mol. Biol. 4:1205-1216 (1993); Marchetti et al., Cancer Res. 56:2856-2863 (1996)). Furthermore, NGF and NT-3 increase the production of heparin by 70W melanoma cells, which is associated with their metastatic potential (Marchetti et al., Cancer Res. 56:2856-2863 (1996)).

Unlike p75^{NTR}, the Trk receptors (TrkA, TrkB and TrkC) exhibit selectivity for specific neurotrophins. (Kaplan et al., 1991; Klein et al., 1991,1992; Soppet et al., 1991; Squinto et al., 1991; Berkemeier et al., 1991; Escandon et al., 1993; Lamballe et al., 1991). For example, TrkA primarily binds NGF (Kaplan et al., 1991; Klein et al., 1991) and has been reported to bind NT-3 (J. Biol. Chem. 271(10):5623-7, 1996); TrkB binds BDNF and NT-4/5 (Soppet et al., 1991; Squinto et al., 1991; Berkemeier et al., 1991; Escandon et al., 1993; Lamballe et al., 1991; Klein et al., 1992; Vale and Shooter, 1985; Barbacid, 1993); and TrkC exclusively binds NT-3 (Lamballe et al., 1991; Vale and Shooter, 1985). This is particularly evident when the Trk receptors are coexpressed with the common neurotrophin receptor p75^{NTR}. (For review see Meakin and Shooter, 1992; Barbacid, 1993; Chao, 1994; Bradshaw et al., 1994; Ibáñez, 1995).

Biochemical experiments indicate that neurotrophin receptors form three different types of complexes: homodimers of Trk receptors, homomeric p75^{NTR} receptors and mixed complexes of both Trk and p75^{NTR}. These complexes may coexist in cells and may be linked through biochemical equilibria. Functionally, their signaling can be independent, synergistic or antagonistic. The response of a cell to neurotrophins is thus determined by the quantitative and qualitative composition of its receptor complement in combination with biochemical equilibria between pools of active and inactive receptors (Dechant, Cell Tissue Res. 305:229-238, 2001), as well as other cellular and biochemical components downstream of the neurotrophin receptors, e.g., the availability of proteins, lipids and inorganic molecules involved in signal transcution.

Due to the implication of neurotrophin binding to homomeric and heteromeric neurotrophin receptor complexes in various disease states, a need exists for pharmaceutical agents and methods of use thereof for modulating the interactions of neurotrophins with the common neurotrophin receptors, e.g., p75^{NTR}, the Trk receptors (TrkA, TrkB and TrkC), etc.

### SUMMARY OF THE INVENTION

In one aspect, the invention provides a method of modulating the interaction of a neurotrophin and a neurotrophin receptor, comprising contacting cells expressing a neurotrophin receptor with an effective amount of a compound of Formula 1, 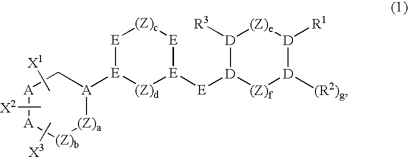
wherein A, E and D are each, independently, an sp²- or sp³-hybridized oxygen, carbon, nitrogen, or sulfur atom; X¹, X² and X³ are each, independently, selected from the group consisting of a hydrogen atom, C₁-C₆-alkyl, an electronegative atom, an electronegative functional group, and N(R⁴)R⁵, wherein R⁴ and R⁵ are each, independently, selected from the group consisting of H, aryl, and C₁-C₆-alkyl; esters thereof, salts thereof, and any combination thereof; R¹, R² and R³ are each, independently, selected from the group consisting of a hydrogen atom, C₁-C₆-alkyl, an electronegative atom, and N(R⁴)R⁵, wherein R⁴ and R⁵ are each, independently, selected from the group consisting of H, aryl, and C₁-C₆-alkyl; esters thereof, salts thereof, and any combination thereof; Z is independently selected from the group consisting of C═S, C, O, S, CH, C(O), N, NH, C-alkyl, N-alkyl, C-aryl, N-aryl, N-cycloalkyl and N—P, where P is a carbohydrate moiety; a, b, c, d, e, f and g are each 0 or 1, provided that at least one of a and b is 1, at least one of c and d is 1, and at least one of e and f is 1.

In another aspect, the invention provides a method of modulating the interaction of a neurotrophin and a neurotrophin receptor, comprising contacting cells expressing a neurotrophin receptor with an effective amount of a compound of Formula 2, 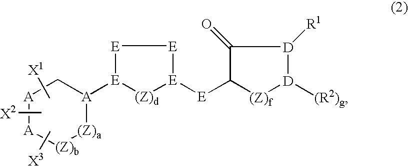
wherein A, E and D are each, independently, an sp²- or sp³-hybridized carbon, nitrogen, oxygen or sulfur atom; X¹, X² and X³ are each, independently, selected from the group consisting of a hydrogen atom, C₁-C₆-alkyl, an electronegative atom, an electronegative functional group, and N(R⁴)R⁵, where R⁴ and R⁵ are each, independently, H, aryl, and C₁-C₆-alkyl; esters thereof, salts thereof, and any combination thereof; R¹ and R² are each, independently, selected from the group consisting of a hydrogen atom, C₁-C₆-alkyl, an electronegative atom, an electronegative functional group, and N(R⁴)R⁵, wherein R⁴ and R⁵ are each, independently, selected from the group consisting of H, aryl, and C₁-C₆-alkyl; esters thereof, salts thereof, and any combination thereof; Z is independently selected from the group consisting of C═S, C, O, S, CH, C(O), N, NH, C-alkyl, N-alkyl, C-aryl, N-aryl, N-cycloalkyl and N—P; a, b and g are each 0 or 1, provided that at least one of a and b is 1, and d and f are each 1.

In yet another aspect, the invention provides a method of modulating the interaction of a neurotrophin and a neurotrophin receptor, comprising contacting cells expressing a neurotrophin receptor with an effective amount of a compound of Formula 3, 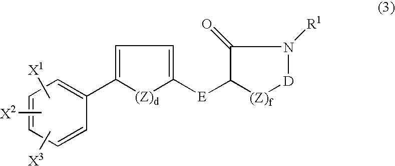
wherein E and D are each, independently, an sp²- or sp³-hybridized carbon, nitrogen, oxygen or sulfur atom; X¹, X² and X³ are each, independently, selected from the group consisting of a hydrogen atom, C₁-C₆-alkyl, an electronegative atom, an electronegative functional group and N(R⁴)R⁵, wherein R⁴ and R⁵ are each, independently, selected from the group consisting of H, aryl, and C₁-C₆-alkyl; esters thereof, salts thereof, and any combination thereof; R¹ is selected from the group consisting of a hydrogen atom, C₁-C₆-alkyl, an electronegative atom, an electronegative functional group and N(R⁴)R⁵, wherein R⁴ and R⁵ are each, independently, selected from the group consisting of H, aryl, and C₁-C₆-alkyl; esters thereof, salts thereof, and any combination thereof; Z is independently selected from the group consisting of C═S, C, O, S, CH, C(O), N, NH, C-alkyl, N-alkyl, C-aryl, N-aryl, N-cycloalkyl and N—P; d and f are each 1.

In another aspect, the invention provides a method of modulating the interaction of a neurotrophin and a neurotrophin receptor, comprising contacting cells expressing a neurotrophin receptor with an effective amount of a compound of Formula 4, 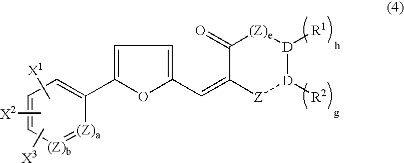
wherein the dashed line indicates a double or single bond;
each D is, independently, an sp²- or sp³-hybridized carbon, nitrogen, oxygen or sulfur atom; X¹, X² and X³ are each, independently, a hydrogen atom, C₁-C₆-alkyl, an electronegative atom or an electronegative functional group or N(R⁴)R⁵, wherein R⁴ and R⁵ are each, independently, H, aryl, C₁-C₆-alkyl, esters thereof, salts thereof, and any combination thereof; R¹ and R² are each, independently, a hydrogen atom, C₁-C₆-alkyl, an electronegative atom or an electronegative functional group, or N(R⁴)R⁵, where R⁴ and R⁵ are each, independently, H, aryl, or C₁-C₆-alkyl; esters thereof, salts thereof, and any combination thereof; Z is independently selected from the group consisting of C═S, C, O, S, CH, C(O), N, NH, C-alkyl, N-alkyl, C-aryl, N-aryl, N-cycloalkyl and N—P, wherein P is a carbohydrate moiety; and a, b and e are each 0 or 1. In one embodiment of formula 4, X¹, X² and X³ are each, independently, selected from the group consisting of a hydrogen atom, C₁-C₆-alkyl, —OH, —CO₂H, —CO₂C₁-C₆, —[(CH₂)₁₋₆]CO₂H, aryl-CO₂H, aryl-CO₂C₁-C₆, —O[(CH₂)₀₋₆]CH₃, —SO₃H, —SO₂H, —SO₂NH₂, —PO₃H₂, —NO₂, —ONO₂, —CNO, —OSO₃H, —OC(O)(OH), O[(CH₂)₁₋₆]CO₂H, and —CO₂(CH₂)₁₋₆H; R¹ and R² are each, independently, selected from the group consisting of a hydrogen atom, C₁-C₆-alkyl, an electronegative atom and an electronegative functional group; Z is independently selected from the group consisting of C═S, C, O, S, CH, C(O), N, NH, C-alkyl, N-alkyl, C-aryl, N-aryl, and N-cycloalkyl; and a and b are 1 and e, g and h are each 0 or 1. In yet another embodiment of formula 4, X¹, X² and X³ are each, independently, selected from the group consisting of a hydrogen atom, C₁-C₆-alkyl, —OH, —CO₂H, —CO₂C₁-C₆, —[(CH₂)₁₋₆]CO₂H, —SO₃H, —SO₂H, —SO₂NH₂, —PO₃H₂, —NO₂, —O[(CH₂)₁₋₆]CO₂H, and —CO₂(CH₂)₁₋₆H; R¹ and R² are each, independently, selected from the group consisting of a hydrogen atom, C₁-C₆-alkyl, —OH, —CN, —CO₂H, —CO₂C₁-C₆, —[(CH₂)₁₋₆]CO₂H, —[(CH₂)₁₋₆]CO₂(CH₂)₀₋₆CH₃, —C₁-C₆—COO—C₁-C₆, halomethyl, dihalomethyl, and trihalomethyl; Z is independently selected from the group consisting of C═S, S, CH, C(O), N, NH, C-alkyl, N-alkyl, C-aryl, N-aryl, and N-cycloalkyl; and a and b are 1 and e is 0or 1.

In another aspect, the invention provides a method of modulating the interaction of a neurotrophin and a neurotrophin receptor, comprising contacting cells expressing a neurotrophin receptor with an effective amount of a compound of Formula 5, 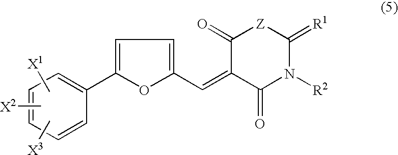
wherein X¹, X² and X³ are each, independently, selected from the group consisting of a hydrogen atom, C₁-C₆-alkyl, an electronegative atom, an electronegative functional group, and N(R⁴)R⁵, wherein R⁴ and R⁵ are each, independently, H, aryl, C₁-C₆-alkyl; esters thereof, salts thereof, and any combination thereof; R¹ and R² are each, independently, selected from the group consisting of a hydrogen atom, C₁-C₆-alkyl, an electronegative atom, an electronegative functional group, and N(R⁴)R⁵, where R⁴ and R⁵ are each, independently, H, aryl, or C₁-C₆-alkyl; esters thereof, salts thereof, and any combination thereof, Z is independently selected from the group consisting of C═S, C, O, S, CH, C(O), N, NH, C-alkyl, N-alkyl, C-aryl, N-aryl, N-cycloalkyl and N—P, where P is a carbohydrate moiety.

In one embodiment of formula 5, X¹, X² and X³ are each, independently, selected from the group consisting of a hydrogen atom, C₁-C₆-alkyl, —OH, —CO₂H, —CO₂C₁-C₆, —[(CH₂)₁₋₆]CO₂H, aryl-CO₂H, aryl-CO₂C₁-C₆, —O[(CH₂)₀₋₆]CH₃, —SO₃H, —SO₂H, —SO₂NH₂, —PO₃H₂, —NO₂, —ONO₂, —CNO, —OSO₃H, —OC(O)(OH), O[(CH₂)₁₋₆]CO₂H, and —CO₂(CH₂)₁₋₆H; R¹ and R² are each, independently, selected from the group consisting of a hydrogen atom, C₁-C₆-alkyl, an electronegative atom or an electronegative functional group; Z is independently selected from the group consisting of C═S, C, O, S, CH, C(O), N, NH, C-alkyl, N-alkyl, C-aryl, N-aryl, and N-cycloalkyl.

In another embodiment of formula 5, X¹, X² and X³ are each, independently, selected from the group consisting of a hydrogen atom, C₁-C₆-alkyl, —OH; —CO₂H, —CO₂C₁-C₆, —[(CH₂)₁₋₆]CO₂H, —SO₃H, —SO₂H, —SO₂NH₂, —PO₃H₂, —NO₂, O[(CH₂)₁₋₆]CO₂H, and —CO₂(CH₂)₁₋₆H; R¹ is O or S; R² is, independently, selected from the group consisting of a hydrogen atom, C₁-C₆-alkyl, —OH, —CN, —CO₂H, —CO₂C₁-C₆, —[(CH₂)₁₋₆]CO₂H, —[(CH₂)₁₋₆]CO₂(CH₂)₀₋₆CH₃, —C₁-C₆—COO—C₁-C₆, halomethyl, dihalomethyl, and trihalomethyl; and Z is independently selected from the group consisting of C═S, S, CH, C(O), N, NH, C-alkyl, N-alkyl, C-aryl, N-aryl, or N-cycloalkyl.

In another aspect, the invention provides a method of modulating the interaction of a neurotrophin and a neurotrophin receptor, comprising contacting cells expressing a neurotrophin receptor with an effective amount of a compound of Formula 6, 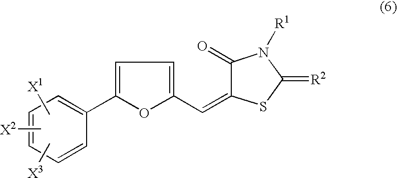
wherein X¹, X² and X³ are each, independently, selected from the group consisting of a hydrogen atom, C₁-C₆-alkyl, an electronegative atom, an electronegative functional group, and N(R⁴)R⁵, wherein R⁴ and R⁵ are each, independently, selected from the group consisting of H, aryl, and C₁-C₆-alkyl; esters thereof, salts thereof, and any combination thereof; R¹ and R² are each, independently, selected from the group consisting of a hydrogen atom, C₁-C₆-alkyl, an electronegative atom, an electronegative functional group and N(R⁴)R⁵, wherein R⁴ and R⁵ are each, independently, selected from the group consisting of H, aryl, and C₁-C₆-alkyl; esters thereof, salts thereof, and any combination thereof; and Z is independently selected from the group consisting of C═S, C, O, S, CH, C(O), N, NH, C-alkyl, N-alkyl, C-aryl, N-aryl, N-cycloalkyl and N—P, wherein P is a carbohydrate moiety.

In one embodiment of formula 6, X¹, X² and X³ are each, independently, selected from the group consisting of a hydrogen atom, C₁-C₆-alkyl, —OH; —CO₂H, —CO₂C₁-C₆, —[(CH₂)₁₋₆]CO₂H, aryl-CO₂H, aryl-CO₂C₁-C₆, —O[(CH₂)₀₋₆]CH₃, —SO₃H, —SO₂H, —SO₂NH₂, —PO₃H₂, —NO₂, —ONO₂, —CNO, —OSO₃H, —OC(O)(OH), O[(CH₂)₁₋₆]CO₂H, —CO₂(CH₂)₁₋₆H; and R¹ and R² are each, independently, selected from the group consisting of a hydrogen atom, C₁-C₆-alkyl, an electronegative atom and an electronegative functional group; and Z is independently selected from the group consisting of C═S, C, O, S, CH, C(O), N, NH, C-alkyl, N-alkyl, C-aryl, N-aryl, and N-cycloalkyl.

In another embodiment of formula 6, X¹, X² and X³ are each, independently, selected from the group consisting of a hydrogen atom, C₁-C₆-alkyl, —OH; —CO₂H; —CO₂C₁-C₆, —[(CH₂)₁₋₆]CO₂H, —SO₃H; —SO₂H; —SO₂NH₂, —PO₃H₂; —NO₂; O[(CH₂)₁₋₆]CO₂H, or —CO₂(CH₂)₁₋₆H; R¹ is selected from the group consisting of a hydrogen atom, C₁-C₆-alkyl, —OH; —CN; —CO₂H; —CO₂C₁-C₆, —[(CH₂)₁₋₆]CO₂H, —[(CH₂)₁₋₆]CO₂(CH₂)₀₋₆CH₃, —C₁-C₆—COO—C₁-C₆, halomethyl, dihalomethyl, and trihalomethyl; R² is O or S; and Z is independently selected from the group consisting of C═S, S, CH, or C(O), N, NH, C-alkyl, N-alkyl, C-aryl, N-aryl, and N-cycloalkyl. In one embodiment of the invention, at least one of X¹, X² and X³ of Formulas 1, 2, 3, 4, 5, and 6 is an electronegative atom or electronegative functional group. In another embodiment, at least two of X¹, X² and X³ of Formulas 1, 2, 3, 4, 5, and 6 are either an electronegative atom or electronegative functional group.

In related embodiments of the invention, at least one of R¹, R² and R³ of Formulas 1, 2, 3, 4, 5, and 6 is an electronegative atom or electronegative functional group. In another aspect, at least two of R¹, R² and R³ of Formulas 1, 2, 3, 4, 5, and 6 are either an electronegative atom or electronegative functional group. In another embodiment of the invention, the neurotrophin is selected from the group consisting of nerve growth factor, brain-derived neurotrophic factor, neurotrophin-3, neurotrophin-4/5 and precursors thereof. In another embodiment, the neurotrophin receptor is selected from the group consisting of p75^{NTR}, TrkA, TrkB and TrkC.

In some embodiments of the invention, the neurotrophin is nerve growth factor or proNGF, and the neurotrophin receptor is p75^{NTR}. In other embodiments, the the neurotrophin is nerve growth factor or proNGF and the neurotrophin receptor is TrkA. In still other embodiments, the compound of Formulas 1, 2, 3, 4, 5, and 6 further modulates the interaction of NGF and/or proNGF with TrkA.

In other embodiments, the neurotrophin is brain-derived growth factor (BDNF) and/or proBDNF, and the neurotrophin receptor is p75^{NTR}. In other embodiments, the neurotrophin is BDNF and/or proBDNF, and the neurotrophin receptor is TrkB. In yet other embodiments, the compound further modulates the interaction of BDNF and/or proBDNF with TrkB.

In other embodiments, the compound of Formulas 1, 2, 3, 4, 5, and 6 further modulates the interaction of a second neurotrophin with a neurotrophin receptor. In some embodiments, the second neurotrophin is selected from the group consisting of nerve growth factor, brain-derived neurotrophic factor, neurotrophin-3, neurotrophin-4/5 and precursors thereof. In still other embodiments, the first neurotrophin is NGF and/or proNGF and the second neurotrophin is BDNF and/or proBDNF. In yet another embodiment, the neurotrophin receptor is p75^{NTR}. In other embodiments, the compound of Formulas 1, 2, 3, 4, 5, and 6 further modulates the interaction of NGF and/or proNGF with TrkA. In other embodiments, the compound Formula 1, 2, 3, 4, 5, and 6 further modulates the interaction of BDNF and/or proBDNF with TrkB.

In another embodiment, the compound of Formula 1, 2, 3, 4, 5, and 6 further modulates the interaction of the neurotrophin with TrkC.

In another aspect, the invention provides a method to modulate a neurotrophin-mediated activity in a subject in need thereof. In certain embodiments, the neurotrophin-mediated activity is associated with pain. In other embodiments, the neurotrophin-mediated activity is associated with an inflammatory disorder. In other embodiments, the neurotrophin-mediated activity is associated with a neurological disorder.

In certain embodiments, the pain is selected from the group consisting of cutaneous pain, somatic pain, visceral pain and neuropathic pain. In still another embodiment, the pain is acute pain or chronic pain. In another embodiment, the cutaneous pain is associated with injury, trauma, a cut, a laceration, a bum, a surgical incision, an infection or acute inflammation. In yet another embodiment, the somatic pain is associated with an injury, disease or disorder of the musculoskeletal and connective system or the circulatory system. In another embodiment, the injury disease or disorder is selected from the group consisting of sprains, broken bones, arthritis, psoriasis, eczema, and ischemic heart disease.

In another embodiment, the visceral pain is associated with an injury, disease or disorder of the circulatory system, the respiratory system, the gastrointestinal system, the genitourinary system or the immune system. In another embodiment, the disease or disorder of the circulatory system is selected from the group consisting of ischaemic heart disease, angina, acute myocardial infarction, cardiac arrhythmia, phlebitis, varicose veins, intermittent claudication, and hemorrhoids. In another embodiment, the disease or disorder of the respiratory system is selected from the group consisting of asthma, respiratory infection, chronic bronchitis and emphysema. In another embodiment, the disease or disorder of the gastrointestinal system is selected from the group consisting of gastritis, duodenitis, irritable bowel syndrome, colitis, Crohn's disease, gastrointestinal reflux disease, ulcers and diverticulitis. In still another embodiment, the disease or disorder of the genitourinary system is selected from the group consisting of cystitis, urinary tract infections, glomuerulonephritis, polycystic kidney disease, kidney stones and cancers of the genitourinary system. In another embodiment, the somatic pain is selected from the group consisting of arthralgia, myalgia, chronic lower back pain, phantom limb pain, cancer-associated pain, dental pain, fibromyalgia, idiopathic pain disorder, chronic non-specific pain, chronic pelvic pain, post-operative pain, and referred pain. In yet another embodiment, the neuropathic pain is associated with an injury, disease or disorder of the nervous system. In another embodiment, the injury, disease or disorder of the nervous system is selected from the group consisting of neuralgia, neuropathy, headache, psychogenic pain, chronic cephalic pain and spinal cord injury.

In still another embodiment, the neurotrophin-mediated activity is selected from an inflammatory disorder of the musculoskeletal and connective tissue system, the respiratory system, the circulatory system, the genitourinary system, the gastrointestinal system or the nervous system. In yet another embodiment, the inflammatory disorder of the musculoskeletal and connective tissue system is selected from the group consisting of arthritis, psoriasis, myocitis, dermatitis and eczema. In another embodiment, the inflammatory disorder of the respiratory system is selected from the group consisting of asthma, bronchitis, sinusitis, pharyngitis, laryngitis, tracheitis, rhinitis, cystic fibrosis, respiratory infection and acute respiratory distress syndrome. In yet another embodiment, the inflammatory disorder of the circulatory system is selected from the group consisting of vasculitis, haematuria syndrome, artherosclerosis, arteritis, phlebitis, carditis and coronary heart disease. In another aspect, the inflammatory disorder of the gastrointestinal system is selected from the group consisting of inflammatory bowel disorder, ulcerative colitis, Crohn's disease, diverticulitis, viral infection, bacterial infection, peptic ulcer, chronic hepatitis, gingivitis, periodentitis, stomatitis, gastritis and gastrointestinal reflux disease. In another embodiment, the inflammatory disorder of the genitourinary system is selected from the group consisting of cystitis, polycystic kidney disease, nephritic syndrome, urinary tract infection, cystinosis, prostatitis, salpingitis, endometriosis and genitourinary cancer.

In yet another embodiment, the neurological disorder is selected from the group consisting of schizophrenia, bipolar disorder, depression, Alzheimer's disease, epilepsy, multiple sclerosis, amyotrophic lateral sclerosis, stroke, addiction, cerebral ischemia, neuropathy, retinal pigment degeneration, glaucoma, cardiac arrhythmia, Huntington's chorea, and Parkinson disease.

In another aspect, the invention provides a method of treating pain in a subject in need thereof, comprising administering to the subject an effective amount of a compound of Formula 1, Formula 2, Formula 3, Formula 4, Formula 5 and Formula 6.

In one embodiment, the pain is selected from the group consisting of cutaneous pain, somatic pain, visceral pain and neuropathic pain. In another embodiment, the pain is acute pain or chronic pain.

In another aspect, the invention provides a method of treating an inflammatory disorder in a subject in need thereof, comprising administering to the subject and effective amount of a compound of Formula 1, Formula 2, Formula 3, Formula 4, Formula 5 and Formula 6. In one embodiment, the inflammatory disorder is inflammatory disorder of the musculoskeletal and connective tissue system, the respiratory system, the circulatory system, the genitourinary system, the gastrointestinal system or the nervous system.

In another aspect, the invention provides a method of treating a neurological disorder in a subject in need thereof, comprising administering an effective amount of a compound of Formula 1, Formula 2, Formula 3, Formula 4, Formula 5 and Formula 6. In one embodiment, the neurological disorder is selected from the group consisting of schizophrenia, bipolar disorder, depression, Alzheimer's disease, epilepsy, multiple sclerosis, amyotrophic lateral sclerosis, stroke, addiction, cerebral ischemia, neuropathy, retinal pigment degeneration, glaucoma, cardiac arrhythmia, Huntington's chorea, and Parkinson disease. In another aspect, the invention provides any of the previous methods, further comprising administering to the subject an additional therapeutic agent. In one embodiment, the additional therapeutic agent is selected from the group consisting of an analgesic, an anti-inflammatory agent, an anesthetic, a corticosteroid, an anti-convulsant, an antidepressant, an anti-nausea agent, an anti-psychiatric agent, a cardiovascular agent and a cancer therapeutic.

In another aspect, the invention provides a pharmaceutical composition comprising a compound of Formula 1, Formula 2, Formula 3, Formula 4, Formula 5 and Formula 6.

### BRIEF DESCRIPTION OF THE DRAWINGS

FIG. 1A is a line graph depicting the inhibition of ¹²⁵I-NGF crosslinking to p75^{NTR} 5 receptors on PC12 cells in the presence of compound A or compound B. FIG. 1B is a line graph depicting the inhibition of ¹²⁵I-NGF crosslinking to TrkA receptors on PC12 cells in the presence of compound B. FIG. 1C is a bar graph depicting the inhibition of ¹²⁵I-NGF crosslinking to TrkA receptors on PC12 cells in the presence of compound A. FIGS. 1D and 1E depict the structures of compounds A and B, respectively.

FIG. 2A is a line graph depicting the inhibition of ¹²⁵I-BDNF crosslinking to p75^{NTR} receptors on nnr5 cells expressing p75^{NTR} in the presence of compound A or compound B. FIG. 2B is a line graph depicting the inhibition of ¹²⁵I-BDNF crosslinking to TrkB receptors on nnr5 cells expressing TrkB in the presence of compound A or compound B.

FIGS. 3A and 3B are SDS-PAGE gels demonstrating the results of the experiment described in Example 3.

### DETAILED DESCRIPTION OF THE INVENTION

The present invention relates to the discovery of compounds which modulate the interaction of a neurotrophin with a neurotrophin receptor, for example, the common neurotrophin receptor p75^{NTR} and/or a Trk receptor. Such compounds are of use, for example, for modulating the interaction of NGF and/or BDNF and/or a precursor thereof (e.g., proNGF, proBDNF) to p75^{NTR}, and the compounds within the invention can also have the ability to modulate the interaction of NGF and/or proNGF with TrkA and/or the interaction of BDNF and/or proBDNF with TrkB. For example, a compound that modulates the binding of NGF or proNGF to p75^{NTR} can further modulate the binding of the neurotrophin to TrkA. Such compounds can also be used to treat a subject having a condition having at least one symptom that is directly or indirectly mediated, at least in part, by the interaction of NGF and/or BDNF and/or a precursor thereof with p75^{NTR} and/or TrkA and TrkB respectively.

Nerve growth factor (also referred to hereinafter as “NGF”) is a prototypic neurotrophin, and is best known for its essential role during development of peripheral sensory and sympathetic neurons. NGF is produced as a high-molecular weight precursor (pro-NGF) that contains a pro-domain linked to the N-terminus which is cleaved by the endoprotease furin in the trans-Golgi network of neurons (Mowla et al., J. Biol. Chem. 276:12660-12666, 2001; Mowla et al., J. Neurosci. 19:2069-2080, 1999). Pro-NGF has been shown to be induced and secreted after injury to the CNS in an active form that is capable of triggering cell apoptosis (e.g., of neuronal cells and oligodendrocytes), and disruption of the interaction of pro-NGF and p75^{NTR} has been demonstrated to rescue injured adult rat corticospinal neurons (e.g., Harrington et al., PNAS USA 101(16):6226-6230, 2004). Mature NGF regulates the phenotype (e.g., cell body and dendrite size, gene expression and neurotransmitter phenotype) of peripheral neurons and certain CNS neurons, notably, basal forebrain and striatal cholinergic neurons throughout the life of an animal (Miller et al., Neuron 32:767-770, 2001; Ruberti et al., J. Neurosci. 20(7):2589, 2000; Chen et al, J. Neurosci. 17(19):7288-96, 1997; Fagan et al., J. Neurosci. 17(20):7644-54, 1997). NGF has been implicated in the pathogenesis of Alzheimer's disease, epilepsy and pain (Ben Ari and Represa, TINS 13:312-318 (1990); McKee et al., Ann. Neurol. 30:156 (1991); Leven and Mendel, TINS 16:353-359 (1993); Woolf and Doubell, Current Opinions in Neurobiol. 4:525-534 (1994); Rashid et al., Proc. Natl. Acad. Sci. U.S.A. 92:9495-9499 (1995); McMahon et al., Nature Med. 1:774-780 (1995)). The interaction of NGF with its receptors is determined by distinct sequences within its primary amino acid structure. While several regions of NGF participate in the NGF/TrkA interaction, mutation studies suggest that relatively few key residues, namely those located in the NGF amino and carboxyl termini, are primarily required for high affinity binding to TrkA.

NGF displays high and low affinity binding sites in sensory and sympathetic neurons and in pheochromocytoma PC12 cells (Sutter et al., J. Biol. Chem. 254:5972-5982 (1979); Landreth and Shooter, Proc. Natl. Acad. Sci. U.S.A. 77:4751-4755 (1980); Schechter and Bothwell, Cell 24:867-874 (1981)). The coexpression of the common neurotrophin p75^{NTR} receptor with TrkA is required to form the high affinity binding site (Hempstead et al., Nature 350:678-683 (1991); Barker and Shooter, Neuron 13:203-215 (1994); Mahadeo et al., J. Biol. Chem. 269:6884-6891 (1994); Chao and Hempstead, Trends Neurosci. 18:321-326 (1995)). Several models of the TrkA-p75^{NTR} interaction have been proposed to explain high affinity NGF binding (Bothwell, Cell 65:915-918 (1991); Chao, Neuron 9:583-593 (1992b); Chao and Hempstead, Trends Neurosci. 18:321-326 (1995); Wolf et al., J. Biol. Chem. 270:2133-2138 (1995); Ross et al., J. Cell Biol. 132:945-953 (1996); Ross et al., Nature Med. 3:872-878 (1997)). These models differ with respect to direct (conformational model) or indirect (ligand-presentation model) interaction of p75^{NTR} with TrkA. Direct TrkA-p75^{NTR} interaction is consistent with much of the existing experimental data.

Brain-derived neurotrophic factor (also referred to hereinafter as “BDNF”) supports neuronal survival and differentiation, and it also facilitates synaptic plasticity. BDNF was first shown to promote the outgrowth of spinal sensory neurons, but has since been shown to support the survival and outgrowth of sensory neurons, ganglion neurons, dopaminergic neurons, cholinergic neurons, GABAergic neurons and motor neurons.

In the sensory system, BDNF is produced by a subset of primary sensory neurons (nociceptors) that respond to tissue injury. The cell bodies of these biopolar neurons are located in the dorsal root ganglia (DRG). It is produced as a high molecular weight precursor (pro-BDNF) that contains a pro-domain linked to the N-terminus. Pro-BDNF is packaged in dense core vesicles (DCVs) where it is cleaved by the endoprotease PC1 to yield mature BDNF (Seidah et al. FEBS Lett. 379:247-250, 1996). After synthesis, BDNF is transported to the central terminals of sensory neurons in the dorsal horn where it can be released to interact with neurotrophin receptors p75^{NTR} and TrkB (Malcangio et al., Trends in Pharm. Sci. 24:116-121, 2003). Several lines of evidence indicate that BDNF acts within nociceptive circuitry and contributes to the development of exaggerated pain states following tissue or nerve injury within the spinal cord,. Exogenous BDNF applied to the DRG produces allodynia in animals (Zhou, XF et al. 2000. Eur J Neurosci 12:100-105.), and BDNF is upregulated in the DRG and dorsal horn in experimental models of inflammatory and neuropathic pain (Cho, H J et al. 1997. Brain Res 764:269-272.; Mannion, R J et al. 1999. Proc Natl Acad Sci USA 96:9385-9390.; Fukuoka, T, et al. 2001. J Neurosci 21:4891-4900).

The concentration of BDNF depends on the availability of NGF (Michael et al., J. Neurosci. 17:8476-8490, 1997). Therefore, the increase in NGF concentration in peripheral tissues that follows tissue injury (e.g., an inflammatory insult), enhances the expression of BDNF. Thus, like NGF, BDNF has been implicated in pain, and inflammation as well as neurological disorders (e.g., neuropsychiatric disorders, including schizophrenia, bipolar disorder, epilepsy and depression). For example, the BDNF concentration in nociceptors in models of chronic pain indicate that BDNF is involved in the dorsal horn mechanisms of segmental pain (Malcangio et al., supra). Furthermore, evidence suggests that BDNF is also relevant to a variety of neurodegenerative diseases such as Huntington's disease, Alzheimer's disease, depression, memory deficit and schizophrenia (Dawbarn D, Allen S J. 2003. Neuropathol Appl Neurobiol 29:211-230).

Definitions

The term “electronegative atom”, as used herein, refers to an atom which carries a partial or full negative charge in a particular compound under physiological conditions. The electronegative atom can be, for example, an oxygen atom, a nitrogen atom, a sulfur atom or a halogen atom, such as a fluorine, chlorine, bromine or iodine atom. Preferably the electronegative atom is an oxygen atom. The term “electronegative functional group”, as used herein, refers to a functional group which includes at least one electronegative atom. Electronegative groups include acid functional groups and other polar functional groups. For example, suitable electronegative functional groups include, but are not limited to, carbonyl, thiocarbonyl, ester, imino, amido, carboxylic acid, sulfonic acid, sulfinic acid, sulfamic acid, phosphonic acid, boronic acid, sulfate ester, hydroxyl, mercapto, cyano, cyanate, thiocyanate, isocyanate, isothiocyanate, carbonate, nitrate and nitro groups. It is to be understood that, unless otherwise indicated, reference herein to an acidic functional group also encompasses salts of that functional group in combination with a suitable cation.

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups.

The term alkyl further includes alkyl groups which can further include oxygen, nitrogen, sulfur or phosphorous atoms replacing one or more carbons of the hydrocarbon backbone. In an embodiment, a straight chain or branched chain alkyl has 10 or fewer carbon atoms in its backbone (e.g., C₁-C₁₀ for straight chain, C₃-C₁₀ for branched chain), and more preferably 6 or fewer. Likewise, preferred cycloalkyls have from 4-7 carbon atoms in their ring structure, and more preferably have 5 or 6 carbons in the ring structure.

The term “substituted” is intended to describe moieties having substituents replacing a hydrogen on one or more atoms, e.g. C or N, of a molecule. Such substituents can include, for example, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, morpholino, phenol, benzyl, phenyl, piperizine, cyclopentane, cyclohexane, pyridine, 5H-tetrazole, triazole, piperidine, or an aromatic or heteroaromatic moiety.

The term “aryl” includes groups, including 5- and 6-membered single-ring aromatic groups that may include from zero to four heteroatoms, for example, phenyl, pyrrole, furan, thiophene, thiazole, isothiaozole, imidazole, triazole, tetrazole, pyrazole, oxazole, isoxazole, pyridine, pyrazine, pyridazine, and pyrimidine, and the like. Furthermore, the term “aryl” includes multicyclic aryl groups, e.g., tricyclic, bicyclic, e.g., naphthalene, benzoxazole, benzodioxazole, benzothiazole, benzoimidazole, benzothiophene, methylenedioxyphenyl, quinoline, isoquinoline, anthryl, phenanthryl, napthridine, indole, benzofuran, purine, benzofuran, deazapurine, or indolizine. Those aryl groups having heteroatoms in the ring structure may also be referred to as “aryl heterocycles”, “heterocycles,” “heteroaryls” or “heteroaromatics.” The aromatic ring can be substituted at one or more ring positions with such substituents as described above, as for example, alkyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminoacarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The term “carbohydrate” includes compounds which can be factored into the formula Cₓ(H₂O)_{y}, such as a monosaccharide group, for example, a fucosyl, glucosyl, galactosyl, mannosyl, fructosyl, gulosyl, idosyl, talosyl, allosyl, altrosyl, ribosyl, arabinosyl, xylosyl or lyxosyl group. The carbohydrate can also be substituted with any of the groups described herein.

It will be noted that the structures of some of the compounds of this invention include asymmetric carbon atoms. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis. Furthermore, the structures and other compounds and moieties discussed in this application also include all tautomers thereof. Compounds described herein may be obtained though art recognized synthesis strategies.

Additionally, the phrase “any combination thereof” implies that any number of the listed functional groups and molecules may be combined to create a larger molecular architecture. For example, the terms “phenyl,” “carbonyl” (or “═O”), “—O—,” “—OH,” and C₁₋₆ (i. e., —CH₃ and CH₂CH₂CH₂—) can be combined to form a 3-methoxy-4-propoxybenzoic acid substituent. It is to be understood that when combining functional groups and molecules to create a larger molecular architecture, hydrogens can be removed or added, as required to satisfy the valence of each atom.

As used herein, the term “neurotrophic factor” or “neurotrophin” (also referred to herein as “NT”) refers to members of a family of proteins, usually in the form of dimers, that are structurally homologous to NGF. The term includes the high-molecular-weight precursors (pro-neurotrophins, e.g., pro-NGF, pro-BDNF) and the mature proteins which include three surface 3-hairpin loops, a p-strand, an internal reverse turn region, and N- and C-termini. Neurotrophins promote at least one of the biological activities related to vertebrate neuron survival, differentiation, and function, as determined using assays described, for example, in US 2002/0169182A1 and Riopelle et al., Can J. of Phys. and Pharm. 60:707 (1982); Harrington et al. PNAS USA 101(16):6226-6230, (2004)). Neurotrophic factors include, for example, brain-derived neurotrophic factor (BDNF), NGF, neurotrophin 3 (NT-3), neurotrophin 4/5 (NT-4/5), and neurotrophin 6 (NT-6) (R. M. Lindsay et al.: TINS, vol. 17, p. 182 (1994) and R. M. Lindsay: Phil. Trans. R. Soc. Lond. B. vol. 351, p. 365-373 (1996)). In addition, ciliary neurotrophic factor (CNTF), glia-derived neurotrophic factor (GDNF), glia growth factor (GGF2), central nerve growth factor (AF-1), hepatocyte growth factor (HGF) (A. Ebens et al., Neuron, vol. 17, p. 1157-1172 (1996)) can also be considered as neurotrophic factors. Moreover, biotechnologically engineered products of the above neurotrophic factors, which are derived by a partial substitution, an addition, a deletion or a removal by conventional genetic engineering techniques, are also included within the scope of the neurotrophic factors of the present invention as far as such product shows biological activities of the naturally-occurred neurotrophic factors.

As used herein, the term “neurotrophin receptor” (also referred to herein as “NTR”) is meant to refer to a receptor which binds a neurotrophin. In certain embodiments, the neurotrophin receptor is a member of the tyrosine kinase family of receptors, generally referred to as the “Trk” receptors or “Trks”, which are expressed on cellular surfaces. The Trk family includes, but is not limited to, TrkA, TrkB, and TrkC. In other embodiments, the neurotrophin receptor is p75^{NTR}, also called p75 or low-affinity nerve growth factor receptor or common neurtotrophin receptor. These receptors may be from any animal species that expresses neurotrophin receptors (e.g. human, murine, rabbit, porcine, equine, etc.), and include full length receptors, their truncated and variant forms, such as those arising by alternate splicing and/or insertion, and naturally-occurring allelic variants, as well as functional derivatives of such receptors.

“Neurotrophin-mediated activity” is a biological activity that is normally modulated (e.g., promoted), either directly or indirectly, in the presence of a neurotrophin. Neurotrophin-mediated activities include, for example, neurotrophin binding to the p75^{NTR} receptor or neurotrophin binding to one of the Trk receptors, the ability to promote neurotrophin receptor dimerization and/or phosphorylation, neuron survival, neuron differentiation including neuron process formation and neurite outgrowth, neurotransmission and biochemical changes such as enzyme induction. A biological activity that is mediated by a particular neurotrophin, e.g. NGF or pro-NGF, is referred to herein by reference to that neurotrophin, e.g. NGF-mediated activity. To determine the ability of a compound to inhibit a neurotrophin-mediated activity, conventional in vitro and in vivo assays can be used. For example, a receptor binding assay, such as the assay described in US 2002/0169182 A1 can be used to assess the extent to which a compound inhibits neurotrophin/receptor binding. Inhibition of neurite survival and outgrowth can be determined using the in vitro assay described by Riopelle et al. in the Can. J. of Phys. and Pharm., 1982, 60: 707.

“Neurotransmission,” as used herein, is a process by which small signaling molecules, termed neurotransmitters, are rapidly passed in a regulated fashion from a neuron to another cell. Typically, following depolarization associated with an incoming action potential, neurotransmitter is secreted from the presynaptic neuronal terminal. The neurotransmitter then diffuses across the synaptic cleft to act on specific receptors on the postsynaptic cell, which is most often a neuron but can also be another cell type (such as muscle fibers at the neuromuscular junction). The action of neurotransmitters can either be excitatory, depolarizing the postsynaptic cell, or inhibitory, resulting in hyperpolarization. Neurotransmission can be rapidly increased or decreased by neuromodulators, which typically act either pre-synaptically or post-synaptically. The neurotrophin family (notably NGF and BDNF) have been shown to have prominent neuromodulatory effects on diverse neuronal types (Lohof et al, Nature. 363(6427):350-3 (1993); Li et al. J Neurosci. 18(24):10231-40. (1998)). BDNF has also been shown to behave like a neurotransmitter, acting directly on target cells to alter their excitability by rapidly and directly gating ion certain channels (Rose et al., Bioessays. 26(11):1185-94. 2004).

There are several simple fashions that neurotransmission can be studied. The release of neurotransmitters from cultured neurons can be directly quantified using HPLC, radiolabled neurotransmitters or other methodologies. Neurotransmission can be estimated by dyes such as FM 1-43, a fluorescent marker of synaptic vesicle cycling. Moreover, neurotransmission between neurons can be directly monitored using standard electrophysiological techniques, as can any direct neurotransmitter-like effects of neurotrophins on ion channel currents. These various methodologies have been used to study the effects of neurotrophins, such as BDNF and NGF, on neurotransmitter release and neurotransmission (Lohof et al.; Li et al.; Rose et al.).

Examples of neurotrophin-mediated activities include, but are not limited to, pain (e.g., inflammatory pain, acute pain, chronic malignant pain, chronic nonmalignant pain, neuropathic pain and migraine), inflammatory disorders and neurological disorders (e.g., neurodegenerative or neuropsychiatric disorders).

The term “pain” as used herein refers to a sensation of discomfort that can range from mild, localized discomfort to agony resulting from the stimulation of neurons (e.g., via neurotrophin activity). Pain is generally associated with tissue damage or inflammation. “Nociception” is the perception of physiological pain and can be grouped generally into four categories including cutaneous pain, somatic pain, visceral pain and neuropathic pain. (It is recognized that certain disorders are associated with more than one category of pain.)

“Cutaneous pain” is caused by injury to the skin or superficial tissues. Cutaneous nociceptors terminate just below the skin, and due to the high concentration of nerve endings, produce a well-defined, localized pain of short duration. Examples of injuries that produce cutaneous pain include, but are not limited to, cuts, burns and lacerations, as well as traumatic injury and post-operative or surgical pain(e.g., at the site of incision).

“Somatic pain” originates from injury, inflammation or disease of the ligaments, tendons, bones, blood vessels, and nerves themselves, and is detected with somatic nociceptors. The scarcity of pain receptors in these areas produces a dull, poorly-localized pain of longer duration than cutaneous pain. Examples of somatic pain include, but are not limited to, sprains, broken bones, arthralgia, vasculitis, myalgia and myofascial pain. Arthralgia refers to pain caused by a joint that has been injured (such as a contusion, break or dislocation) and/or inflamed (e.g., arthritis). Vaculitis refers to inflammation of blood vessels with pain. Myalgia refers to pain originating from the muscles. Myofascial pain refers to pain stemming from injury or inflammation of the fascia and/or muscles. Somatic pain may also be associated with diseases or disorders of the ligaments, tendons, bones, blood vessels and nerves, including, but not limited to, disorders of the musculoskeletal system and connective tissues, and disorders of the circulatory system,

“Visceral” pain is associated with injury, inflammation or disease of the body organs and internal cavities. Disorders that are associated with visceral pain include, but are not limited, to disorders of the circulatory system, respiratory system, gastrointestinal system, genitourinary system, immune system, as well as ear, nose and throat. Visceral pain can also be associated with infectious and parasitic diseases that affect the body organs and tissues. The even greater scarcity of nociceptors in body organs and cavities produces a pain usually more aching and of a longer duration than somatic pain. Visceral pain is extremely difficult to localize, and several injuries to visceral tissue exhibit “referred” pain, where the sensation is localized to an area completely unrelated to the site of injury. For example, myocardial ischaemia (the loss of blood flow to a part of the heart muscle tissue) is possibly the best known example of referred pain; the sensation can occur in the upper chest as a restricted feeling, or as an ache in the left shoulder, arm or even hand. Phantom limb pain is the sensation of pain from a limb that one no longer has or no longer gets physical signals from—an experience almost universally reported by amputees and quadriplegics.

“Neuropathic pain” (“neuralgia”) can occur as a result of injury, inflammation or disease to the nerve tissue itself, for example, caused by a nerve or nerves that are irritated, trapped, pinched, severed or inflamed (neuritis). This can disrupt the ability of the sensory nerves to transmit correct information to the thalamus, and hence the brain interprets painful stimuli even though there is no obvious or documented physiologic cause for the pain. Disorders of the nerve tissue include, but are not limited to, disorders of the nervous system.

Pain can also be categorized as being acute or chronic. “Acute pain” is defined as short-term pain or pain with an easily identifiable cause. It is often fast and sharp and centralized to one area followed by aching pain that can be spread out. “Chronic pain” is defined as constant or intermittent pain that has lasted longer than the expected time of healing, e.g., at least about 2 weeks, 3 weeks, one month, two months, three months, six months or longer.

As used herein the term “inflammatory disease or disorder” includes diseases or disorders which are caused, at least in part, or exacerbated by inflammation, e.g., increased blood flow, edema, activation of immune cells (e.g., proliferation, cytokine production, or enhanced phagocytosis). Inflammatory disorders are generally characterized by heat, redness, swelling, pain and loss of function. The cause of inflammation may be due to physical damage, chemical substances, micro-organisms, tissue necrosis, cancer or other agents. Inflammatory disorders include acute inflammatory disorders, chronic inflammatory disorders, and recurrent inflammatory disorders. Acute inflammatory disorders are generally of relatively short duration, and last for from about a few minutes to about one to two days, although they may last several weeks. The main characteristics of acute inflammatory disorders include increased blood flow, exudation of fluid and plasma proteins (edema) and emigration of leukocytes, such as neutrophils. Chronic inflammatory disorders, generally, are of longer duration, e.g., weeks to months to years or longer, and are associated histologically with the presence of lymphocytes and macrophages and with proliferation of blood vessels and connective tissue. Recurrent inflammatory disorders include disorders which recur after a period of time or which have periodic episodes. Some disorders may fall within one or more categories.

Inflammatory disorders that may be treated according to the methods of the invention include, but are not limited to, inflammation of the nervous system, circulatory system, respiratory system, musculoskeletal and connective tissue system, gastrointestinal system, genitourinary system, eye and adnexa, ear, nose and throat, and endocrine system. Examples of causes of inflammatory disorders include, but are not limited to, microbial infections (e.g., bacterial, viral and fungal infections), physical agents (e.g., burns, radiation, and trauma), chemical agents (e.g., toxins and caustic substances), tissue necrosis and various types of immunologic reactions.

The terms “neurological disorder,” “neurodegenerative disorder” and “neuropsychiatric disorder” can be used interchangeably herein and refer to disorders and states (e.g., a disease state) that are also associated with neurotrophin-mediated biological activity. For example, a neurological disorder can be associated with inappropriate sympathetic or parasympathetic nerve function. A neurological disorder that occurs or has symptoms that occur in the peripheral nervous system is most likely to, but not exclusively, involve the activity of NGF, whereas a neurological disorder that occurs or has symptoms associated with the central nervous system likely involves the activity of NGF and BDNF or a precursor thereof. Examples of neurological states that may be treated according to the methods of the invention include, but are not limited to schizophrenia, biopolar disorder, depression, Alzheimer's disease, epilepsy, cancer, musculoskeletal diseases, multiple sclerosis, amyotrophic lateral sclerosis, stroke, addiction, cerebral ischemia, cardiac disease (e.g., cardiac arrhythmia), neuropathy (e.g., anticancer-agent-intoxicated neuropathy, diabetic neuropathy), retinal pigment degeneration, glaucoma, Huntington's chorea, and Parkinson's disease. As used herein, “neuropathy” is defined as a failure of the nerves that carry information to and from the brain and spinal cord resulting in one or more of pain, loss of sensation, and inability to control muscles. In some cases, the failure of nerves that control blood vessels, intestines, and other organs results in abnormal blood pressure, digestion problems, and loss of other basic body processes. Peripheral neuropathy may involve damage to a single nerve or nerve group (mononeuropathy) or may affect multiple nerves (polyneuropathy).

The term “treated,” “treating” or “treatment” includes the diminishment or alleviation of at least one symptom associated with or caused by the neurotrophin-mediated activity (e.g. pain, inflammatory disorder or a neurological disorder) being treated. In certain embodiments, the treatment comprises the modulation of the interaction of a neurotrophin (e.g., monomer or dimer) and its receptor by a NT/NTR modulating compound, for example a NGF/BDNFINTR, NGF/NTR or BDNF/NTR modulating compound, which would in turn diminish or alleviate at least one symptom associated with or caused by the neurotrophin-mediated activity being treated. For example, treatment can be diminishment of one or several symptoms of a disorder or complete eradication of a disorder.

As used herein, the phrase “therapeutically effective amount” of the compound is the amount necessary or sufficient to treat or prevent a neurotrophin-mediated activity, e.g., pain, inflammatory disorder or a neurological disorder, e.g., to prevent the various morphological and somatic symptoms of a neurotrophin-mediated activity. In an example, an effective amount of the compound is the amount sufficient to alleviate at least one symptom of the disorder, e.g., pain, in a subject.

The term “subject” is intended to include animals, which are capable of suffering from or afflicted with a neurotrophin-associated state or neurotrophin-associated disorder, or any disorder involving, directly or indirectly, neurotrophin signaling. Examples of subjects include mammals, e.g., humans, dogs, cows, horses, pigs, sheep, goats, cats, mice, rabbits, rats, and transgenic non-human animals. In certain embodiments, the subject is a human, e.g., a human suffering from, at risk of suffering from, or potentially capable of suffering from a neurotrophin-associated state or neurotrophin disorder.

The language “NT/NTR modulator” refers to compounds that modulate, i.e., inhibit, promote or otherwise alter the interaction of a neurotrophin with a neurotrophin receptor. For example, “NGF/NTR modulator” refers to compounds that modulate, e.g., inhibit, promote, or otherwise alter, the interaction of NGF (or proNGF) with p75^{NTR}, TrkA, or p75^{NTR} and TrkA. The language “BDNF/NTR modulator” refers to compounds that modulate, e.g., inhibit, promote, or otherwise alter, the interaction of BDNF (or proBDNF) with p75^{NTR}, TrkB or p75^{NTR} and TrkB. Likewise, the language “NGF/BDNF/NTR modulator” refers to compounds that modulate, e.g., inhibit, promote, or otherwise alter, the interaction of NGF (or proNGF) with p75^{NTR}, TrkA or p75^{NTR} and TrkA, and the interaction of TrkA, BDNF (proBDNF) with p75^{NTR}, TrkB or p75^{NTR} and TrkB. Examples of NT/NTR modulators (e.g., NGF/NTR modulators, BDNF/NTR modulators, and NGF/BDNF/NTR modulators) include compounds of Formula 1, Formula 2, Formula 3, Formula 3A, Formula 4, Formula 5 and Formula 6, including salts thereof, e.g., a pharmaceutically acceptable salt. Additional examples of NT/NTR modulators (e.g., NGF/NTR modulators, BDNF/NTR modulators, and NGF/BDNF/NTR modulators) include compounds of Table 1, Table 2, Table 3, and Table 4 or derivatives and fragments thereof, including salts thereof, e.g., a pharmaceutically acceptable salt.

In a preferred embodiment of the invention, exemplary BDNF/NTR modulators are shown in Table 1, exemplary NGF/BDNF/NTR modulators are shown in Table 2, and exemplary NGF/NTR modulators are shown in Table 3.

In another preferred emdodiment, the NT/NTR modulators of the invention are selected from the group consisting of 5-[5-(2-Bromo-4-nitrophenyl)furan-2-ylmethylene]-2-thioxo-dihydro-pyrimidine-4,6-dione, 5-[5-(2-Methyl-5-nitrophenyl)furan-2-ylmethylene]-2-thioxo-thiazolidin-4-one, 5-[5-(2,5-Dimethyl-3-nitrophenyl)furan-2-ylmethylene]-2-thioxo-dihydro-pyrimidine-4,6-dione, 5-[5-(2-Chloro-4-nitrophenyl)furan-2-ylmethylene]-2-thioxo-dihydro-pyrimidine-4,6-dione, 5-[5-(2-Chloro-4-nitrophenyl)furan-2-ylmethylene]-2-thioxo-thiazolidin-4-one, 4-[5-(4,6-Dioxo-2-thioxo-tetrahydro-pyrimidin-5-ylidenemethyl)furan-2-yl]benzoic acid, 5-[5-(2-Methyl-4-nitrophenyl)furan-2-ylmethylene]-2-thioxo-thiazolidin-4-one, 5-[5-(4-Methoxy-3-nitrophenyl)furan-2-ylmethylene]-2-thioxo-dihydro-pyrimidine-4,6-dione, 4-[5-(3,5-Dioxo-1-m-tolyl-pyrazolidin-4-ylidenemethyl)furan-2-yl]benzenesulfonamide, 3-{5-[5-(4-Nitrophenyl)furan-2-ylmethylene]-4-oxo-2-thioxo-thiazolidin-3-yl}-propionic acid, 5-[5-(2-Chloro-4-nitrophenyl)furan-2-ylmethylene]-pyrimidine-2,4,6-trione, 4-[5-(3-Carboxymethyl-4-oxo-2-thioxo-thiazolidin-5-ylidenemethyl)furan-2-yl]benzoic acid butyl ester, 4-Chloro-3-{5-[1-(4-fluorobenzyl)-2,5-dioxo-imidazolidin-4-ylidenemethyl]furan-2-yl}benzoic acid, {5-[5-(4-Methyl-3-nitrophenyl)furan-2-ylmethylene]-4-oxo-2-thioxo-thiazolidin-3-yl}-acetic acid, {5-[5-(3-Methyl-4-nitrophenyl)furan-2-ylmethylene]-4-oxo-2-thioxo-thiazolidin-3-yl}-acetic acid, 3-[5-(2,5-Dioxo-1-phenyl-imidazolidin-4-ylidenemethyl)furan-2-yl]-4-methylbenzoic acid, 4-[5-(1-Methyl-2,4,6-trioxo-tetrahydro-pyrimidin-5-ylidenemethyl)furan-2-yl]benzoic acid, 5-[5-(2,5-Dimethyl-4-nitrophenyl)furan-2-ylmethylene]-pyrimidine-2,4,6-trione, 4-[5-(4,6-Dioxo-2-thioxo-tetrahydro-pyrimidin-5-ylidenemethyl)furan-2-yl]benzoic acid methyl ester, 5-[5-(4-Methoxy-3-nitrophenyl)furan-2-ylmethylene]-2-thioxo-thiazolidin-4-one, 3-[5-(3-Ethoxycarbonylmethyl-2,4-dioxo-thiazolidin-5-ylidenemethyl)furan-2-yl]-4-methylbenzoic acid, 3-[5-(2,5-Dioxo-imidazolidin-4-ylidenemethyl)furan-2-yl]-4-methylbenzoic acid, 4-[5-(2-Chloro-4-nitrophenyl)furan-2-ylmethylene]-1-(3,4-dimethylphenyl)-pyrazolidine-3,5-dione, 2-Chloro-4-[5-(3-ethoxycarbonylmethyl-2,4-dioxo-thiazolidin-5-ylidenemethyl)furan-2-yl]benzoic acid, 5-[5-(3,4-Dimethyl-5-nitrophenyl)furan-2-ylmethylene]-2-thioxo-dihydro-pyrimidine-4,6-dione, 5-[5-(4-Nitrophenyl)furan-2-ylmethylene]-pyrimidine-2,4,6-trione, 2-Chloro-4-{5-[1-(2-fluorobenzyl)-2,5-dioxo-imidazolidin-4-ylidenemethyl]furan-2-yl}benzoic acid, 2-Chloro-5-{5-[1-(4-fluorobenzyl)-2,5-dioxo-imidazolidin-4-ylidenemethyl]furan-2-yl}benzoic acid, 3-{5-[3-(1-Methoxycarbonyl-ethyl)-2,4-dioxo-thiazolidin-5-ylidenemethyl]furan-2-yl}-4-methylbenzoic acid, 2-Chloro-4-{5-[3-(1-methoxycarbonyl-ethyl)-2,4-dioxo-thiazolidin-5-ylidenemethyl]furan-2-yl}benzoic acid, 5-[5-(4-Methoxy-3-nitrophenyl)furan-2-ylmethylene]-1,3-dimethyl-pyrimidine-2,4,6-trione, 4-[5-(1-benzyl-2,4,6-trioxo-tetrahydro-pyrimidin-5-ylidenemethyl)furan-2-yl]benzoic acid methyl ester, 5-[5-(2-Chloro-4-nitrophenyl)furan-2-ylmethylene]-thiazolidine-2,4-dione, 3-(2-Methoxy-ethyl)-5-[5-(3-nitrophenyl)furan-2-ylmethylene]-2-thioxo-thiazolidin-4-one, 4-[5-(3-Methoxycarbonylmethyl-2,4-dioxo-thiazolidin-5-ylidenemethyl)furan-2-yl]-3-methylbenzoic acid, 2-Chloro-4-{5-[1-(3-chlorophenyl)-2,5-dioxo-imidazolidin-4-ylidenemethyl]furan-2-yl}benzoic acid, 4-{5-[3-(4-Methoxybenzyl)-4-oxo-2-thioxo-thiazolidin-5-ylidenemethyl]furan-2-yl}benzenesulfonamide, {5-[5-(4-Nitrophenyl)furan-2-ylmethylene]-4-oxo-2-thioxo-thiazolidin-3-yl}-acetic acid, 1-Methyl-5-[5-(4-nitrophenyl)furan-2-ylmethylene]-pyrimidine-2,4,6-trione, {5-[5-(2-Methyl-4-nitrophenyl)furan-2-ylmethylene]-4-oxo-2-thioxo-thiazolidin-3-yl}-acetic acid, 4-[5-(2,5-Dioxo-imidazolidin-4-ylidenemethyl)furan-2-yl]-3-methylbenzoic acid, 2-Chloro-5-[5-(2,4,6-trioxo-tetrahydro-pyrimidin-5-ylidenemethyl)furan-2-yl]benzoic acid butyl ester, 2-Chloro-5-{5-[1-(3-chlorophenyl)-2,5-dioxo-imidazolidin-4-ylidenemethyl]furan-2-yl}benzoic acid, 4-[5-(4-Chloro-3-nitrophenyl)furan-2-ylmethylene]-1-m-tolyl-pyrazolidine-3,5-dione, 4-[5-(4-Chloro-3-nitrophenyl)furan-2-ylmethylene]-1-m-tolyl-pyrazolidine-3,5-dione, 4-[5-(3-Methyl-4-oxo-2-thioxo-thiazolidin-5-ylidenemethyl)furan-2-yl]benzenesulfonamide, 4-{5-[1-(4-Fluorobenzyl)-2,5-dioxo-imidazolidin-4-ylidenemethyl]furan-2-yl}-3-methylbenzoic acid methyl ester, 5-[5-(2-Chloro-4-nitrophenyl)furan-2-ylmethylene]-2-thioxo-imidazolidin-4-one, 3-[5-(2,5-Dioxo-1-imidazolidin-4-ylidenemethyl)furan-2-yl]-2-methylbenzoic acid, 4-{5-[1-(4-Fluorophenyl)-3-methyl-5-oxo-1,5-dihydro-pyrazol-4-ylidenemethyl]furan-2-yl}benzoic acid, {5-[5-(4-Nitrophenyl)furan-2-ylmethylene]-2,4-dioxo-thiazolidin-3-yl}-acetic acid methyl ester, 5-[5-(4-Chloro-3-nitrophenyl)furan-2-ylmethylene]-2-thioxo-thiazolidin-4-one, 2-{5-[5-(4-Nitrophenyl)furan-2-ylmethylene]-4-oxo-2-thioxo-thiazolidin-3-yl}-pentanoic acid, 1-Methyl-5-[5-(2-methyl-4-nitrophenyl)furan-2-ylmethylene]-pyrimidine-2,4,6-trione, 4-[5-(2,5-Dioxo-1-phenyl-imidazolidin-4-ylidenemethyl)furan-2-yl]benzoic acid ethyl ester, 3-Ethyl-5-[5-(4-nitrophenyl)furan-2-ylmethylene]-2-thioxo-imidazolidin-4-one, 4-[5-(2-Methoxy-4-nitrophenyl)furan-2-ylmethylene]-3-phenyl-4H-isoxazol-5-one, 3-Ethyl-5-[5-(2-methoxy-4-nitrophenyl)furan-2-ylmethylene]-thiazolidine-2,4-dione, 5-[5-(4-Nitrophenyl)furan-2-ylmethylene]-3-pyridin-3-ylmethyl-2-thioxo-thiazolidin-4-one, 5-[5-(2-Methoxy-4-nitrophenyl)furan-2-ylmethylene]-3-methyl-2-thioxo-thiazolidin-4-one, {5-[5-(2-Methyl-4-nitrophenyl)furan-2-ylmethylene]-2,4-dioxo-thiazolidin-3-yl}-acetic acid ethyl ester, 4-[5-(2-Chloro-4-nitrophenyl)furan-2-ylmethylene]-2-propylsulfanyl-4H-thiazol-5-one, 4-[5-(3,5-Dioxo-1-m-tolyl-pyrazolidin-4-ylidenemethyl)furan-2-yl]benzoic acid ethyl ester, 2-{5-[5-(4-Methyl-3-nitrophenyl)furan-2-ylmethylene]-2,4-dioxo-thiazolidin-3-yl}-propionic acid methyl ester, 2-Chloro-4-[5-(4,6-dioxo-2-thioxo-tetrahydro-pyrimidin-5-ylidenemethyl)furan-2-yl]benzoic acid, 4-{5-[1-(2-Fluorophenyl)-2,4,6-trioxo-tetrahydro-pyrimidin-5-ylidenemethyl]furan-2-yl}-3-methylbenzoic acid, 2-Chloro-4-[5-(3-methoxycarbonylmethyl-2,4-dioxo-thiazolidin-5-ylidenemethyl)furan-2-yl]benzoic acid, 4-[5-(3-Carboxymethyl-4-oxo-2-thioxo-thiazolidin-5-ylidenemethyl)furan-2-yl]benzoic acid ethyl ester, 3-[5-(3-Carboxymethyl-4-oxo-2-thioxo-thiazolidin-5-ylidenemethyl)furan-2-yl]benzoic acid methyl ester, 1-(4-Fluorophenyl)-5-[5-(3-nitrophenyl)furan-2-ylmethylene]-2-thioxo-dihydro-pyrimidine-4,6-dione, 5-[5-(2,3-Dimethyl-4-nitrophenyl)furan-2-ylmethylene]-2-thioxo-dihydro-pyrimidine-4,6-dione, 4-Chloro-3-{5-[1-(2-fluorobenzyl)-2,5-dioxo-imidazolidin-4-ylidenemethyl]furan-2-yl}benzoic acid, 4-[5-(3-Ethyl-2,4-dioxo-thiazolidin-5-ylidenemethyl)furan-2-yl]benzoic acid, {4-Oxo-5-[5-(3-sulfamoylphenyl)furan-2-ylmethylene]-2-thioxo-thiazolidin-3-yl}-acetic acid, 2-Chloro-5-[5-(2,4,6-trioxo-tetrahydro-pyrimidin-5-ylidenemethyl)furan-2-yl]benzoic acid, 3-Methyl-4-[5-(3-methyl-2,4-dioxo-thiazolidin-5-ylidenemethyl)furan-2-yl]benzoic acid, 3-[5-(1-Methyl-2,4,6-trioxo-tetrahydro-pyrimidin-5-ylidenemethyl)furan-2-yl]benzoic acid, 1-Methyl-5-[5-(3-nitrophenyl)furan-2-ylmethylene]-pyrimidine-2,4,6-trione, 4-[5-(3-Methoxycarbonylmethyl-2,4-dioxo-thiazolidin-5-ylidenemethyl)furan-2-yl]benzoic acid, 2-Chloro-4-[5-(3-methyl-2,4-dioxo-thiazolidin-5-ylidenemethyl)furan-2-yl]benzoic acid, 5-[5-(2,3-Dimethyl-4-nitrophenyl)furan-2-ylmethylene]-pyrimidine-2,4,6-trione, {5-[5-(4-Chloro-3-nitrophenyl)furan-2-ylmethylene]-4-oxo-2-thioxo-thiazolidin-3-yl}-acetic acid, 4-[5-(3,5-Dioxo-1-m-tolyl-pyrazolidin-4-ylidenemethyl)furan-2-yl]benzoic acid, 4-{5-[5-(3-Nitrophenyl)furan-2-ylmethylene]-4-oxo-2-thioxo-thiazolidin-3-yl}-butyric acid, 2-Chloro-5-[5-(5-oxo-2-thioxo-imidazolidin-4-ylidenemethyl)furan-2-yl]benzoic acid, 4-[5-(2,4,6-Trioxo-tetrahydro-pyrimidin-5-ylidenemethyl)furan-2-yl]benzoic acid, 5-[5-(3-Nitrophenyl)furan-2-ylmethylene]-thiazolidine-2,4-dione, 5-[5-(2-Methyl-5-nitrophenyl)furan-2-ylmethylene]-thiazolidine-2,4-dione, 5-[5-(3-Nitrophenyl)furan-2-ylmethylene]-2-thioxo-imidazolidin-4-one, {5-[5-(4-Chloro-3-nitrophenyl)furan-2-ylmethylene]-4-oxo-2-thioxo-thiazolidin-3-yl }-acetic acid, 4-[5-(3,5-Dioxo-1-m-tolyl-pyrazolidin-4-ylidenemethyl)furan-2-yl]benzoic acid, 4-{5-[5-(3-Nitrophenyl)furan-2-ylmethylene]-4-oxo-2-thioxo-thiazolidin-3-yl}-butyric acid, 2-Chloro-5-[5-(5-oxo-2-thioxo-imidazolidin-4-ylidenemethyl)furan-2-yl]benzoic acid, 4-[5-(2,4,6-Trioxo-tetrahydro-pyrimidin-5-ylidenemethyl)furan-2-yl]benzoic acid, 5-[5-(3-Nitrophenyl)furan-2-ylmethylene]-thiazolidine-2,4-dione, 5-[5-(2-Methyl-5-nitrophenyl)furan-2-ylmethylene]-thiazolidine-2,4-dione, 5-[5-(3-Nitrophenyl)furan-2-ylmethylene]-2-thioxo-imidazolidin-4-one, 2-Chloro-5-[5-(5-oxo-2-thioxo-imidazolidin-4-ylidenemethyl)furan-2-yl]benzoic acid methyl ester, 5-[5-(2-Methyl-4-nitrophenyl)furan-2-ylmethylene]-thiazolidine-2,4-dione, 5-[5-(4-Chloro-3-nitrophenyl)furan-2-ylmethylene]-2-thioxo-imidazolidin-4-one, 5-[5-(2-Methyl-4-nitrophenyl)furan-2-ylmethylene]-2-thioxo-imidazolidin-4-one, 5-[5-(3-Methyl-4-nitrophenyl)furan-2-ylmethylene]-2-thioxo-imidazolidin-4-one, 5-[5-(3-Methyl-4-nitrophenyl)furan-2-ylmethylene]-pyrimidine-2,4,6-trione, 5-[5-(4-Chloro-3-nitrophenyl)furan-2-ylmethylene]-thiazolidine-2,4-dione, 4-{5-[5-(4-Nitrophenyl)furan-2-ylmethylene]-4-oxo-2-thioxo-thiazolidin-3-yl}-butyric acid, 4-[5-(4,6-Dioxo-2-thioxo-tetrahydro-pyrimidin-5-ylidenemethyl)furan-2-yl]benzoic acid ethyl ester, 5-[5-(2-Bromo-4-nitrophenyl)furan-2-ylmethylene]-2-thioxo-thiazolidin-4-one, 5-[5-(2-Methoxy-4-nitrophenyl)furan-2-ylmethylene]-2-thioxo-thiazolidin-4-one, 4-[5-(3,5-Dioxo-1-p-tolyl-pyrazolidin-4-ylidenemethyl)furan-2-yl]benzenesulfonamide, 5-[5-(2-Bromo-4-nitrophenyl)furan-2-ylmethylene]-2-imino-thiazolidin-4-one, 4-{5-[1-(4-Ethylphenyl)-3,5-dioxo-pyrazolidin-4-ylidenemethyl]furan-2-yl}benzenesulfonamide, 4-[5-(4,6-Dioxo-2-thioxo-tetrahydro-pyrimidin-5-ylidenemethyl)furan-2-yl]benzenesulfonamide, 5-[5-(4-Methyl-3-nitrophenyl)furan-2-ylmethylene]-2-thioxo-dihydro-pyrimidine-4,6-dione, 3-[5-(4,6-Dioxo-2-thioxo-tetrahydro-pyrimidin-5-ylidenemethyl)furan-2-yl]benzoic acid methyl ester, 2-Chloro-4-[5-(5-oxo-2-thioxo-imidazolidin-4-ylidenemethyl)furan-2-yl]benzoic acid, 5-[5-(2-Methyl-3-nitrophenyl)furan-2-ylmethylene]-pyrimidine-2,4,6-trione, 3-Ethyl-5-[5-(4-nitrophenyl)furan-2-ylmethylene]-thiazolidine-2,4-dione, 2-Methylsulfanyl-4-[5-(4-nitrophenyl)furan-2-ylmethylene]-4H-thiazol-5-one, 4-[5-(4,6-Dioxo-2-thioxo-tetrahydro-pyrimidin-5-ylidenemethyl)furan-2-yl]benzenesulfonamide, 5-[5-(4-Methyl-3-nitrophenyl)furan-2-ylmethylene]-2-thioxo-dihydro-pyrimidine-4,6-dione, 3-[5-(4,6-Dioxo-2-thioxo-tetrahydro-pyrimidin-5-ylidenemethyl)furan-2-yl]benzoic acid methyl ester, 2-Chloro-4-[5-(5-oxo-2-thioxo-imidazolidin-4-ylidenemethyl)furan-2-yl]benzoic acid, 5-[5-(2-Methyl-3-nitrophenyl)furan-2-ylmethylene]-pyrimidine-2,4,6-trione, 3-Ethyl-5-[5-(4-nitrophenyl)furan-2-ylmethylene]-thiazolidine-2,4-dione, 2-Methylsulfanyl-4-[5-(4-nitrophenyl)furan-2-ylmethylene]-4H-thiazol-5-one, 2-Methylsulfanyl-4-[5-(3-nitrophenyl)furan-2-ylmethylene]-4H-thiazol-5-one, {5-[5-(4-Chloro-3-nitrophenyl)furan-2-ylmethylene]-2,4-dioxo-thiazolidin-3-yl}-acetic acid ethyl ester, 5-[5-(4-Methyl-3-nitrophenyl)furan-2-ylmethylene]-imidazolidine-2,4-dione, 2-Chloro-5-[5-(3-ethoxycarbonylmethyl-2,4-dioxo-thiazolidin-5-ylidenemethyl)furan-2-yl]benzoic acid, {5-[5-(3-Nitrophenyl)furan-2-ylmethylene]-2,4-dioxo-thiazolidin-3-yl}-acetic acid ethyl ester, {5-[5-(4-Nitrophenyl)furan-2-ylmethylene]-2,4-dioxo-thiazolidin-3-yl}-acetic acid ethyl ester, {5-[5-(2-Methoxy-4-nitrophenyl)furan-2-ylmethylene]-2,4-dioxo-thiazolidin-3-yl}-acetic acid ethyl ester, 1-(4-Fluorophenyl)-4-[5-(4-nitrophenyl)furan-2-ylmethylene]-pyrazolidine-3,5-dione, 3-(2-Methoxy-ethyl)-5-[5-(4-nitrophenyl)furan-2-ylmethylene]-2-thioxo-thiazolidin-4-one, 4-[5-(3-Ethoxycarbonylmethyl-2,4-dioxo-thiazolidin-5-ylidenemethyl)furan-2-yl]-3-methylbenzoic acid, 3-[5-(1-benzyl-2,4,6-trioxo-tetrahydro-pyrimidin-5-ylidenemethyl)furan-2-yl]benzoic acid, 5-[5-(2-Methoxy-5-nitrophenyl)furan-2-ylmethylene]-3-phenyl-imidazolidine-2,4-dione, 4-{5-[1-(3,5-Dimethylphenyl)-4,6-dioxo-2-thioxo-tetrahydro-pyrimidin-5-ylidenemethyl]furan-2-yl}benzoic acid, {5-[5-(2-Methoxy-4-nitrophenyl)furan-2-ylmethylene]-2,4-dioxo-thiazolidin-3-yl}-acetic acid methyl ester, 4-{5-[1-(4-Fluorobenzyl)-2,5-dioxo-imidazolidin-4-ylidenemethyl]furan-2-yl}benzoic acid, 5-[5-(4-Chloro-3-nitrophenyl)furan-2-ylmethylene]-3-phenyl-imidazolidine-2,4-dione, 4-{5-[1-(3-Chlorophenyl)-2,5-dioxo-imidazolidin-4-ylidenemethyl]furan-2-yl}benzoic acid methyl ester, 3-(2-Fluorobenzyl)-5-[5-(4-methyl-3-nitrophenyl)furan-2-ylmethylene]-imidazolidine-2,4-dione, 3-{5-[1-(3-Chlorophenyl)-2,5-dioxo-imidazolidin-4-ylidenemethyl]furan-2-yl}-2-methylbenzoic acid, 3-(2-Fluorobenzyl)-5-[5-(2-methyl-4-nitrophenyl)furan-2-ylmethylene]-imidazolidine-2,4-dione, 5-[5-(2-Methoxy-5-nitrophenyl)furan-2-ylmethylene]-3-phenyl-imidazolidine-2,4-dione, 4-{5-[1-(3,5-Dimethylphenyl)-4,6-dioxo-2-thioxo-tetrahydro-pyrimidin-5-ylidenemethyl]furan-2-yl}benzoic acid, {5-[5-(2-Methoxy-4-nitrophenyl)furan-2-ylmethylene]-2,4-dioxo-thiazolidin-3-yl}-acetic acid methyl ester, 4-{5-[1-(4-Fluorobenzyl)-2,5-dioxo-imidazolidin-4-ylidenemethyl]furan-2-yl}benzoic acid, 5-[5-(4-Chloro-3-nitrophenyl)furan-2-ylmethylene]-3-phenyl-imidazolidine-2,4-dione, 4-{5-[1-(3-Chlorophenyl)-2,5-dioxo-imidazolidin-4-ylidenemethyl]furan-2-yl}benzoic acid methyl ester, 3-(2-Fluorobenzyl)-5-[5-(4-methyl-3-nitrophenyl)furan-2-ylmethylene]-imidazolidine-2,4-dione, 3-{5-[1-(3-Chlorophenyl)-2,5-dioxo-imidazolidin-4-ylidenemethyl]furan-2-yl}-2-methylbenzoic acid, 3-(2-Fluorobenzyl)-5-[5-(2-methyl-4-nitrophenyl)furan-2-ylmethylene]-imidazolidine-2,4-dione, {5-[5-(2-Chloro-4-nitrophenyl)furan-2-ylmethylene]-2,4-dioxo-thiazolidin-3-yl}-acetic acid ethyl ester, 3-(2-Fluorobenzyl)-5-[5-(2-methoxy-4-nitrophenyl)furan-2-ylmethylene]-imidazolidine-2,4-dione, 2-Chloro-5-[5-(3,5-dioxo-1-m-tolyl-pyrazolidin-4-ylidenemethyl)furan-2-yl]benzoic acid, 3-{4-Oxo-5-[5-(4-sulfamoylphenyl)furan-2-ylmethylene]-2-thioxo-thiazolidin-3-yl-propionic acid and 4-{4-[5-(4-Methoxy-2-nitro-phenyl)-furan-2-ylmethylene]-3-methyl-5-oxo-4,5-dihydro-pyrazol-1-yl}-benzoic acid.

Modulators of Neurotrophin/Neurotrophin Receptor Interaction

In one aspect, the present invention provides compounds which modulate the interaction of a neurotrophin with a neurotrophin receptor. In certain embodiments, the compounds modulate the interaction of nerve growth factor (NGF) and/or brain-derived neurotrophic factor (BDNF) and/or a precursor thereof with a neurotrophin receptor. In other embodiments the compound modulates the interaction of NGF and/or BDNF and/or a precursor thereof with the p75^{NTR} receptor. In still other embodiments, the compound also modulates the interaction of NGF (or proNGF) with the TrkA receptor. In other embodiments the compound also modulates the interaction of BDNF (or proBDNF) with the TrkB receptor. In further embodiments, the compound modulates the interaction of NGF with both the p75^{NTR} and TrkA receptor, and also modulates the interaction of BDNF with both the p75^{NTR} and TrkB receptor.

The compounds of the invention have at least two of the following structural attributes: (1) a first electronegative atom or functional group; (2) a second electronegative atom or functional group; (3) a third electronegative atom or functional group; (4) a fourth electronegative atom or functional group; and (5) a hydrophobic moiety. A compound having two or more of these structural attributes is referred to herein as an NT/NTR, “NGF/NTR”, “BDNF/NTR” or “NGF/BDNF/NTR modulator,” depending on the specific biological activity of the particular compound. Preferably, the NT/NTR modulator has at least three of the foregoing attributes, more preferably at least four such attributes. Most preferably, the NT/NTR modulator has each of the five foregoing attributes.

An electronegative atom of the NT/NTR modulator bears a full or partial negative charge under physiological conditions and can, therefore, interact electrostatically with the positively charged side chain of an NGF lysine residue. This will be an interaction, such as, for example, a hydrogen bond, an ion/ion interaction, an ion/dipole interaction or a dipole/dipole interaction. The hydrophobic region or moiety of the NT/NTR modulator can interact with a hydrophobic region of NGF via a hydrophobic interaction. Without being bound by theory, it is believed that compounds having the disclosed structural features can interact with NGF in such a way as to interfere with, and thereby modulate, the interaction of NGF and p75^{NTR}.

Preferred NT/NTR modulators of the invention comprise a molecular scaffold or framework, to which the electronegative atoms or functional groups are attached, either directly or via an intervening moiety. The scaffold can be, for example, a cyclic or polycyclic moiety, such as a monocyclic, bicyclic or tricyclic moiety, and can include one or more hydrocarbyl or heterocyclic rings. Preferably, the scaffold includes three or more bonded, five- or six-membered rings. The molecular scaffold presents the attached electronegative atoms, electronegative functional groups or a combination thereof, in the proper configuration or orientation for interaction with the appropriate residues of NGF and BDNF. In addition, the molecular scaffold, such as polycyclic system, or a portion thereof, can serve as the hydrophobic group which interacts with hydrophobic residues of NGF and BDNF, as described above.

In one embodiment, the NT/NTR modulator is of general Formula 1, 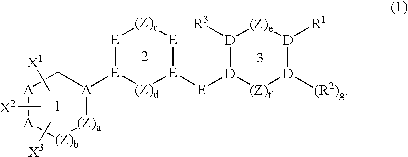
In Formula 1, A, E and D are each, independently, an sp²- or sp³-hybridized carbon, nitrogen, oxygen or sulfur atom; X¹, X² and X³ are each, independently, a hydrogen atom, C₁-C₆-alkyl, an electronegative atom, such as oxygen, sulfur, fluorine, chlorine, bromine or iodine, or an electronegative functional group, such as aryl, alkylcarbonyl, alkylthiocarbonyl, alkoxycarbonyl, aminocarbonyl, —OH, —CN, —CO₂H, —CO₂C₁-C₆, —[(CH₂)₁₋₆]CO₂H, aryl-CO₂H, aryl-CO₂C₁-C₆, —O[(CH₂)₀₋₆]CH₃, —SO₃H, —SO₂H, —SO₂NH₂, —PO₃H₂, —NO₂, —ONO₂, —CNO, —SH, —CNS, —OSO₃H, —OC(O)(OH), —O—, —S—, halomethyl, dihalomethyl, trihalomethyl or N(R⁴)R⁵, where R⁴ and R⁵ are each, independently, H, aryl, C₁-C₆-alkyl; esters thereof, salts thereof, and any combination thereof; R¹, R² and R³ are each, independently, a hydrogen atom, C₁-C₆-alkyl, an electronegative atom, such as oxygen, sulfur, fluorine, chlorine, bromine or iodine, or an electronegative functional group, such as aryl, aryl-CO₂H, aryl-CO₂C₁-C₆, alkylcarbonyl, alkylthiocarbonyl, alkoxycarbonyl, aminocarbonyl, —OH, —CN, —CO₂H, —CO₂C₁-C₆, —[(CH₂)₁₋₆]CO₂H, —O[(CH₂)₀₋₆]CH₃, —SO₃H, —SO₂H, —SO₂NH₂, —PO₃H₂, —NO₂, —ONO₂, —CNO, —SH, —CNS, —OSO₃H, —OC(O)(OH), —O—, —S—, halomethyl, dihalomethyl, trihalomethyl or N(R⁴)R⁵, where R⁴ and R⁵ are each, independently, H, aryl, C₁-C₆-alkyl; esters thereof, salts thereof, and any combination thereof; Z is independently selected from the group consisting of C═S, C, O, S, CH, C(O), N, NH, C-alkyl, N-alkyl, C-aryl, N-aryl, N-cycloalkyl and N—P, where P is a carbohydrate moiety, such as a monosaccharide group, for example, a fucosyl, glucosyl, galactosyl, mannosyl, fructosyl, gulosyl, idosyl, talosyl, allosyl, altrosyl, ribosyl, arabinosyl, xylosyl or lyxosyl group; a, b, c, d, e, f and g are each 0 or 1, provided that at least one of a and b is 1, at least one of c and d is 1, and at least one of e and f is 1.

In one embodiment of the compounds of Formula 1, R¹, R² and R³ are selected from the group consisting of substituted phenylene, naphthylene, quinolylene and other substituted aromatic and heteroaromatic groups. R¹, R² and R³ can also be a substituted ethylnyl or poly(ethylnyl) group. Suitable identities for R¹, R² and R³ include, but are not limited to, the groups shown below:

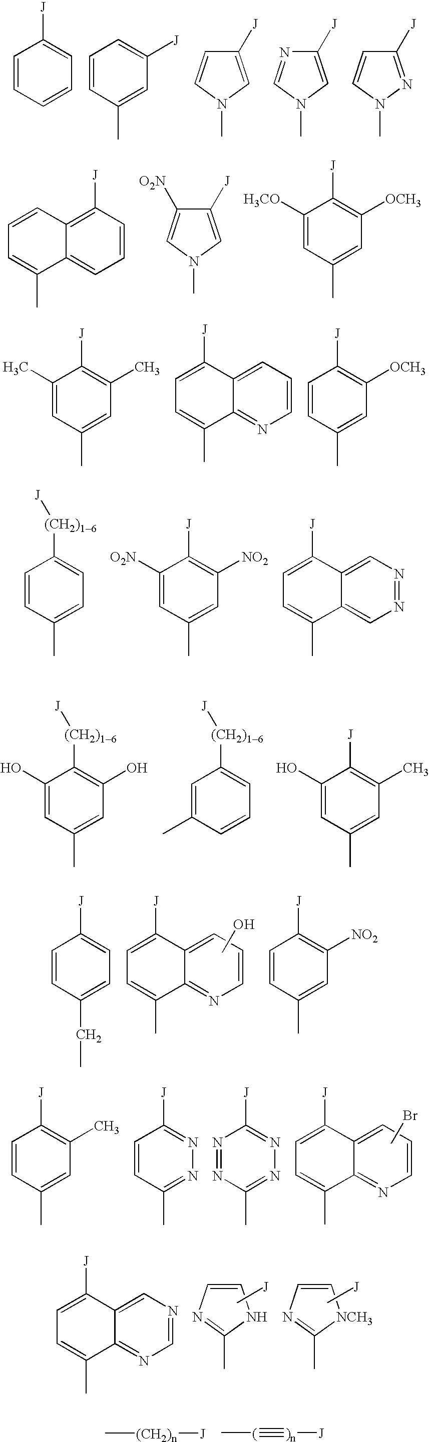

In each of these groups, J can be any of the electronegative atoms or groups described in the definition of R¹ in Formula 1. Preferably, J is selected from the group consisting of —OH, —CN, —NO₂, —CO₂H, —CO₂C₁-C₆, —(CH₂)₁₋₆COOH, —SO₃H, —SO₂H, —F, —Cl, —Br, —I, —PO₃H₂, —CF₃, —SO₂N(CH₃)₂, —C(O)NH₂, —C(O)CH₃, —C(O)OCH₃, —C(O)CN, —CH₂F, —CH₂Cl, —CF₂H, —CCl₂H, and —CCl₃.

A preferred compound of Formula I is represented below: 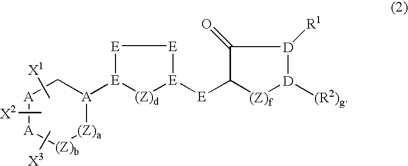
In Formula 2, A, E and D are each, independently, an sp²- or sp³-hybridized carbon, nitrogen, oxygen or sulfur atom; X¹, X² and X³ are each, independently, a hydrogen atom, C₁-C₆-alkyl, an electronegative atom, such as oxygen, sulfur, fluorine, chlorine, bromine or iodine, or an electronegative functional group, such as aryl, alkylcarbonyl, alkylthiocarbonyl, alkoxycarbonyl, aminocarbonyl, —OH, —CN, —CO₂H, —CO₂C₁-C₆, —[(CH₂)₁₋₆]CO₂H, aryl-CO₂H, aryl-CO₂C₁-C₆, —O[(CH₂)₀₋₆]CH₃, —SO₃H, —SO₂H, —SO₂NH₂, —PO₃H₂, —NO₂, —ONO₂, —CNO, —SH, —CNS, —OSO₃H, —OC(O)(OH), —O—, —S—, halomethyl, dihalomethyl, trihalomethyl or N(R⁴)R⁵, where R⁴ and R⁵ are each, independently, H, aryl, C₁-C₆-alkyl; esters thereof, salts thereof, and any combination thereof; R¹ and R² are each, independently, a hydrogen atom, C₁-C₆-alkyl, an electronegative atom, such as oxygen, sulfur, fluorine, chlorine, bromine or iodine, or an electronegative functional group, such as aryl, aryl-CO₂H, aryl-CO₂C₁-C₆, alkylcarbonyl, alkylthiocarbonyl, alkoxycarbonyl, aminocarbonyl, —OH, —CN, —CO₂H, —CO₂C₁-C₆, —[(CH₂)₁₋₆]CO₂H, —O[(CH₂)₀₋₆]CH₃, —SO₃H, —SO₂H, —SO₂NH₂, —PO₃H₂, —NO₂, —ONO₂, —CNO, —SH, —CNS, —OSO₃H, —OC(O)(OH), —O—, —S—, halomethyl, dihalomethyl, trihalomethyl or N(R⁴)R⁵, where R⁴ and R⁵ are each, independently, H, aryl, C₁-C₆-alkyl; esters thereof, salts thereof, and any combination thereof; Z is independently selected from the group consisting of C═S, C, O, S, CH, C(O), N, NH, C-alkyl, N-alkyl, C-aryl, N-aryl, N-cycloalkyl and N—P, where P is a carbohydrate moiety, such as a monosaccharide group, for example, a fucosyl, glucosyl, galactosyl, mannosyl, fructosyl, gulosyl, idosyl, talosyl, allosyl, altrosyl, ribosyl, arabinosyl, xylosyl or lyxosyl group; a, b and g are each 0 or 1, provided that at least one of a and b is 1; d and f are each 1.

Another preferred compound of Formula 1 is represented below: 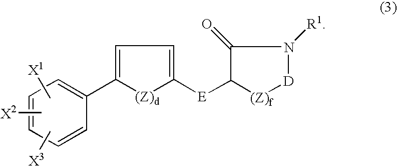
In Formula 3, E and D are each, independently, an sp²- or sp³-hybridized carbon, nitrogen, oxygen or sulfur atom; X¹, X² and X³ are each, independently, a hydrogen atom, C₁-C₆-alkyl, an electronegative atom, such as oxygen, sulfur, fluorine, chlorine, bromine or iodine, or an electronegative functional group, such as aryl, alkylcarbonyl, alkylthiocarbonyl, alkoxycarbonyl, aminocarbonyl, —OH, —CN, —CO₂H, —CO₂C₁-C₆, —[(CH₂)₁₋₆]CO₂H, aryl-CO₂H, aryl-CO₂C₁-C₆, —O[(CH₂)₀₋₆]CH₃, —SO₃H, —SO₂H, —SO₂NH₂, —PO₃H₂, —NO₂, —ONO₂, —CNO, —SH, —CNS, —OSO₃H, —OC(O)(OH), —O—, —S—, halomethyl, dihalomethyl, trihalomethyl or N(R⁴)R⁵, where R⁴ and R⁵ are each, independently, H, aryl, C₁-C₆-alkyl; esters thereof, salts thereof, and any combination thereof, R¹ is a hydrogen atom, C₁-C₆-alkyl, an electronegative atom, such as oxygen, sulfur, fluorine, chlorine, bromine or iodine, or an electronegative functional group, such as aryl, aryl-CO₂H, aryl-CO₂C₁-C₆, alkylcarbonyl, alkylthiocarbonyl, alkoxycarbonyl, aminocarbonyl, —OH, —CN, —CO₂H, —CO₂C₁-C₆, —[(CH₂)₁₋₆]CO₂H, —O[(CH₂)₀₋₆]CH₃, —SO₃H, —SO₂H, —SO₂NH₂, —PO₃H₂, —NO₂, —ONO₂, —CNO, —SH, —CNS, —OSO₃H, —OC(O)(OH), —O—, —S—, halomethyl, dihalomethyl, trihalomethyl or N(R⁴)R⁵, where R⁴ and R⁵ are each, independently, H, aryl, C₁-C₆-alkyl; esters thereof, salts thereof, and any combination thereof; Z is independently selected from the group consisting of C═S, C, O, S, CH, C(O), N, NH, C-alkyl, N-alkyl, C-aryl, N-aryl, N-cycloalkyl and N—P, where P is a carbohydrate moiety, such as a monosaccharide group, for example, a fucosyl, glucosyl, galactosyl, mannosyl, fructosyl, gulosyl, idosyl, talosyl, allosyl, altrosyl, ribosyl, arabinosyl, xylosyl or lyxosyl group; d and f are each 1.

Another preferred compound of Formula 1 is represented below: 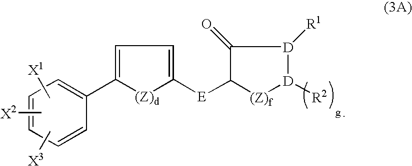
In Formula 3A, E and D are each, independently, an sp²- or sp³-hybridized carbon, nitrogen, oxygen or sulfur atom; X¹, X² and X³ are each, independently, a hydrogen atom, C₁-C₆-alkyl, an electronegative atom, such as oxygen, sulfur, fluorine, chlorine, bromine or iodine, or an electronegative functional group, such as aryl, alkylcarbonyl, alkylthiocarbonyl, alkoxycarbonyl, aminocarbonyl, —OH, —CN, —CO₂H, —CO₂C₁-C₆, —[(CH₂)₁₋₆]CO₂H, aryl-CO₂H, aryl-CO₂C₁-C₆, —O[(CH₂)₀₋₆]CH₃, —SO₃H, —SO₂H, —SO₂NH₂, —PO₃H₂, —NO₂, —ONO₂, —CNO, —SH, —CNS, —OSO₃H, —OC(O)(OH), —O—, —S—, halomethyl, dihalomethyl, trihalomethyl or N(R⁴)R⁵, where R⁴ and R⁵ are each, independently, H, aryl, C₁-C₆-alkyl; esters thereof, salts thereof, and any combination thereof; R¹ and R² are each, independently, selected for the group consisting of a hydrogen atom, C₁-C₆-alkyl, an electronegative atom, such as oxygen, sulfur, fluorine, chlorine, bromine or iodine, or an electronegative functional group, such as aryl, aryl-CO₂H, aryl-CO₂C₁-C₆, alkylcarbonyl, alkylthiocarbonyl, alkoxycarbonyl, aminocarbonyl, —OH, —CN, —CO₂H, —CO₂C₁-C₆, —[(CH₂)₁₋₆]CO₂H, —O[(CH₂)₀₋₆]CH₃, —SO₃H, —SO₂H, —SO₂NH₂, —PO₃H₂, —NO₂, —ONO₂, —CNO, —SH, —CNS, —OSO₃H, —OC(O)(OH), —O—, —S—, halomethyl, dihalomethyl, trihalomethyl and N(R⁴)R⁵, where R⁴ and R⁵ are each, independently, H, aryl, C₁-C₆-alkyl; esters thereof, salts thereof, and any combination thereof; Z is independently selected from the group consisting of C═S, C, O, S, CH, C(O), N, NH, C-alkyl, N-alkyl, C-aryl, N-aryl, N-cycloalkyl and N—P, where P is a carbohydrate moiety, such as a monosaccharide group, for example, a fucosyl, glucosyl, galactosyl, mannosyl, fructosyl, gulosyl, idosyl, talosyl, allosyl, altrosyl, ribosyl, arabinosyl, xylosyl or lyxosyl group; d and f are each 1.

In a preferred embodiment, the invention is directed to a compound of Formula 1, having the Formula 4: 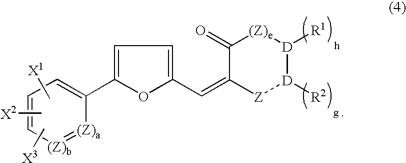
In Formula 4, the dashed line indicates a single or double bond, each D is, independently, an sp²- or sp³-hybridized carbon, nitrogen, oxygen or sulfur atom; X¹, X² and X³ are each, independently, a hydrogen atom, C₁-C₆-alkyl, an electronegative atom, such as oxygen, sulfur, fluorine, chlorine, bromine or iodine, or an electronegative functional group, such as aryl, alkylcarbonyl, alkylthiocarbonyl, alkoxycarbonyl, aminocarbonyl, —OH, —CN, —CO₂H, —CO₂C₁-C₆, —[(CH₂)₁₋₆]CO₂H, —O[(CH₂)₁₋₆]CO₂H, aryl-CO₂H, aryl-CO₂C₁-C₆, —O[(CH₂)₀₋₆]CH₃, —SO₃H, —SO₂H, —SO₂NH₂, —PO₃H₂, —NO₂, —ONO₂, —CNO, —SH, —CNS, —OSO₃H, —OC(O)(OH), —O—, —S—, halomethyl, dihalomethyl, trihalomethyl or N(R⁴)R⁵, wherein R⁴ and R⁵ are each, independently, H, aryl, C₁-C₆-alkyl, esters thereof, salts thereof, and any combination thereof; R¹ and R² are each, independently, a hydrogen atom, C₁-C₆-alkyl, an electronegative atom, such as oxygen, sulfur, fluorine, chlorine, bromine or iodine, or an electronegative functional group, such as aryl, aryl-CO₂H, aryl-CO₂C₁-C₆, alkylcarbonyl, alkylthiocarbonyl, alkoxycarbonyl, aminocarbonyl, —OH, —CN, —CO₂H, —CO₂C₁-C₆, —[(CH₂)₁₋₆]CO₂H, —[(CH₂)₁₋₆]CO₂(CH₂)₀₋₆CH₃, —C₁-C₆—COO—C₁-C₆ (e.g., —CH(CH₃)COOCH₃), —O[(CH₂)₀₋₆]CH₃, —SO₃H, —SO₂H, —SO₂NH₂, —PO₃H₂, —NO₂, —ONO₂, —CNO, —SH, —CNS, —OSO₃H, —OC(O)(OH), —O—, —S—, halomethyl, dihalomethyl, trihalomethyl or N(R⁴)R⁵, where R⁴ and R⁵ are each, independently, H, aryl, or C₁-C₆-alkyl; esters thereof, salts thereof, and any combination thereof; Z is independently selected from the group consisting of C═S, C, O, S, CH, C(O), N, NH, C-alkyl, N-alkyl, C-aryl, N-aryl, N-cycloalkyl and N—P, wherein P is a carbohydrate moiety, such as a monosaccharide group, for example, a fucosyl, glucosyl, galactosyl, mannosyl, fructosyl, gulosyl, idosyl, talosyl, allosyl, altrosyl, ribosyl, arabinosyl, xylosyl or lyxosyl group; and a, b and e, f and g are each 0 or 1.

In a preferred embodiment of Formula 4, X¹, X² and X³ are each, independently, a hydrogen atom, C₁-C₆-alkyl, —OH, —CO₂H, —CO₂C₁-C₆, —[(CH₂)₁₋₆]CO₂H, aryl-CO₂H, aryl-CO₂C₁-C₆, —O[(CH₂)₀₋₆]CH₃, —SO₃H, —SO₂H, —SO₂NH₂, —PO₃H₂, —NO₂, —ONO₂, —CNO, —OSO₃H, —OC(O)(OH), O[(CH₂)₁₋₆]CO₂H, —CO₂(CH₂)₁₋₆H; R¹ and R² are each, independently, a hydrogen atom, C₁-C₆-alkyl, an electronegative atom, such as oxygen, sulfur, fluorine, chlorine, bromine or iodine, or an electronegative functional group, such as aryl, aryl-CO₂H, aryl-CO₂C₁-C₆, alkylcarbonyl, alkylthiocarbonyl, alkoxycarbonyl, aminocarbonyl, —OH, —CN, —CO₂H, —CO₂C₁-C₆, —[(CH₂)₁₋₆]CO₂H, —[(CH₂)₁₋₆]CO₂(CH₂)₀₋₆CH₃, —C₁-C₆—COO—C₁-C₆ (e.g., —CH(CH₃)COOCH₃), —O[(CH₂)₀₋₆]CH₃, —SO₃H, —SO₂H, —SO₂NH₂, —PO₃H₂, —NO₂, —ONO₂, —CNO, —SH, —CNS, —OSO₃H, —OC(O)(OH), —O—, —S—, halomethyl, dihalomethyl, trihalomethyl, and Z is independently selected from the group consisting of C═S, C, O, S, CH, C(O), N, NH, C-alkyl, N-alkyl, C-aryl, N-aryl, and N-cycloalkyl, a and b are 1 and e is 0 or 1.

In another preferred embodiment of Formula 4, X¹, X² and X³ are each, independently, a hydrogen atom, C₁-C₆-alkyl, —OH, —CO₂H, —CO₂C₁-C₆, —[(CH₂)₁₋₆]CO₂H, —SO₃H, —SO₂H, —SO₂NH₂, —PO₃H₂, —NO₂, —O[(CH₂)₁₋₆]CO₂H, or —CO₂(CH₂)₁₋₆H; R¹ and R² are each, independently, a hydrogen atom, C₁-C₆-alkyl, —OH, —CN, —CO₂H, —CO₂C₁-C₆, —[(CH₂)₁₋₆]CO₂H, —[(CH₂)₁₋₆]CO₂(CH₂)₀₋₆CH₃, —C₁-C₆—COO—C₁-C₆ (e.g., —CH(CH₃)COOCH₃), halomethyl, dihalomethyl, or trihalomethyl; Z is independently selected from the group consisting of C═S, S, CH, or C(O), N, NH, C-alkyl, N-alkyl, C-aryl, N-aryl, and N-cycloalkyl, a and b are 1 and e is 0 or 1.

In another preferred embodiment, the invention is directed to a compound of Formula 1, having the Formula 5: 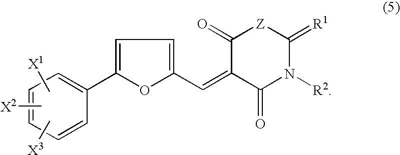
In Formula 5, X¹, X² and X³ are each, independently, a hydrogen atom, C₁-C₆-alkyl, an electronegative atom, such as oxygen, sulfur, fluorine, chlorine, bromine or iodine, or an electronegative functional group, such as aryl, alkylcarbonyl, alkylthiocarbonyl, alkoxycarbonyl, aminocarbonyl, —OH, —CN, —CO₂H, —CO₂C₁-C₆, —[(CH₂)₁₋₆]CO₂H, —O[(CH₂)₁₋₆]CO₂H, aryl-CO₂H, aryl-CO₂C₁-C₆, —O[(CH₂)₀₋₆]CH₃, —SO₃H, —SO₂H, —SO₂NH₂, —PO₃H₂, —NO₂, —ONO₂, —CNO, —SH, —CNS, —OSO₃H, —OC(O)(OH), —O—, —S—, halomethyl, dihalomethyl, trihalomethyl or N(R⁴)R⁵, where R⁴ and R⁵ are each, independently, H, aryl, C₁-C₆-alkyl; esters thereof, salts thereof, and any combination thereof; R¹ and R² are each, independently, a hydrogen atom, C₁-C₆-alkyl, an electronegative atom, such as oxygen, sulfur, fluorine, chlorine, bromine or iodine, or an electronegative functional group, such as aryl, aryl-CO₂H, aryl-CO₂C₁-C₆, alkylcarbonyl, alkylthiocarbonyl, alkoxycarbonyl, aminocarbonyl, —OH, —CN, —CO₂H, —CO₂C₁-C₆, —[(CH₂)₁₋₆]CO₂H, —[(CH₂)₁₋₆]CO₂(CH₂)₀₋₆CH₃, —C₁-C₆—COO—C₁-C₆ (e.g., —CH(CH₃)COOCH₃), —O[(CH₂)₀₋₆]CH₃, —SO₃H, —SO₂H, —SO₂NH₂, —PO₃H₂, —NO₂, —ONO₂, —CNO, —SH, —CNS, —OSO₃H, —OC(O)(OH), —O—, —S—, halomethyl, dihalomethyl, trihalomethyl or N(R⁴)R⁵, where R⁴ and R⁵ are each, independently, H, aryl, C₁-C₆-alkyl; esters thereof, salts thereof, and any combination thereof; and Z is independently selected from the group consisting of C═S, C, O, S, CH, C(O), N, NH, C-alkyl, N-alkyl, C-aryl, N-aryl, N-cycloalkyl and N—P, where P is a carbohydrate moiety, such as a monosaccharide group, for example, a fucosyl, glucosyl, galactosyl, mannosyl, fructosyl, gulosyl, idosyl, talosyl, allosyl, altrosyl, ribosyl, arabinosyl, xylosyl or lyxosyl group.

In a preferred embodiment of Formula 5, X¹, X² and X³ are each, independently, a hydrogen atom, C₁-C₆-alkyl, —OH, —CO₂H, —CO₂C₁-C₆, —[(CH₂)₁₋₆]CO₂H, aryl-CO₂H, aryl-CO₂C₁-C₆, —O[(CH₂)₀₋₆]CH₃, —SO₃H, —SO₂H, —SO₂NH₂, —PO₃H₂, —NO₂, —ONO₂, —CNO, —OSO₃H, —OC(O)(OH), O[(CH₂)₁₋₆]CO₂H, —CO₂(CH₂)₁₋₆H; R¹ and R² are each, independently, a hydrogen atom, C₁-C₆-alkyl, an electronegative atom, such as oxygen, sulfur, fluorine, chlorine, bromine or iodine, or an electronegative functional group, such as aryl, aryl-CO₂H, aryl-CO₂C₁-C₆, alkylcarbonyl, alkylthiocarbonyl, alkoxycarbonyl, aminocarbonyl, —OH, —CN, —CO₂H, —CO₂C₁-C₆, —[(CH₂)₁₋₆]CO₂H, —[(CH₂)₁₋₆]CO₂(CH₂)₀₋₆CH₃, —C₁-C₆—COO—C₁-C₆ (e.g., —CH(CH₃)COOCH₃), —O[(CH₂)₀₋₆]CH₃, —SO₃H, —SO₂H, —SO₂NH₂, —PO₃H₂, —NO₂, —ONO₂, —CNO, —SH, —CNS, —OSO₃H, —OC(O)(OH), —O—, —S—, halomethyl, dihalomethyl, trihalomethyl, and Z is independently selected from the group consisting of C═S, C, O, S, CH, C(O), N, NH, C-alkyl, N-alkyl, C-aryl, N-aryl, and N-cycloalkyl.

In another preferred embodiment of Formula 5, X¹, X² and X³ are each, independently, a hydrogen atom, C₁-C₆-alkyl, —OH, —CO₂H, —CO₂C₁-C₆, —[(CH₂)₁₋₆]CO₂H, —SO₃H, —SO₂H, —SO₂NH₂, —PO₃H₂, —NO₂, O[(CH₂)₁₋₆]CO₂H, or —CO₂(CH₂)₁₋₆H; R¹ is O or S; R² is, independently, a hydrogen atom, C₁-C₆-alkyl, —OH, —CN, —CO₂H, —CO₂C₁-C₆, —[(CH₂)₁₋₆]CO₂H, —[(CH₂)₁₋₆]CO₂(CH₂)₀₋₆CH₃, —C₁-C₆—COO—C₁-C₆ (e.g., —CH(CH₃)COOCH₃), halomethyl, dihalomethyl, or trihalomethyl; Z is independently selected from the group consisting of C═S, S, CH, or C(O), N, NH, C-alkyl, N-alkyl, C-aryl, N-aryl, and N-cycloalkyl.

In another preferred embodiment, the invention is directed to a compound of Formula 1, having the Formula 6: 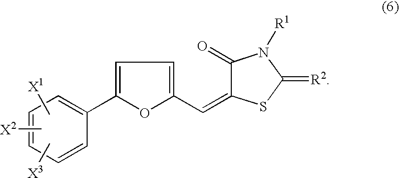
In Formula 6, X¹, X² and X³ are each, independently, a hydrogen atom, C₁-C₆-alkyl, an electronegative atom, such as oxygen, sulfur, fluorine, chlorine, bromine or iodine, or an electronegative functional group, such as aryl, alkylcarbonyl, alkylthiocarbonyl, alkoxycarbonyl, aminocarbonyl, —OH, —CN, —CO₂H, —CO₂C₁-C₆, —[(CH₂)₁₋₆]CO₂H, —O[(CH₂)₁₋₆]CO₂H, aryl-CO₂H, aryl-CO₂C₁-C₆, —CO₂C₁-C₆, —O[(CH₂)₀₋₆]CH₃, —SO₃H, —SO₂H, —SO₂NH₂, —PO₃H₂, —NO₂, —ONO₂, —CNO, —SH, —CNS, —OSO₃H, —OC(O)(OH), —O—, —S—, halomethyl, dihalomethyl, trihalomethyl or N(R⁴)R⁵, where R⁴ and R⁵ are each, independently, H, aryl, C₁-C₆-alkyl; esters thereof, salts thereof, and any combination thereof; R¹ and R² are each, independently, a hydrogen atom, C₁-C₆-alkyl, an electronegative atom, such as oxygen, sulfur, fluorine, chlorine, bromine or iodine, or an electronegative functional group, such as aryl, aryl-CO₂H, aryl-CO₂C₁-C₆, alkylcarbonyl, alkylthiocarbonyl, alkoxycarbonyl, aminocarbonyl, —OH, —CN, —CO₂H, —CO₂C₁-C₆, —[(CH₂)₁₋₆]CO₂H, —[(CH₂)₁₋₆]CO₂(CH₂)₀₋₆CH₃, —C₁-C₆—COO—C₁-C₆ (e.g., —CH(CH₃)COOCH₃), —O[(CH₂)₀₋₆]CH₃, —SO₃H, —SO₂H, —SO₂NH₂, —PO₃H₂, —NO₂, —ONO₂, —CNO, —SH, —CNS, —OSO₃H, —OC(O)(OH), —O—, —S—, halomethyl, dihalomethyl, trihalomethyl or N(R⁴)R⁵, where R⁴ and R⁵ are each, independently, H, aryl, C₁-C₆-alkyl; esters thereof, salts thereof, and any combination thereof; and Z is independently selected from the group consisting of C═S, C, O, S, CH, C(O), N, NH, C-alkyl, N-alkyl, C-aryl, N-aryl, N-cycloalkyl and N—P, where P is a carbohydrate moiety, such as a monosaccharide group, for example, a fucosyl, glucosyl, galactosyl, mannosyl, fructosyl, gulosyl, idosyl, talosyl, allosyl, altrosyl, ribosyl, arabinosyl, xylosyl or lyxosyl group.

In a preferred embodiment of Formula 6, X¹, X² and X³ are each, independently, a hydrogen atom, C₁-C₆-alkyl, —OH, —CO₂H, —CO₂C₁-C₆, —[(CH₂)₁₋₆]CO₂H, aryl-CO₂H, aryl-CO₂C₁-C₆, —O[(CH₂)₀₋₆]CH₃, —SO₃H, —SO₂H, —SO₂NH₂, —PO₃H₂, —NO₂, —ONO₂, —CNO, —OSO₃H, —OC(O)(OH), O[(CH₂)₁₋₆]CO₂H, —CO₂(CH₂)₁₋₆H; R¹ and R² are each, independently, a hydrogen atom, C₁-C₆-alkyl, an electronegative atom, such as oxygen, sulfur, fluorine, chlorine, bromine or iodine, or an electronegative functional group, such as aryl, aryl-CO₂H, aryl-CO₂C₁-C₆, alkylcarbonyl, alkylthiocarbonyl, alkoxycarbonyl, aminocarbonyl, —OH, —CN, —CO₂H, —CO₂C₁-C₆, —[(CH₂)₁₋₆]CO₂H, —[(CH₂)₁₋₆]CO₂(CH₂)₀₋₆CH₃, —C₁-C₆—COO—C₁-C₆ (e.g., —CH(CH₃)COOCH₃), —O[(CH₂)₀₋₆]CH₃, —SO₃H, —SO₂H, —SO₂NH₂, —PO₃H₂, —NO₂, —ONO₂, —CNO, —SH, —CNS, —OSO₃H, —OC(O)(OH), —O—, —S—, halomethyl, dihalomethyl, trihalomethyl, and Z is independently selected from the group consisting of C═S, C, O, S, CH, C(O), N, NH, C-alkyl, N-alkyl, C-aryl, N-aryl, or N-cycloalkyl.

In another preferred embodiment of Formula 6, X¹, X² and X³ are each, independently, a hydrogen atom, C₁-C₆-alkyl, —OH, —CO₂H, —CO₂C₁-C₆, —[(CH₂)₁₋₆]CO₂H, —SO₃H, —SO₂H, —SO₂NH₂, —PO₃H₂, —NO₂, O[(CH₂)₁₋₆]CO₂H, or —CO₂(CH₂)₁₋₆H; R² is O or S; R¹ is, independently, a hydrogen atom, C₁-C₆-alkyl, —OH, —CN, —CO₂H, —CO₂C₁-C₆, —[(CH₂)₁₋₆]CO₂H, —[(CH₂)₁₋₆]CO₂(CH₂)₀₋₆CH₃, —C₁-C₆—COO—C₁-C₆ (e.g., —CH(CH₃)COOCH₃), halomethyl, dihalomethyl, or trihalomethyl; Z is independently selected from the group consisting of C═S, S, CH, or C(O), N, NH, C-alkyl, N-alkyl, C-aryl, N-aryl, and N-cycloalkyl.

It is to be understood that all of the compounds of Formulas 1, 2, 3, 3A, 4, 5 and 6 described above will further include double bonds between adjacent atoms as required to satisfy the valence of each atom. That is, double bonds are added to provide the following number of total bonds to each of the following types of atoms: carbon: four bonds; nitrogen: three bonds; oxygen: two bonds; and sulfur: two bonds.

In a particular embodiment of the invention, the NT/NTR modulator (e.g., NGF/NTR modulator, BDNF/NTR modulator, and NGF/BDNF/NTR modulator) of Formula I is any one of the compounds of Table 1, Table 2, Table 3, and Table 4 or derivatives and fragments thereof, including salts thereof, e.g., pharmaceutically acceptable salts.

In another embodiment, the invention pertains to the NT/NTR modulators (e.g., NGF/NTR modulators, BDNF/NTR modulators, and NGF/BDNF/NTR modulators) of Formula 1, Formula 2, Formula 3, Formula 3A, Formula 4, Formula 5, and Formula 6 described herein, including salts thereof, e.g., pharmaceutically acceptable salts. Particular embodiments of the invention pertain to the modulating compounds of Table 1, Table 2, Table 3, and Table 4 or derivatives thereof, including salts thereof, e.g., pharmaceutically acceptable salts. In a preferred embodiment of the invention, exemplary BDNF/NTR modulators are shown in Table 1, exemplary NGF/BDNF/NTR modulators are shown in Table 2, and exemplary NGF/NTR modulators are shown in Table 3.

In yet another embodiment, the invention pertains to pharmaceutical compositions comprising NT/NTR modulating compounds described herein and a pharmaceutical acceptable carrier.

In another embodiment, the invention includes any novel compound or pharmaceutical compositions containing compounds of the invention described herein. For example, compounds and pharmaceutical compositions containing compounds set forth herein (e.g., Table 1, Table 2, Table 3 and Table 4) are part of this invention, including salts thereof, e.g., pharmaceutically acceptable salts.

Particular compounds of the invention also include the following compounds, numbered 1-143, each of which is considered a separate embodiment of the invention. Moreover, the listing of the compounds below is intended merely as a convenience, and not necessarily as imparting the characteristic of a group. A key describing the representations of the biological activity of the compounds (“XXXX,” “XXX,” etc.) follows each table.

TABLE 1

| | | | BDNF | NGF |
|---|---|---|---|---|
| | | | Activity | Activity |
| | | | % Maximal | % Maximal |
| Compound | Structure | Mol Wt. | Binding | Binding |
| 1 | 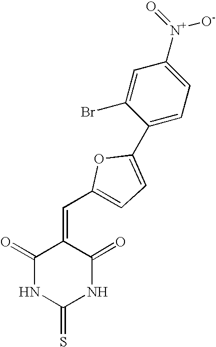 | 422.22 | XXXXX | * |
| | 5-[5-(2-Bromo-4-nitrophenyl)furan-2- | | | |
| | ylmethylene]-2-thioxo-dihydro-pyrimidine- | | | |
| | 4,6-dione | | | |
| 2 | 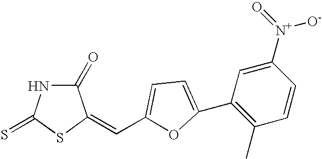 | 346.38 | XXXXX | * |
| | 5-[5-(2-Methyl-5-nitrophenyl)furan-2- | | | |
| | ylmethylene]-2-thioxo-thiazolidin-4-one | | | |
| 3 | 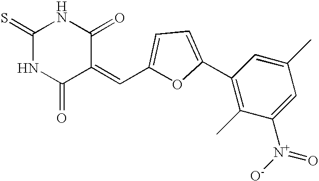 | 371.37 | XXXXX | * |
| | 5-[5-(2,5-Dimethyl-3-nitrophenyl)furan-2- | | | |
| | ylmethylene]-2-thioxo-dihydro-pyrimidine- | | | |
| | 4,6-dione | | | |
| 4 | 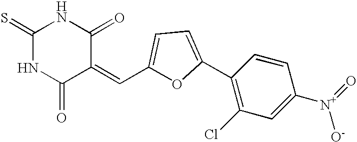 | XXXXX | * | |
| | 5-[5-(2-Chloro-4-nitrophenyl)furan-2- | | | |
| | ylmethylene]-2-thioxo-dihydro-pyrimidine- | | | |
| | 4,6-dione | | | |
| 5 | 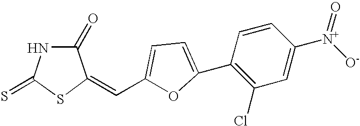 | 366.80 | XXXXX | * |
| | 5-[5-(2-Chloro-4-nitrophenyl)furan-2- | | | |
| | ylmethylene]-2-thioxo-thiazolidin-4-one | | | |
| 6 | 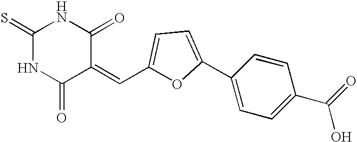 | 342.33 | XXXXX | * |
| | 4-[5-(4,6-Dioxo-2-thioxo-tetrahydro- | | | |
| | pyrimidin-5-ylidenemethyl)furan-2-yl]benzoic | | | |
| | acid | | | |
| 7 | 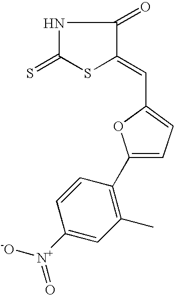 | 346.39 | XXXXX | * |
| | 5-[5-(2-Methyl-4-nitrophenyl)furan-2- | | | |
| | ylmethylene]-2-thioxo-thiazolidin-4-one | | | |
| 8 | 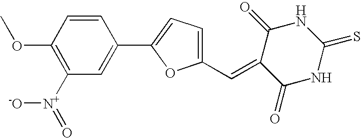 | 373.35 | XXXXX | * |
| | 5-[5-(4-Methoxy-3-nitrophenyl)furan-2- | | | |
| | ylmethylene]-2-thioxo-dihydro-pyrimidine- | | | |
| | 4,6-dione | | | |
| 9 | 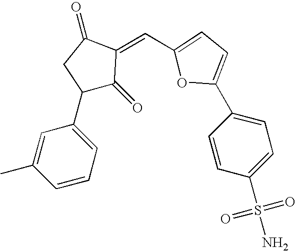 | 423.45 | XXXXX | * |
| | 4-[5-(3,5-Dioxo-1-m-tolyl-pyrazolidin-4- | | | |
| | ylidenemethyl)furan-2-yl]benzenesulfonamide | | | |
| 10 | 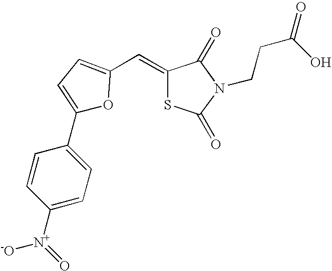 | 404.42 | XXXXX | * |
| | 3-{5-[5-(4-Nitrophenyl)furan-2-ylmethylene]- | | | |
| | 4-oxo-2-thioxo-thiazolidin-3-yl}-propionic | | | |
| | acid | | | |
| 11 | 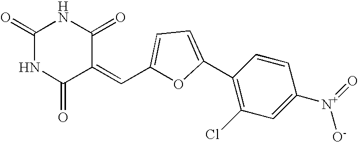 | 361.70 | XXXXX | * |
| | 5-[5-(2-Chloro-4-nitrophenyl)furan-2- | | | |
| | ylmethylene]-pyrimidine-2,4,6-trione | | | |
| 12 | 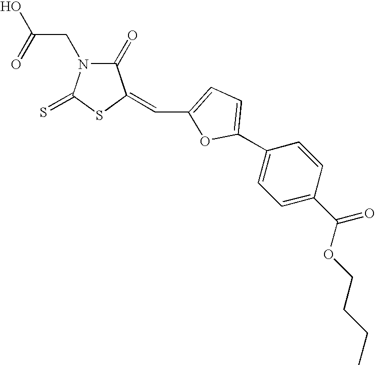 | 445.52 | XXXXX | * |
| | 4-[5-(3-Carboxymethyl-4-oxo-2-thioxo- | | | |
| | thiazolidin-5-ylidenemethyl)furan-2- | | | |
| | yl]benzoic acid butyl ester | | | |
| 13 | 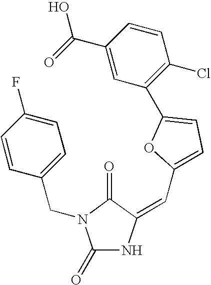 | 440.82 | XXXXX | * |
| | 4-Chloro-3-{5-[1-(4-fluorobenzyl)-2,5-dioxo- | | | |
| | imidazolidin-4-ylidenemethyl]furan-2- | | | |
| | yl}benzoic acid | | | |
| 14 | 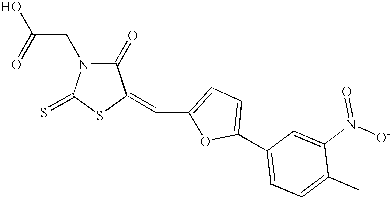 | 404.42 | XXXXX | * |
| | {5-[5-(4-Methyl-3-nitrophenyl)furan-2- | | | |
| | ylmethylene]-4-oxo-2-thioxo-thiazolidin-3- | | | |
| | yl}-acetic acid | | | |
| 15 | 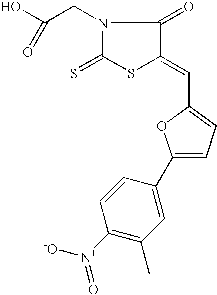 | 404.42 | XXXXX | |
| | {5-[5-(3-Methyl-4-nitrophenyl)furan-2- | | | |
| | ylmethylene]-4-oxo-2-thioxo-thiazolidin-3- | | | |
| | yl}-acetic acid | | | |
| 16 | 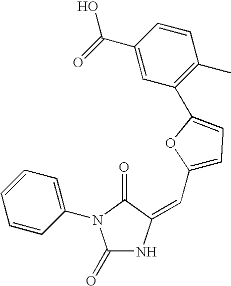 | 388.38 | XXXXX | * |
| | 3-[5-(2,5-Dioxo-1-phenyl-imidazolidin-4- | | | |
| | ylidenemethyl)furan-2-yl]-4-methylbenzoic | | | |
| | acid | | | |
| 17 | 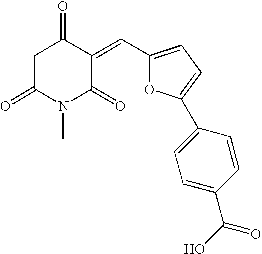 | 340.29 | XXXXX | * |
| | 4-[5-(1-Methyl-2,4,6-trioxo-tetrahydro- | | | |
| | pyrimidin-5-ylidenemethyl)furan-2-yl]benzoic | | | |
| | acid | | | |
| 18 | 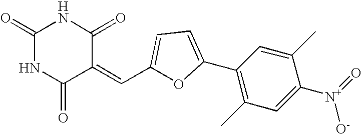 | 355.30 | XXXXX | * |
| | 5-[5-(2,5-Dimethyl-4-nitrophenyl)furan-2- | | | |
| | ylmethylene]-pyrimidine-2,4,6-trione | | | |
| 19 | 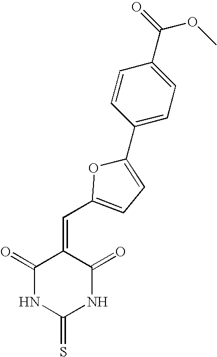 | 356.36 | XXXXX | * |
| | 4-[5-(4,6-Dioxo-2-thioxo-tetrahydro- | | | |
| | pyrimidin-5-ylidenemethyl)furan-2-yl]benzoic | | | |
| | acid methyl ester | | | |
| 20 | 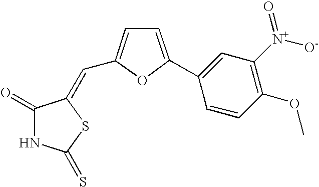 | 362.38 | XXXXX | * |
| | 5-[5-(4-Methoxy-3-nitrophenyl)furan-2- | | | |
| | ylmethylene]-2-thioxo-thiazolidin-4-one | | | |
| 21 | 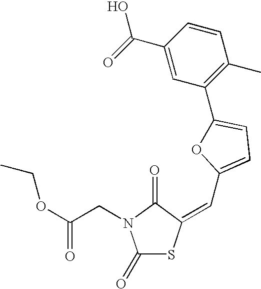 | 415.42 | XXXXX | * |
| | 3-[5-(3-Ethoxycarbonylmethyl-2,4-dioxo- | | | |
| | thiazolidin-5-ylidenemethyl)furan-2-yl]-4- | | | |
| | methylbenzoic acid | | | |
| 22 | 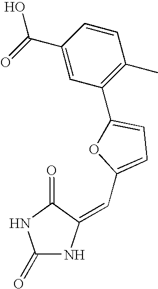 | 312.28 | XXXXX | * |
| | 3-[5-(2,5-Dioxo-imidazolidin-4- | | | |
| | ylidenemethyl)furan-2-yl]-4-methylbenzoic | | | |
| | acid | | | |
| 23 | 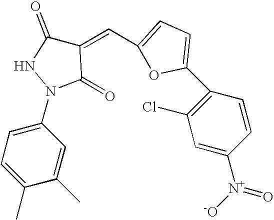 | 437.84 | XXXXX | * |
| | 4-[5-(2-Chloro-4-nitrophenyl)furan-2- | | | |
| | ylmethylene]-1-(3,4-dimethylphenyl)- | | | |
| | pyrazolidine-3,5-dione | | | |
| 24 | 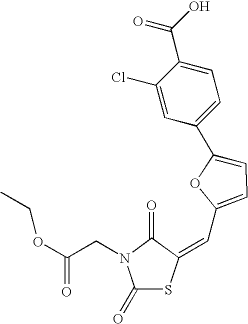 | 435.84 | XXXXX | * |
| | 2-Chloro-4-[5-(3-ethoxycarbonylmethyl-2,4- | | | |
| | dioxo-thiazolidin-5-ylidenemethyl)furan-2- | | | |
| | yl]benzoic acid | | | |
| 25 | 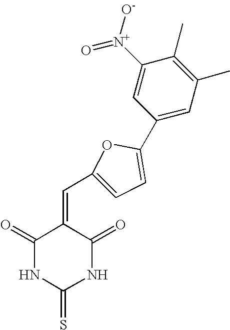 | 371.37 | XXXXX | * |
| | 5-[5-(3,4-Dimethyl-5-nitrophenyl)furan-2- | | | |
| | ylmethylene]-2-thioxo-dihydro-pyrimidine- | | | |
| | 4,6-dione | | | |
| 26 | 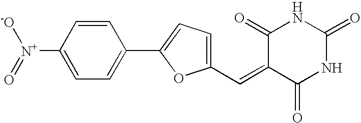 | 327.26 | XXXXX | * |
| | 5-[5-(4-Nitrophenyl)furan-2-ylmethylene]- | | | |
| | pyrimidine-2,4,6-trione | | | |
| 27 | 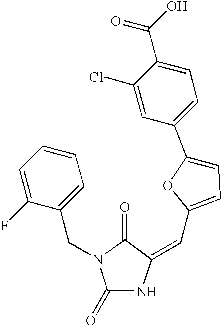 | 440.82 | XXXXX | * |
| | 2-Chloro-4-{5-[1-(2-fluorobenzyl)-2,5-dioxo- | | | |
| | imidazolidin-4-ylidenemethyl]furan-2- | | | |
| | yl}benzoic acid | | | |
| 28 | 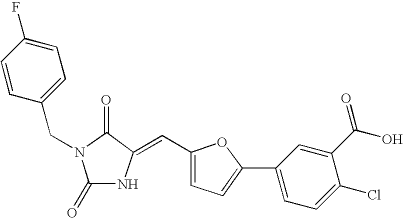 | 440.82 | XXXX | * |
| | 2-Chloro-5-{5-[1-(4-fluorobenzyl)-2,5-dioxo- | | | |
| | imidazolidin-4-ylidenemethyl]furan-2- | | | |
| | yl}benzoic acid | | | |
| 29 | 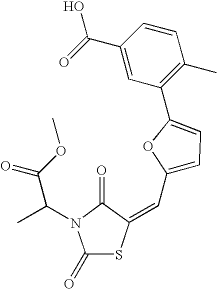 | 415.42 | XXXX | * |
| | 3-{5-[3-(1-Methoxycarbonyl-ethyl)-2,4- | | | |
| | dioxo-thiazolidin-5-ylidenemethyl]furan-2- | | | |
| | yl}-4-methylbenzoic acid | | | |
| 30 | 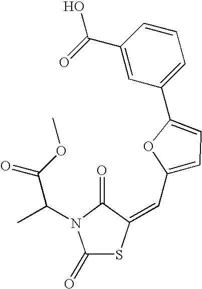 | 435.84 | XXXX | * |
| | 2-Chloro-4-{5-[3-(1-methoxycarbonyl-ethyl)- | | | |
| | 2,4-dioxo-thiazolidin-5-ylidenemethyl]furan- | | | |
| | 2-yl}benzoic acid | | | |
| 31 | 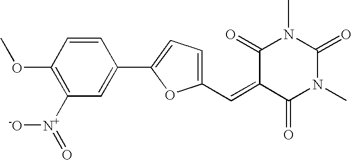 | 385.33 | XXXX | * |
| | 5-[5-(4-Methoxy-3-nitrophenyl)furan-2- | | | |
| | ylmethylene]-1,3-dimethyl-pyrimidine-2,4,6- | | | |
| | trione | | | |
| 32 | 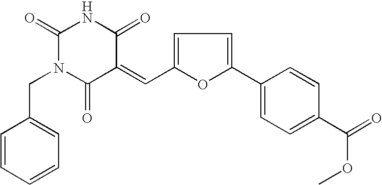 | 430.42 | XXXX | * |
| | 4-[5-(1-benzyl-2,4,6-trioxo-tetrahydro- | | | |
| | pyrimidin-5-ylidenemethyl)furan-2-yl]benzoic | | | |
| | acid methyl ester | | | |
| 33 | 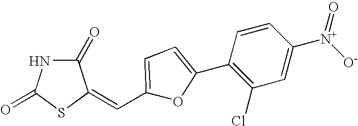 | 350.74 | XXXX | * |
| | 5-[5-(2-Chloro-4-nitrophenyl)furan-2- | | | |
| | ylmethylene]-thiazolidine-2,4-dione | | | |
| 34 | 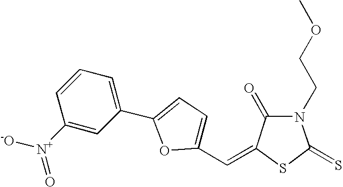 | 390.44 | XXXX | * |
| | 3-(2-Methoxy-ethyl)-5-[5-(3- | | | |
| | nitrophenyl)furan-2-ylmethylene]-2-thioxo- | | | |
| | thiazolidin-4-one | | | |
| 35 | 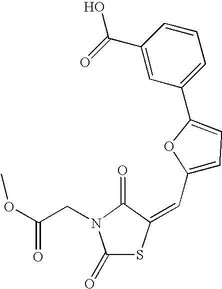 | 401.34 | XXXX | * |
| | 4-[5-(3-Methoxycarbonylmethyl-2,4-dioxo- | | | |
| | thiazolidin-5-ylidenemethyl)furan-2-yl]-3- | | | |
| | methylbenzoic acid | | | |
| 36 | 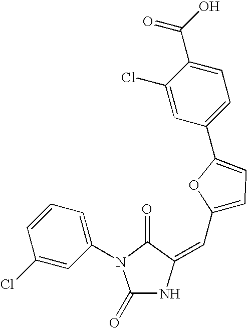 | 443.25 | XXXX | * |
| | 2-Chloro-4-{5-[1-(3-chlorophenyl)-2,5-dioxo- | | | |
| | imidazolidin-4-ylidenemethyl]furan-2- | | | |
| | yl}benzoic acid | | | |
| 37 | 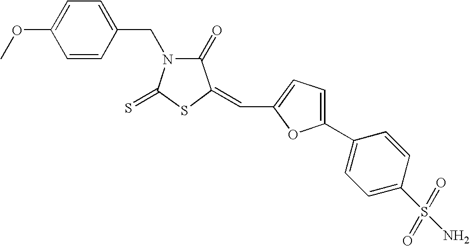 | 486.59 | XXX | * |
| | 4-{5-[3-(4-Methoxybenzyl)-4-oxo-2-thioxo- | | | |
| | thiazolidin-5-ylidenemethyl]furan-2- | | | |
| | yl}benzenesulfonamide | | | |
| 38 | 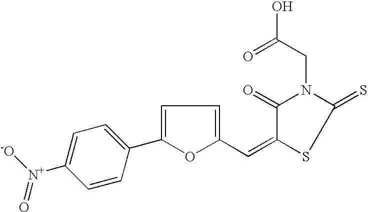 | 390.40 | XXX | * |
| | {5-[5-(4-Nitrophenyl)furan-2-ylmethylene]-4- | | | |
| | oxo-2-thioxo-thiazolidin-3-yl}-acetic acid | | | |
| 39 | 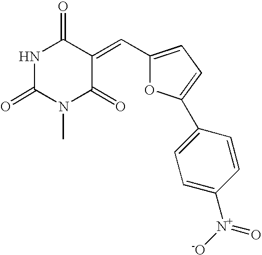 | 341.28 | XXX | * |
| | 1-Methyl-5-[5-(4-nitrophenyl)furan-2- | | | |
| | ylmethylene]-pyrimidine-2,4,6-trione | | | |
| 40 | 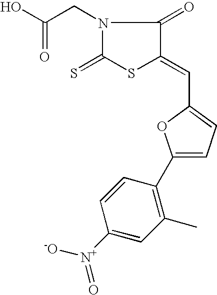 | 404.43 | XXX | * |
| | {5-[5-(2-Methyl-4-nitrophenyl)furan-2- | | | |
| | ylmethylene]-4-oxo-2-thioxo-thiazolidin-3- | | | |
| | yl}-acetic acid | | | |
| 41 | 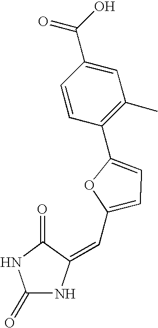 | 312.28 | XXX | * |
| | 4-[5-(2,5-Dioxo-imidazolidin-4- | | | |
| | ylidenemethyl)furan-2-yl]-3-methylbenzoic | | | |
| | acid | | | |
| 42 | 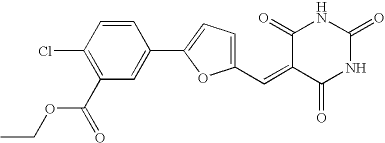 | 416.82 | XXX | * |
| | 2-Chloro-5-[5-(2,4,6-trioxo-tetrahydro- | | | |
| | pyrimidin-5-ylidenemethyl)furan-2-yl]benzoic | | | |
| | acid butyl ester | | | |
| 43 | 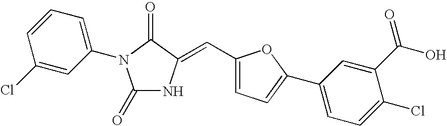 | 443.24 | XX | * |
| | 2-Chloro-5-{5-[1-(3-chlorophenyl)-2,5-dioxo- | | | |
| | imidazolidin-4-ylidenemethyl]furan-2- | | | |
| | yl}benzoic acid | | | |
| 44 | 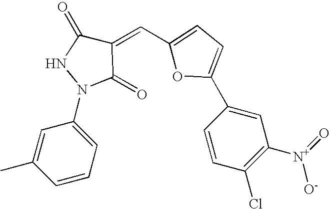 | 423.82 | XX | * |
| | 4-[5-(4-Chloro-3-nitrophenyl)furan-2- | | | |
| | ylmethylene]-1-m-tolyl-pyrazolidine-3,5- | | | |
| | dione | | | |
| 45 | 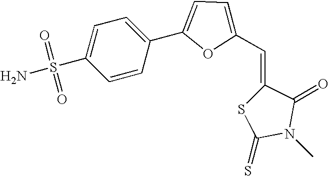 | 380.46 | XX | * |
| | 4-[5-(3-Methyl-4-oxo-2-thioxo-thiazolidin-5- | | | |
| | ylidenemethyl)furan-2-yl]benzenesulfonamide | | | |
| 46 | 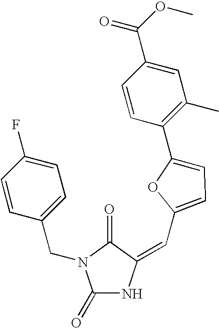 | 434.43 | XX | * |
| | 4-{5-[1-(4-Fluorobenzyl)-2,5-dioxo- | | | |
| | imidazolidin-4-ylidenemethyl]furan-2-yl}-3- | | | |
| | methylbenzoic acid methyl ester | | | |
| 47 | 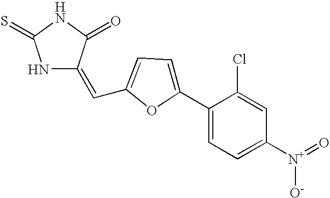 | 349.75 | XX | * |
| | 5-[5-(2-Chloro-4-nitrophenyl)furan-2- | | | |
| | ylmethylene]-2-thioxo-imidazolidin-4-one | | | |
| 48 | 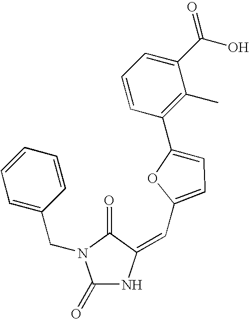 | 388.38 | XX | * |
| | 3-[5-(2,5-Dioxo-1-phenyl-imidazolidin-4- | | | |
| | ylidenemethyl)furan-2-yl]-2-methylbenzoic | | | |
| | acid | | | |
| 49 | 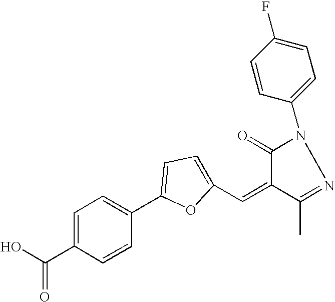 | 390.37 | XX | * |
| | 4-{5-[1-(4-Fluorophenyl)-3-methyl-5-oxo- | | | |
| | 1,5-dihydro-pyrazol-4-ylidenemethyl]furan-2- | | | |
| | yl}benzoic acid | | | |
| 50 | 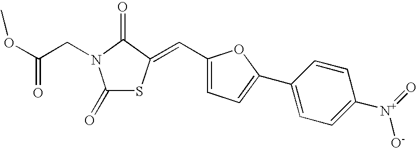 | 388.36 | XX | * |
| | {5-[5-(4-Nitrophenyl)furan-2-ylmethylene]- | | | |
| | 2,4-dioxo-thiazolidin-3-yl}-acetic acid methyl | | | |
| | ester | | | |
| 51 | 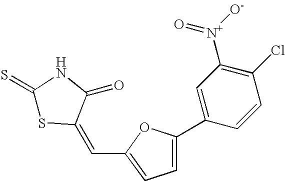 | 366.80 | XX | * |
| | 5-[5-(4-Chloro-3-nitrophenyl)furan-2- | | | |
| | ylmethylene]-2-thioxo-thiazolidin-4-one | | | |
| 52 | 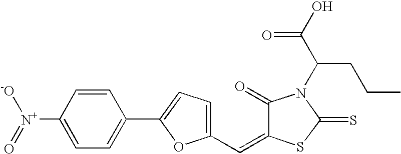 | 432.47 | XX | * |
| | 2-{5-[5-(4-Nitrophenyl)furan-2-ylmethylene]- | | | |
| | 4-oxo-2-thioxo-thiazolidin-3-yl}-pentanoic | | | |
| | acid | | | |
| 53 | 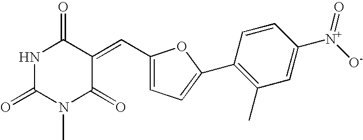 | 355.31 | XX | * |
| | 1-Methyl-5-[5-(2-methyl-4-nitrophenyl)furan- | | | |
| | 2-ylmethylene]-pyrimidine-2,4,6-trione | | | |
| 54 | 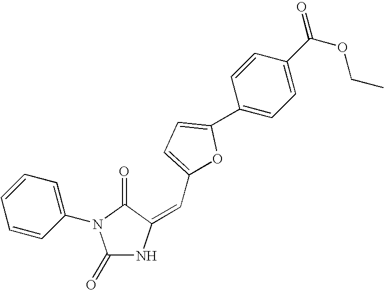 | 402.41 | XX | * |
| | 4-[5-(2,5-Dioxo-1-phenyl-imidazolidin-4- | | | |
| | ylidenemethyl)furan-2-yl]benzoic acid ethyl | | | |
| | ester | | | |
| 55 | 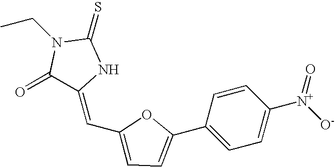 | 343.36 | XX | * |
| | 3-Ethyl-5-[5-(4-nitrophenyl)furan-2- | | | |
| | ylmethylene]-2-thioxo-imidazolidin-4-one | | | |
| 56 | 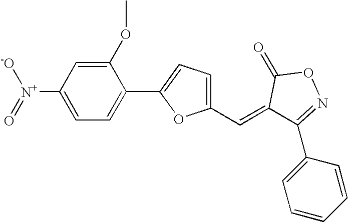 | 390.35 | X | * |
| | 4-[5-(2-Methoxy-4-nitrophenyl)furan-2- | | | |
| | ylmethylene]-3-phenyl-4H-isoxazol-5-one | | | |
| 57 | 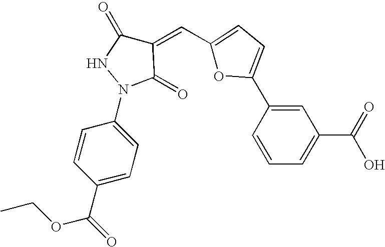 | 446.42 | X | * |
| | 3-{5-[3,5-Dioxo-1-phenyl-(4-ethyl ester)- | | | |
| | pyrazolidin-4-ylidenemethyl]furan-2- | | | |
| | yl}benzoic acid | | | |
| 58 | 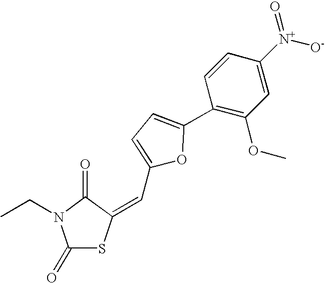 | 374.37 | X | * |
| | 3-Ethyl-5-[5-(2-methoxy-4-nitrophenyl)furan- | | | |
| | 2-ylmethylene]-thiazolidine-2,4-dione | | | |
| 59 | 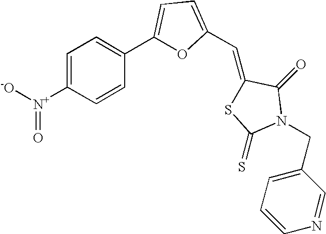 | 423.47 | X | * |
| | 5-[5-(4-Nitrophenyl)furan-2-ylmethylene]-3- | | | |
| | pyridin-3-ylmethyl-2-thioxo-thiazolidin-4-one | | | |
| 60 | 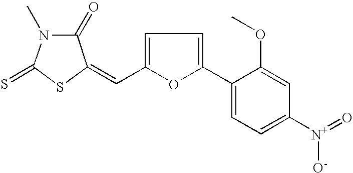 | 376.41 | X | * |
| | 5-[5-(2-Methoxy-4-nitrophenyl)furan-2- | | | |
| | ylmethylene]-3-methyl-2-thioxo-thiazolidin-4- | | | |
| | one | | | |
| 61 | 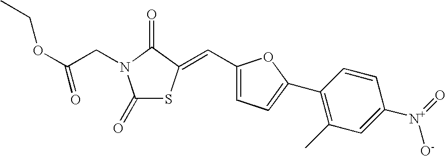 | 416.41 | X | * |
| | {5-[5-(2-Methyl-4-nitrophenyl)furan-2- | | | |
| | ylmethylene]-2,4-dioxo-thiazolidin-3-yl}- | | | |
| | acetic acid ethyl ester | | | |
| 62 | 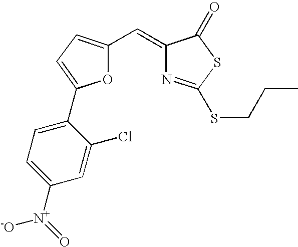 | 408.89 | X | * |
| | 4-[5-(2-Chloro-4-nitrophenyl)furan-2- | | | |
| | ylmethylene]-2-propylsulfanyl-4H-thiazol-5- | | | |
| | one | | | |
| 63 | 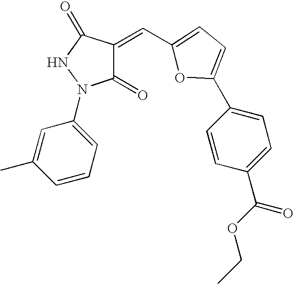 | 416.43 | X * | |
| | 4-[5-(3,5-Dioxo-1-m-tolyl-pyrazolidin-4- | | | |
| | ylidenemethyl)furan-2-yl]benzoic acid ethyl | | | |
| | ester | | | |
| 64 | 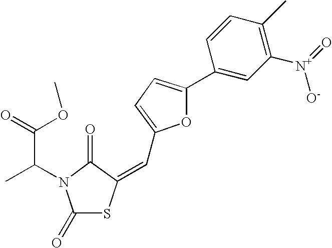 | 416.41 | X | * |
| | 2-{5-[5-(4-Methyl-3-nitrophenyl)furan-2- | | | |
| | ylmethylene]-2,4-dioxo-thiazolidin-3-yl}- | | | |
| | propionic acid methyl ester | | | |

% MAXIMAL BINDING KEY
0 < XXXXX < 10
10 < XXXX < 20
20 < XXX < 30
30 < XX < 40
40 < 50
50 < *

TABLE 2

| | | | BDNF | NGF |
|---|---|---|---|---|
| | | | Activity | Activity |
| | | | % Maximal | % Maximal |
| Compound | Structure | Mol Wt. | Binding | Binding |
| 65 | 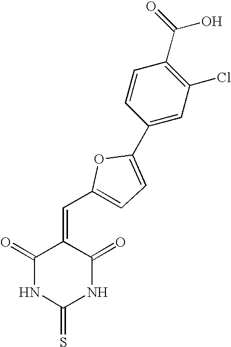 | 376.78 | XXXXX | XXX |
| | 2-Chloro-4-[5-(4,6-dioxo-2-thioxo- | | | |
| | tetrahydro-pyrimidin-5- | | | |
| | ylidenemethyl)furan-2-yl]benzoic acid | | | |
| 66 | 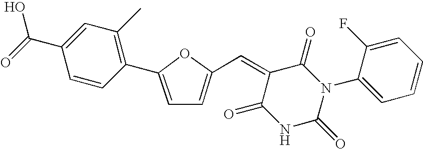 | 434.38 | XXXXX | XXX |
| | 4-{5-[1-(2-Fluorophenyl)-2,4,6-trioxo- | | | |
| | tetrahydro-pyrimidin-5- | | | |
| | ylidenemethyl]furan-2-yl}-3- | | | |
| | methylbenzoic acid | | | |
| 67 | 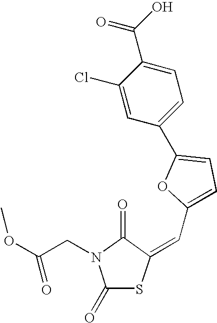 | 421.82 | XXXXX | XX |
| | 2-Chloro-4-[5-(3- | | | |
| | methoxycarbonylmethyl-2,4-dioxo- | | | |
| | thiazolidin-5-ylidenemethyl)furan-2- | | | |
| | yl]benzoic acid | | | |
| 68 | 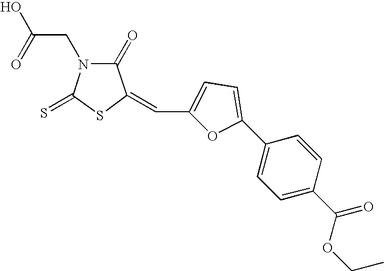 | 417.46 | XXXXX | XX |
| | 4-[5-(3-Carboxymethyl-4-oxo-2-thioxo- | | | |
| | thiazolidin-5-ylidenemethyl)furan-2- | | | |
| | yl]benzoic acid ethyl ester | | | |
| 69 | 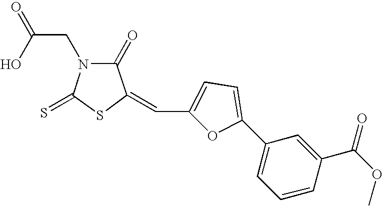 | 403.43 | XXXXX | XX |
| | 3-[5-(3-Carboxymethyl-4-oxo-2-thioxo- | | | |
| | thiazolidin-5-ylidenemethyl)furan-2- | | | |
| | yl]benzoic acid methyl ester | | | |
| 70 | 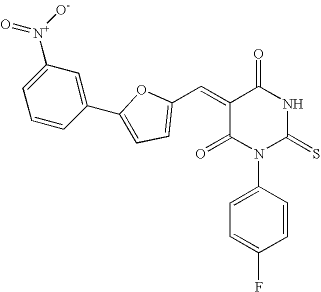 | 437.41 | XXXXX | X |
| | 1-(4-Fluorophenyl)-5-[5-(3- | | | |
| | nitrophenyl)furan-2-ylmethylene]-2- | | | |
| | thioxo-dihydro-pyrimidine-4,6-dione | | | |
| 71 | 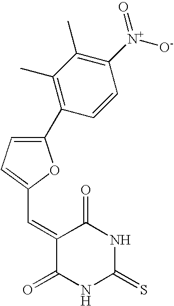 | 371.37 | XXXXX | X |
| | 5-[5-(2,3-Dimethyl-4-nitrophenyl)furan- | | | |
| | 2-ylmethylene]-2-thioxo-dihydro- | | | |
| | pyrimidine-4,6-dione | | | |
| 72 | 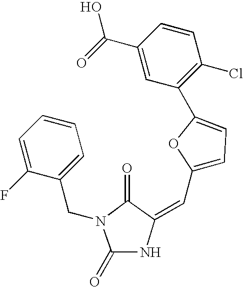 | 440.81 | XXXXX | X |
| | 4-Chloro-3-{5-[1-(2-fluorobenzyl)-2,5- | | | |
| | dioxo-imidazolidin-4- | | | |
| | ylidenemethyl]furan-2-yl}benzoic acid | | | |
| 73 | 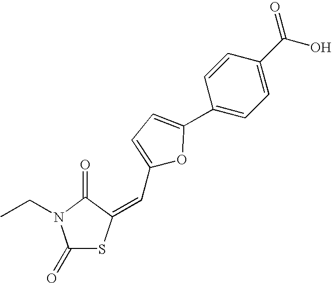 | 343.36 | XXXXX | X |
| | 4-[5-(3-Ethyl-2,4-dioxo-thiazolidin-5- | | | |
| | ylidenemethyl)furan-2-yl]benzoic acid | | | |
| 74 | 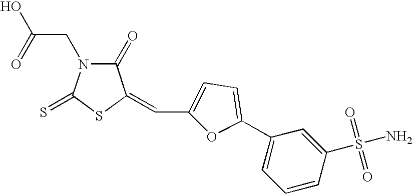 | 424.47 | XXXXX | X |
| | {4-oxo-5-[5-(3-sulfamoylphenyl)furan-2- | | | |
| | ylmethylene]-2-thioxo-thiazolidin-3-yl}- | | | |
| | acetic acid | | | |
| 75 | 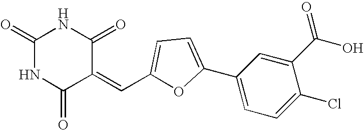 | 360.71 | XXXXX | X |
| | 2-Chloro-5-[5-(2,4,6-trioxo-tetrahydro- | | | |
| | pyrimidin-5-ylidenemethyl)furan-2- | | | |
| | yl]benzoic acid | | | |
| 76 | 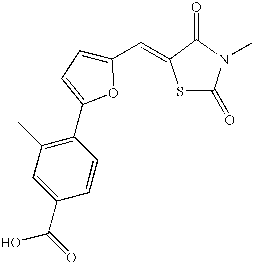 | 343.36 | XXXXX | X |
| | 3-Methyl-4-[5-(3-methyl-2,4-dioxo- | | | |
| | thiazolidin-5-ylidenemethyl)furan-2- | | | |
| | yl]benzoic acid | | | |
| 77 | 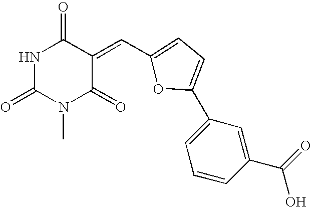 | 340.29 | XXXX | XXX |
| | 3-[5-(1-Methyl-2,4,6-trioxo-tetrahydro- | | | |
| | pyrimidin-5-ylidenemethyl)furan-2- | | | |
| | yl]benzoic acid | | | |
| 78 | 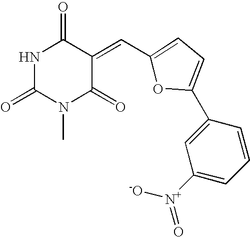 | 341.28 | XXXX | X |
| | 1-Methyl-5-[5-(3-nitrophenyl)furan-2- | | | |
| | ylmethylene]-pyrimidine-2,4,6-trione | | | |
| 79 | 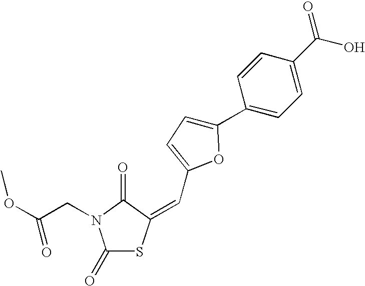 | 387.37 | XXXX | X |
| | 4-[5-(3-Methoxycarbonylmethyl-2,4- | | | |
| | dioxo-thiazolidin-5-ylidenemethyl)furan- | | | |
| | 2-yl]benzoic acid | | | |
| 80 | 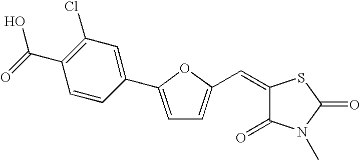 | 363.78 | XXXX | X |
| | 2-Chloro-4-[5-(3-methyl-2,4-dioxo- | | | |
| | thiazolidin-5-ylidenemethyl)furan-2- | | | |
| | yl]benzoic acid | | | |
| 81 | 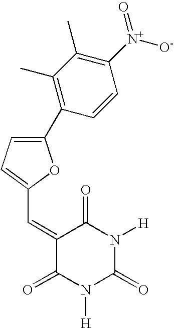 | 355.31 | XXX | XX |
| | 5-[5-(2,3-Dimethyl-4-nitrophenyl)furan- | | | |
| | 2-ylmethylene]-pyrimidine-2,4,6-trione | | | |
| 82 | 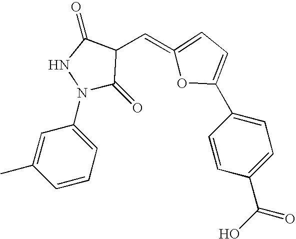 | 388.38 | XXX | X |
| | 4-[5-(3,5-Dioxo-1-m-tolyl-pyrazolidin-4- | | | |
| | ylidenemethyl)furan-2-yl]benzoic acid | | | |
| 83 | 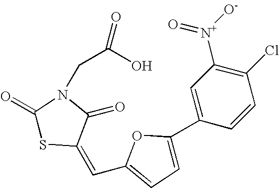 | 424.84 | XX | X |
| | {5-[5-(4-Chloro-3-nitrophenyl)furan-2- | | | |
| | ylmethylene]-4-oxo-2-thioxo-thiazolidin- | | | |
| | 3-yl}-acetic acid | | | |
| 84 | 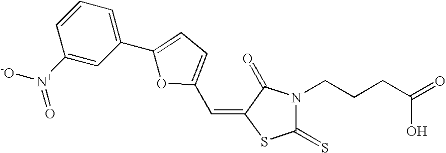 | 418.45 | X | XXXXX |
| | 4-{5-[5-(3-Nitrophenyl)furan-2- | | | |
| | ylmethylene]-4-oxo-2-thioxo-thiazolidin- | | | |
| | 3-yl}-butyric acid | | | |
| Com- pound A | 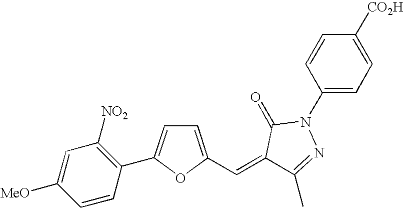 | 447.40 | No data | No data |
| | 4-{4-[5-(4-Methoxy-2-nitro-phenyl)- | | | |
| | furan-2-ylmethylene]-3-methyl-5-oxo- | | | |
| | 4,5-dihydro-pyrazol-1-yl}-benzoic acid | | | |
| Com- pound B | 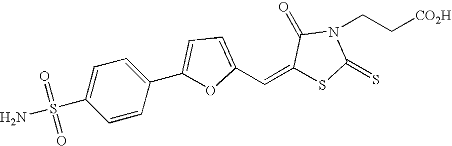 | 438.50 | No data | No data |
| | 3-{4-oxo-5-[5-(4-sulfamoylphenyl)furan- | | | |
| | 2-ylmethylene]-2-thioxo-thiazolidin-3-yl- | | | |
| | propionic acid | | | |

% MAXIMAL BINDING KEY
0 < XXXXX < 10
10 < XXXX < 20
20 < XXX < 30
30 < XX < 40
40 < X < 50
50 < *

TABLE 3

| | | | BDNF | NGF |
|---|---|---|---|---|
| | | | Activity | Activity |
| | | | % Maximal | % Maximal |
| Compound | Structure | Mol Wt. | Binding | Binding |
| 85 | 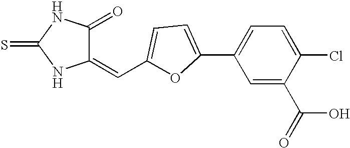 | 348.77 | * | X |
| | 2-Chloro-5-[5-(5-oxo-2-thioxo- | | | |
| | imidazolidin-4-ylidenemethyl)furan-2- | | | |
| | yl]benzoic_acid | | | |

% MAXIMAL BINDING KEY
0 < XXXXX < 10
10 < XXXX < 20
20 < XXX < 30
30 < XX < 40
40 < X < 50
50 < *

TABLE 4

| | | | BDNF | NGF |
|---|---|---|---|---|
| | | | Activity | Activity |
| | | | % Maximal | % Maximal |
| Compound | Structure | Mol Wt. | Binding | Binding |
| 86 | 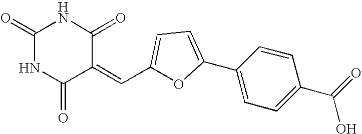 | 326.27 | No Data | No Data |
| | 4-[5-(2,4,6-Trioxo-tetrahydro-pyrimidin- | | | |
| | 5-ylidenemethyl)furan-2-yl]benzoic acid | | | |
| 87 | 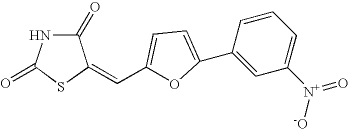 | 316.30 | * | * |
| | 5-[5-(3-Nitrophenyl)furan-2- | | | |
| | ylmethylene]-thiazolidine-2,4-dione | | | |
| 88 | 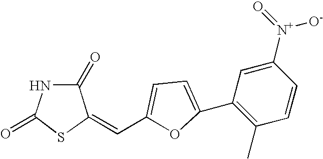 | 330.32 | * | * |
| | 5-[5-(2-Methyl-5-nitrophenyl)furan-2- | | | |
| | ylmethylene]-thiazolidine-2,4-dione | | | |
| 89 | 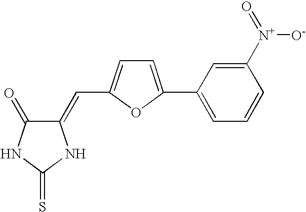 | 315.31 | * | * |
| | 5-[5-(3-Nitrophenyl)furan-2- | | | |
| | ylmethylene]-2-thioxo-imidazolidin-4- | | | |
| | one | | | |
| 90 | 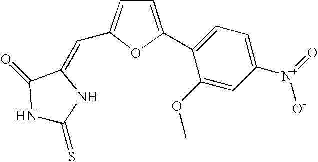 | 345.34 | * | * |
| | 5-[5-(2-Methoxy-4-nitrophenyl)furan-2- | | | |
| | ylmethylene]-2-thioxo-imidazolidin-4- | | | |
| | one | | | |
| 91 | 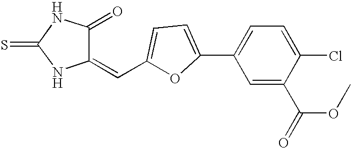 | 362.80 | * | * |
| | 2-Chloro-5-[5-(5-oxo-2-thioxo- | | | |
| | imidazolidin-4-ylidenemethyl)furan-2- | | | |
| | yl]benzoic acid methyl ester | | | |
| 92 | 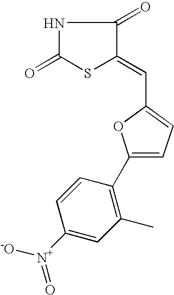 | 330.32 | * | * |
| | 5-[5-(2-Methyl-4-nitrophenyl)furan-2- | | | |
| | ylmethylene]-thiazolidine-2,4-dione | | | |
| 93 | 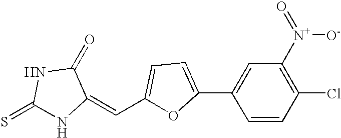 | 349.75 | * | * |
| | 5-[5-(4-Chloro-3-nitrophenyl)furan-2- | | | |
| | ylmethylene]-2-thioxo-imidazolidin-4- | | | |
| | one | | | |
| 94 | 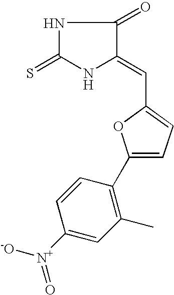 | 329.33 | * | * |
| | 5-[5-(2-Methyl-4-nitrophenyl)furan-2- | | | |
| | ylmethylene]-2-thioxo-imidazolidin-4- | | | |
| | one | | | |
| 95 | 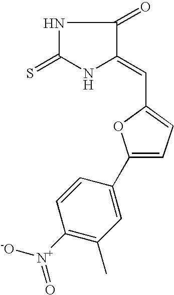 | 329.33 | * | * |
| | 5-[5-(3-Methyl-4-nitrophenyl)furan-2- | | | |
| | ylmethylene]-2-thioxo-imidazolidin-4- | | | |
| | one | | | |
| 96 | 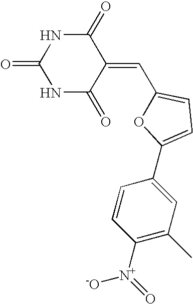 | 341.28 | * | * |
| | 5-[5-(3-Methyl-4-nitrophenyl)furan-2- | | | |
| | ylmethylene]-pyrimidine-2,4,6-trione | | | |
| 97 | 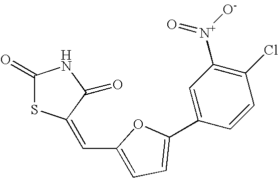 | 350.74 | * | * |
| | 5-[5-(4-Chloro-3-nitrophenyl)furan-2- | | | |
| | ylmethylene]-thiazolidine-2,4-dione | | | |
| 98 | 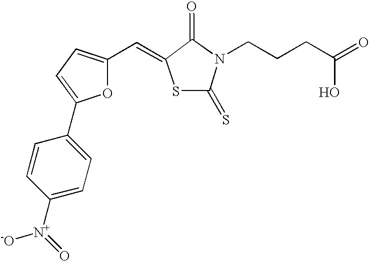 | 418.45 | * | * |
| | 4-{5-[5-(4-Nitrophenyl)furan-2- | | | |
| | ylmethylene]-4-oxo-2-thioxo-thiazolidin- | | | |
| | 3-yl}-butyric acid | | | |
| 99 | 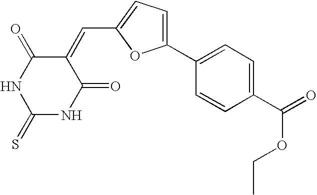 | 370.39 | * | * |
| | 4-[5-(4,6-Dioxo-2-thioxo-tetrahydro- | | | |
| | pyrimidin-5-ylidenemethyl)furan-2- | | | |
| | yl]benzoic acid ethyl ester | | | |
| 100 | 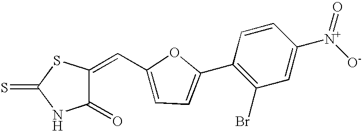 | 411.25 | * | * |
| | 5-[5-(2-Bromo-4-nitrophenyl)furan-2- | | | |
| | ylmethylene]-2-thioxo-thiazolidin-4-one | | | |
| 101 | 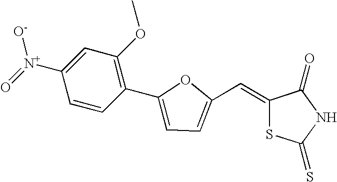 | 362.38 | * | * |
| | 5-[5-(2-Methoxy-4-nitrophenyl)furan-2- | | | |
| | ylmethylene]-2-thioxo-thiazolidin-4-one | | | |
| 102 | 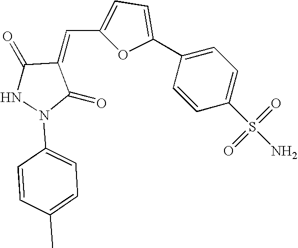 | 423.45 | * | * |
| | 4-[5-(3,5-Dioxo-1-p-tolyl-pyrazolidin-4- | | | |
| | ylidenemethyl)furan-2- | | | |
| | yl]benzenesulfonamide | | | |
| 103 | 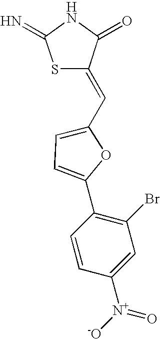 | 394.205 | * | * |
| | 5-[5-(2-Bromo-4-nitrophenyl)furan-2- | | | |
| | ylmethylene]-2-imino-thiazolidin-4-one | | | |
| 104 | 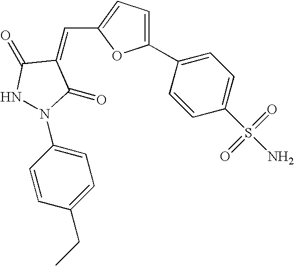 | 437.48 | * | * |
| | 4-{5-[1-(4-Ethylphenyl)-3,5-dioxo- | | | |
| | pyrazolidin-4-ylidenemethyl]furan-2- | | | |
| | yl}benzenesulfonamide | | | |
| 105 | 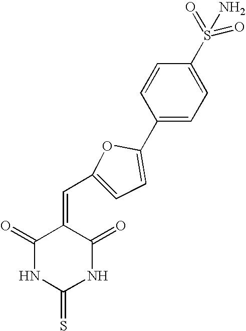 | 377.40 | * | * |
| | 4-[5-(4,6-Dioxo-2-thioxo-tetrahydro- | | | |
| | pyrimidin-5-ylidenemethyl)furan-2- | | | |
| | yl]benzenesulfonamide | | | |
| 106 | 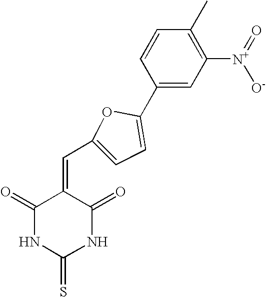 | 357.35 | * | * |
| | 5-[5-(4-Methyl-3-nitrophenyl)furan-2- | | | |
| | ylmethylene]-2-thioxo-dihydro- | | | |
| | pyrimidine-4,6-dione | | | |
| 107 | 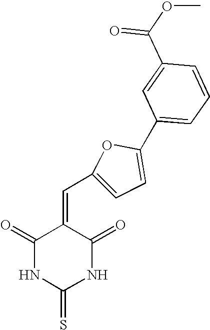 | 356.36 | * | * |
| | 3-[5-(4,6-Dioxo-2-thioxo-tetrahydro- | | | |
| | pyrimidin-5-ylidenemethyl)furan-2- | | | |
| | yl]benzoic acid methyl ester | | | |
| 108 | 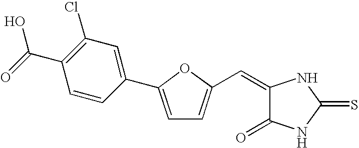 | 348.77 | * | * |
| | 2-Chloro-4-[5-(5-oxo-2-thioxo- | | | |
| | imidazolidin-4-ylidenemethyl)furan-2- | | | |
| | yl]benzoic acid | | | |
| 109 | 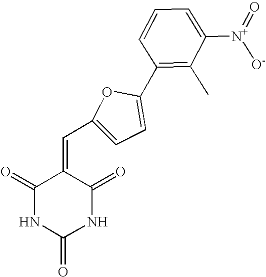 | 341.28 | * | * |
| | 5-[5-(2-Methyl-3-nitrophenyl)furan-2- | | | |
| | ylmethylene]-pyrimidine-2,4,6-trione | | | |
| 110 | 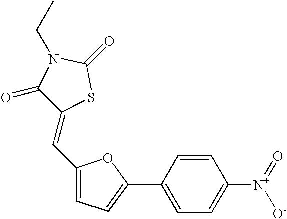 | 344.35 | * | * |
| | 3-Ethyl-5-[5-(4-nitrophenyl)furan-2- | | | |
| | ylmethylene]-thiazolidine-2,4-dione | | | |
| 111 | 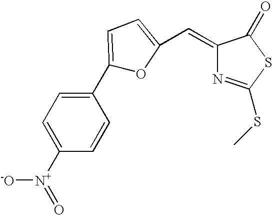 | 346.39 | * | * |
| | 2-Methylsulfanyl-4-[5-(4- | | | |
| | nitrophenyl)furan-2-ylmethylene]-4H- | | | |
| | thiazol-5-one | | | |
| 112 | 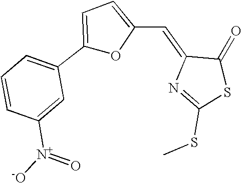 | 346.39 | * | * |
| | 2-Methylsulfanyl-4-[5-(3- | | | |
| | nitrophenyl)furan-2-ylmethylene]-4H- | | | |
| | thiazol-5-one | | | |
| 113 | 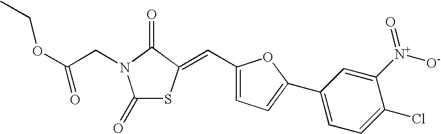 | 436.83 | * | * |
| | {5-[5-(4-Chloro-3-nitrophenyl)furan-2- | | | |
| | ylmethylene]-2,4-dioxo-thiazolidin-3-yl}- | | | |
| | acetic acid ethyl ester | | | |
| 114 | 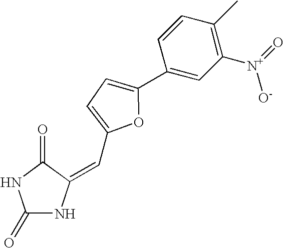 | 313.27 | * | * |
| | 5-[5-(4-Methyl-3-nitrophenyl)furan-2- | | | |
| | ylmethylene]-imidazolidine-2,4-dione | | | |
| 115 | 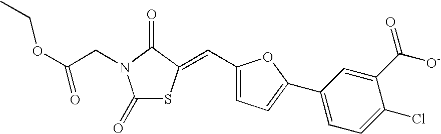 | 435.843 | * | * |
| | 2-Chloro-5-[5-(3-ethoxycarbonylmethyl- | | | |
| | 2,4-dioxo-thiazolidin-5- | | | |
| | ylidenemethyl)furan-2-yl]benzoic acid | | | |
| 116 | 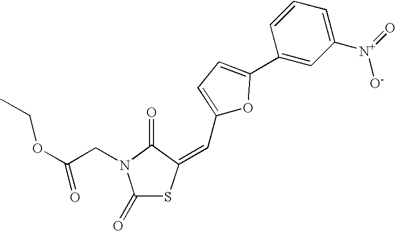 | 402.39 | * | * |
| | {5-[5-(3-Nitrophenyl)furan-2- | | | |
| | ylmethylene]-2,4-dioxo-thiazolidin-3-yl}- | | | |
| | acetic acid ethyl ester | | | |
| 117 | 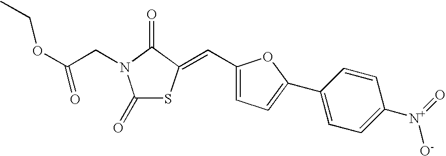 | 402.39 | * | * |
| | {5-[5-(4-Nitrophenyl)furan-2- | | | |
| | ylmethylene]-2,4-dioxo-thiazolidin-3-yl}- | | | |
| | acetic acid ethyl ester | | | |
| 118 | 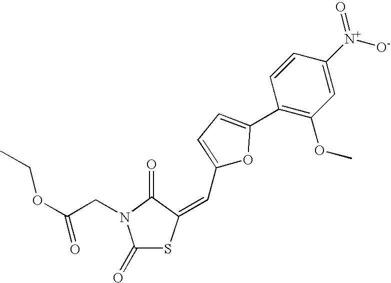 | 432.41 | * | * |
| | {5-[5-(2-Methoxy-4-nitrophenyl)furan-2- | | | |
| | ylmethylene]-2,4-dioxo-thiazolidin-3-yl}- | | | |
| | acetic acid ethyl ester | | | |
| 119 | 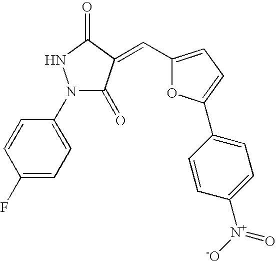 | 393.33 | * | * |
| | 1-(4-Fluorophenyl)-4-[5-(4- | | | |
| | nitrophenyl)furan-2-ylmethylene]- | | | |
| | pyrazolidine-3,5-dione | | | |
| 120 | 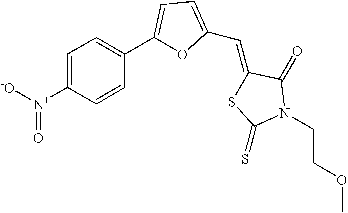 | 390.44 | * | * |
| | 3-(2-Methoxy-ethyl)-5-[5-(4- | | | |
| | nitrophenyl)furan-2-ylmethylene]-2- | | | |
| | thioxo-thiazolidin-4-one | | | |
| 121 | 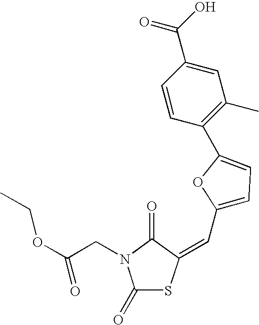 | 415.42 | * | * |
| | 4-[5-(3-Ethoxycarbonylmethyl-2,4-dioxo- | | | |
| | thiazolidin-5-ylidenemethyl)furan-2-yl]- | | | |
| | 3-methylbenzoic acid | | | |
| 122 | 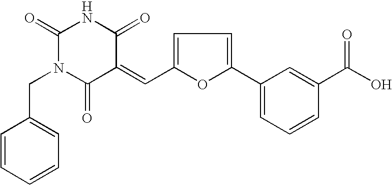 | 416.39 | * | * |
| | 3-[5-(1-benzyl-2,4,6-trioxo-tetrahydro- | | | |
| | pyrimidin-5-ylidenemethyl)furan-2- | | | |
| | yl]benzoic acid | | | |
| 123 | 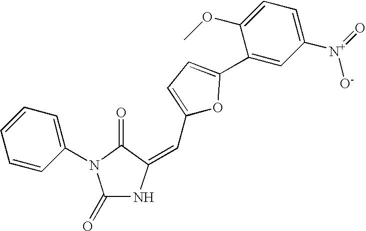 | 405.37 | * | * |
| | 5-[5-(2-Methoxy-5-nitrophenyl)furan-2- | | | |
| | ylmethylene]-3-phenyl-imidazolidine-2,4- | | | |
| | dione | | | |
| 124 | 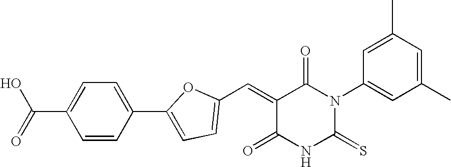 | 446.49 | * | * |
| | 4-{5-[1-(3,5-Dimethylphenyl)-4,6-dioxo- | | | |
| | 2-thioxo-tetrahydro-pyrimidin-5- | | | |
| | ylidenemethyl]furan-2-yl}benzoic acid | | | |
| 125 | 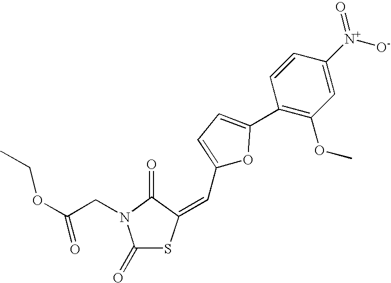 | 418.38 | * | * |
| | {5-[5-(2-Methoxy-4-nitrophenyl)furan-2- | | | |
| | ylmethylene]-2,4-dioxo-thiazolidin-3-yl}- | | | |
| | acetic acid methyl ester | | | |
| 126 | 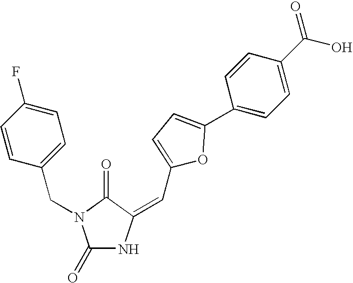 | 406.37 | * | * |
| | 4-{5-[1-(4-Fluorobenzyl)-2,5-dioxo- | | | |
| | imidazolidin-4-ylidenemethyl]furan-2- | | | |
| | yl}benzoic acid | | | |
| 127 | 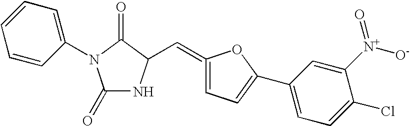 | 409.79 | * | * |
| | 5-[5-(4-Chloro-3-nitrophenyl)furan-2- | | | |
| | ylmethylene]-3-phenyl-imidazolidine-2,4- | | | |
| | dione | | | |
| 128 | 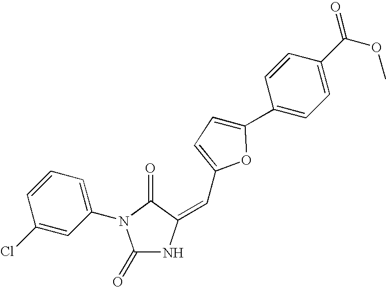 | 422.83 | * | * |
| | 4-{5-[1-(3-Chlorophenyl)-2,5-dioxo- | | | |
| | imidazolidin-4-ylidenemethyl]furan-2- | | | |
| | yl}benzoic acid methyl ester | | | |
| 129 | 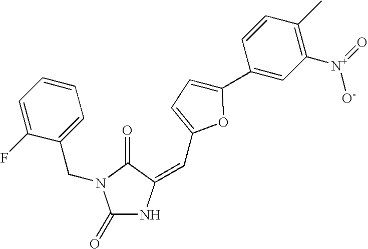 | 421.39 | * | * |
| | 3-(2-Fluorobenzyl)-5-[5-(4-methyl-3- | | | |
| | nitrophenyl)furan-2-ylmethylene]- | | | |
| | imidazolidine-2,4-dione | | | |
| 130 | 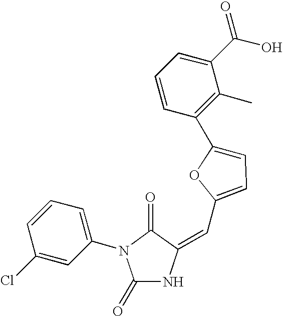 | 422.83 | * | * |
| | 3-{5-[1-(3-Chlorophenyl)-2,5-dioxo- | | | |
| | imidazolidin-4-ylidenemethyl]furan-2- | | | |
| | yl}-2-methylbenzoic acid | | | |
| 131 | 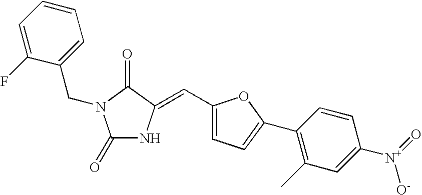 | 421.39 | * | * |
| | 3-(2-Fluorobenzyl)-5-[5-(2-methyl-4- | | | |
| | nitrophenyl)furan-2-ylmethylene]- | | | |
| | imidazolidine-2,4-dione | | | |
| 132 | 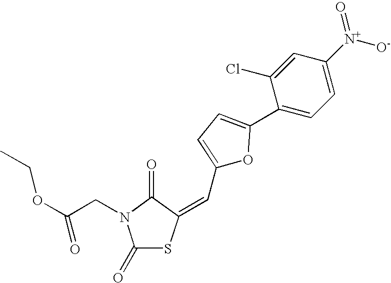 | 436.83 | * | * |
| | {5-[5-(2-Chloro-4-nitrophenyl)furan-2- | | | |
| | ylmethylene]-2,4-dioxo-thiazolidin-3-yl}- | | | |
| | acetic acid ethyl ester | | | |
| 133 | 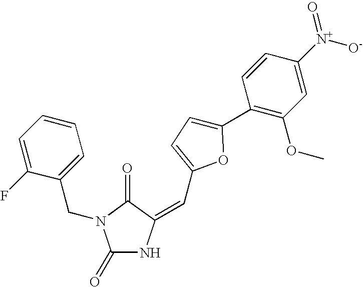 | 437.39 | * | * |
| | 3-(2-Fluorobenzyl)-5-[5-(2-methoxy-4- | | | |
| | nitrophenyl)furan-2-ylmethylene]- | | | |
| | imidazolidine-2,4-dione | | | |
| 134 | 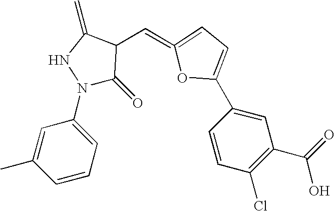 | 422.83 | * | * |
| | 2-Chloro-5-[5-(3,5-dioxo-1-m-tolyl- | | | |
| | pyrazolidin-4-ylidenemethyl)furan-2- | | | |
| | yl]benzoic acid | | | |
| 135 | 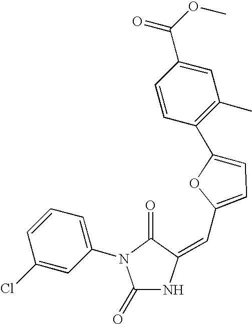 | 436.86 | * | * |
| | 4-{5-[1-(3-Chlorophenyl)-2,5-dioxo- | | | |
| | imidazolidin-4-ylidenemethyl]furan-2- | | | |
| | yl}-3-methylbenzoic acid methyl ester | | | |
| 136 | 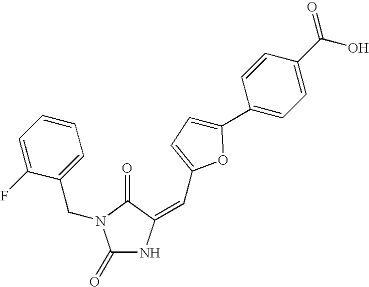 | 406.37 | * | * |
| | 4-{5-[1-(2-Fluorobenzyl)-2,5-dioxo- | | | |
| | imidazolidin-4-ylidenemethyl]furan-2- | | | |
| | yl}benzoic acid | | | |
| 137 | 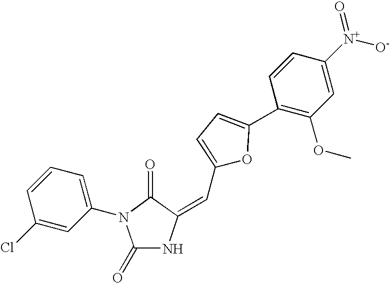 | 439.82 | * | * |
| | 3-(3-Chlorophenyl)-5-[5-(2-methoxy-4- | | | |
| | nitrophenyl)furan-2-ylmethylene]- | | | |
| | imidazolidine-2,4-dione | | | |
| 138 | 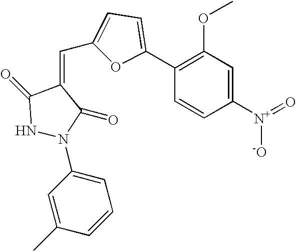 | 419.40 | * | * |
| | 4-[5-(2-Methoxy-4-nitrophenyl)furan-2- | | | |
| | ylmethylene]-1-m-tolyl-pyrazolidine-3,5- | | | |
| | dione | | | |
| 139 | 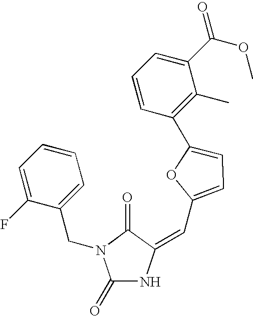 | 434.43 | * | * |
| | 3-{5-[1-(2-Fluorobenzyl)-2,5-dioxo- | | | |
| | imidazolidin-4-ylidenemethyl]furan-2- | | | |
| | yl}-2-methylbenzoic acid methyl ester | | | |
| 140 | 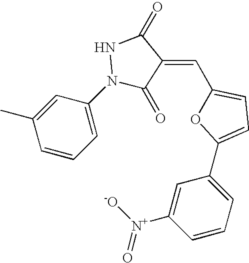 | 389.37 | * | * |
| | 4-[5-(3-Nitrophenyl)furan-2- | | | |
| | ylmethylene]-1-m-tolyl-pyrazolidine-3,5- | | | |
| | dione | | | |
| 141 | 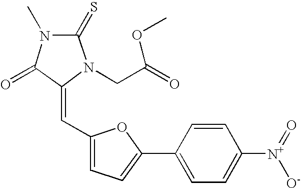 | 401.40 | * | * |
| | {3-Methyl-5-[5-(4-nitrophenyl)furan-2- | | | |
| | ylmethylene]-4-oxo-2-thioxo- | | | |
| | imidazolidin-1-yl}-acetic acid methyl | | | |
| | ester | | | |
| 142 | 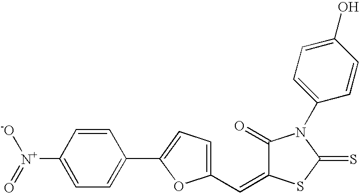 | 424.46 | * | * |
| | 3-(4-Hydroxyphenyl)-5-[5-(4- | | | |
| | nitrophenyl)furan-2-ylmethylene]-2- | | | |
| | thioxo-thiazolidin-4-one | | | |
| 143 | 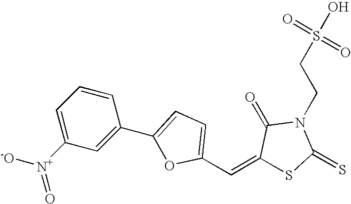 | 440.48 | * | * |
| | 2-{5-[5-(3-Nitrophenyl)furan-2- | | | |
| | ylmethylene]-4-oxo-2-thioxo-thiazolidin- | | | |
| | 3-yl}-ethanesulfonic acid | | | |

% MAXIMAL BINDING KEY
0 < XXXXX < 10
10 < XXXX < 20
20 < XXX < 30
30 < XX < 40
40 < X < 50
50 < *

In another embodiment, the invention is directed to a compound of formula (6): 4-[5-(3-Carboxymethyl-4-oxo-2-thioxo-thiazolidin-5-ylidenemethyl)furan-2-yl]benzoic acid ethyl ester:

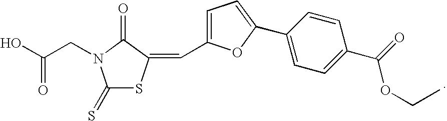

In another embodiment, the invention is directed to a compound of formula (5): 3-[5-(1-Methyl-2,4,6-trioxo-tetrahydro-pyrimidin-5-ylidenemethyl)furan-2-y]benzoic acid:

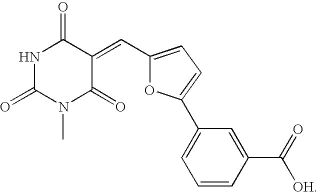

In yet another embodiment, the invention is directed to a compound of formula (5): 2-Chloro-4-[5-(4,6-dioxo-2-thioxo-tetrahydro-pyrimidin-5-ylidenemethyl)furan-2-yl]benzoic acid:

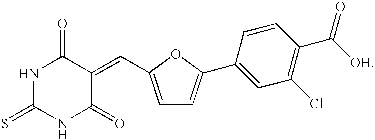

In yet another embodiment, the invention is directed to another compound of formula (5): 4-{5-[1-(2-Fluorophenyl)-2,4,6-trioxo-tetrahydro-pyrimidin-5-ylidenemethyl]furan-2-yl}-3-methylbenzoic acid:

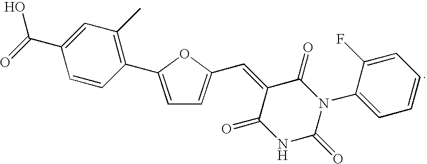

In still another embodiment, the invention is directed to another compound of formula (6): 2-Chloro-4-[5-(3-methoxycarbonylmethyl-2,4-dioxo-thiazolidin-5-ylidenemethyl)furan-2-yl]benzoic acid:

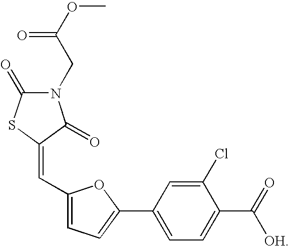

In still another embodiment, the invention is directed to another compound of formula (6): 3-[5-(3-Carboxymethyl-4-oxo-2-thioxo-thiazolidin-5-ylidenemethyl)furan-2-yl]benzoic acid methyl ester:

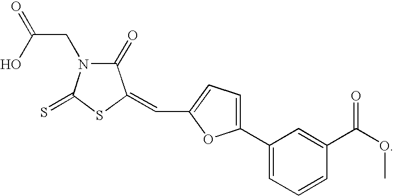

In a preferred embodiment, the NGF/NTR or NGF/BDNFINTR modulator exhibits greater modulation in cells which express p75^{NTR} but not TrkA than in cells which express both p75^{NTR} and TrkA. The interaction of NGF with p75^{NTR} in cells which do not express TrkA can, under certain conditions, mediate apoptotic cell death. The p75^{NTR} receptor has a greater affinity for NGF in this proapoptotic state, that is, in cells which do not express TrkA. Compounds which exhibit greater NGF/NTR or NGF/BDNF/NTR modulation in the absence of TrkA advantageously selectively modulate or interfere with processes such as apoptotic cell death, while having a smaller effect on other p75^{NTR}-mediated processes.

In one embodiment of the invention, the modulating compounds of the invention are capable of chemically interacting with NGF, BDNF, p75^{NTR}, TrkA and/or TrkB. The language “chemical interaction” is intended to include, but is not limited to reversible interactions such as hydrophobic/hydrophilic, ionic (e.g., coulombic attraction/repulsion, ion-dipole, charge-transfer), covalent bonding, Van der Waals, and hydrogen bonding. In certain embodiments, the chemical interaction is a reversible Michael addition. In a specific embodiment, the Michael addition involves, at least in part, the formation of a covalent bond.

Compounds of the inventions can be synthesized according to standard organic synthesis procedures that are known in the art.

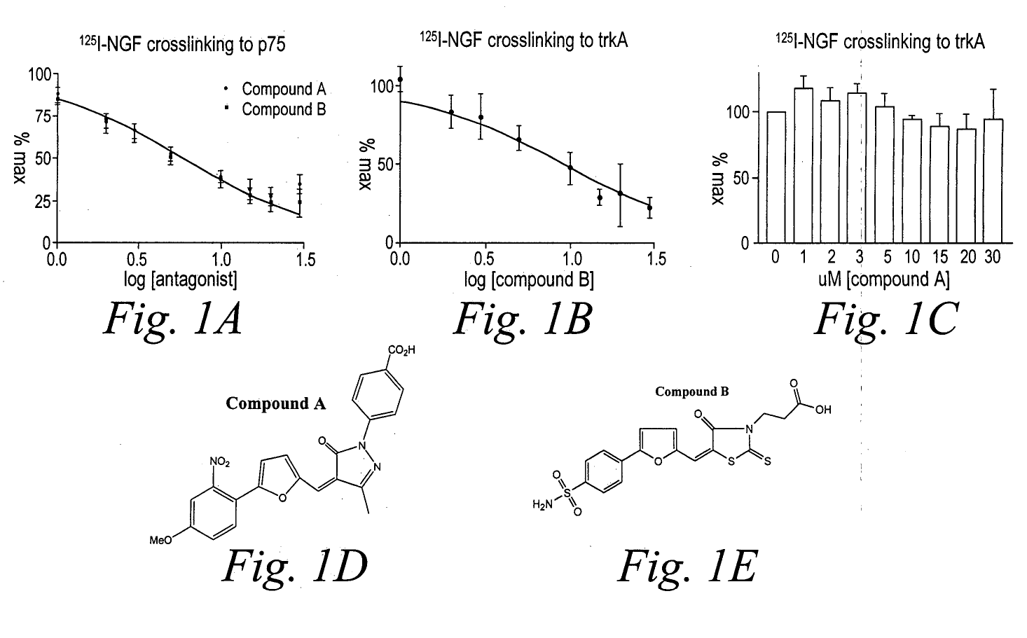

Below is a scheme for compound A from FIG. 1D using organic starting materials and synthetic procedures well-known in organic chemistry synthesis:

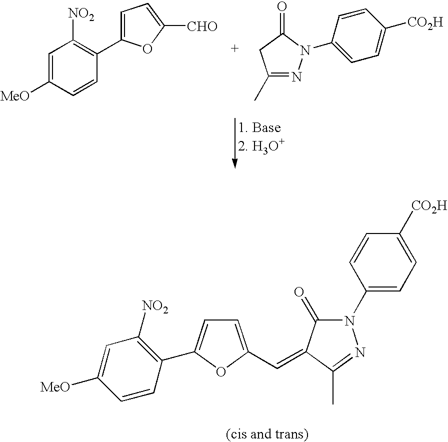

Below is a scheme for compound B from FIG. 1E using organic starting materials and synthetic procedures well-known in organic chemistry synthesis:

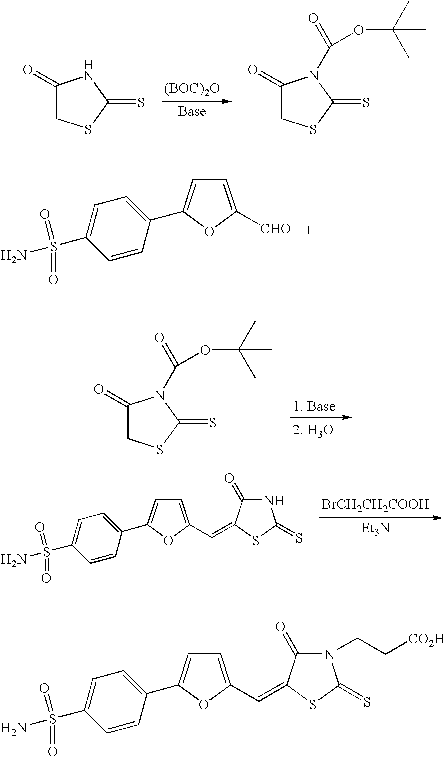

Acid addition salts of the compounds of Formulas 1, 2, 3, 3A, 4, 5 and 6, and the compounds of Table 1, Table 2, Table 3 and Table 4 are most suitably formed from pharmaceutically acceptable acids, and include for example those formed with inorganic acids e.g. hydrochloric, sulphuric or phosphoric acids and organic acids e.g. succinic, maleic, acetic or fumaric acid. Other non-pharmaceutically acceptable salts e.g. oxalates may be used for example in the isolation of the compounds of Formulas 1, 2, 3, 3A, 4, 5 and 6, and the compounds of Table 1, Table 2, Table 3 and Table 4 for laboratory use, or for subsequent conversion to a pharmaceutically acceptable acid addition salt. Also included within the scope of the invention are solvates and hydrates of the invention.

The conversion of a given compound salt to a desired compound salt is achieved by applying standard techniques, in which an aqueous solution of the given salt is treated with a solution of base e.g. sodium carbonate or potassium hydroxide, to liberate the free base which is then extracted into an appropriate solvent, such as ether. The free base is then separated from the aqueous portion, dried, and treated with the requisite acid to give the desired salt.

In vivo hydrolyzable esters or amides of certain compounds of Formulas 1, 2, 3, 3A, 4, 5 and 6, and the compounds of Table 1, Table 2, Table 3 and Table 4 can be formed by treating those compounds having a free hydroxy or amino functionality with the acid chloride of the desired ester in the presence of a base in an inert solvent such as methylene chloride or chloroform. Suitable bases include triethylamine or pyridine. Conversely, compounds of Formulas 1, 2, 3, 3A, 4, 5 and 6, and the compounds of Table 1, Table 2, Table 3 and Table 4 having a free carboxy group may be esterified using standard conditions which may include activation followed by treatment with the desired alcohol in the presence of a suitable base.

Assays

The present invention also relates to a method of modulating NT/NTR, NGF/NTR, BDNF/NTR or NGF/BDNF/NTR interactions. In certain embodiments, the method comprises contacting NGF and/or BDNF and/or precursors thereof in the presence of p75^{NTR} with an NGF/BDNF/NTR modulating amount of a NGF/BDNF/NTR modulator compound, thereby modulating the interaction of NGF and/or BDNF and/or precursors thereof with p75^{NTR} and/or TrkA and/or TrkB.

The present invention also relates to a method of modulating the interaction of BDNF (or proBDNF) with a neurotrophin receptor, e.g., p75^{NTR}. The method comprises contacting BDNF in the presence of p75^{NTR} and/or TrkA and/or TrkB with a BDNF/NTR modulating amount of a BDNF/NTR modulating compound, thereby modulating the interaction of BDNF with p75^{NTR}. The present invention also relates to a method of modulating the interaction of NGF (or proNGF) with a neurotrophin receptor, e.g., p75^{NTR}. The method comprises contacting NGF (or proNGF) in the presence of p75^{NTR} and/or TrkA and/or TrkB with a NGF/NTR modulating amount of a NGF/NTR modulating compound, thereby modulating the interaction of NGF with p75^{NTR}.

The methods of the invention can be practiced in vitro, for example, in a cell culture screening assay to screen compounds which potentially bind, activate or modulate receptor function. In such a method, the modulating compound can function by interacting with and eliminating any competing function (e.g., receptor binding) of BDNF and/or NGF and/or a precursor thereof in the sample or culture. The modulating compounds can also be used to control BDNF and or NGF activity in neuronal cell culture. In such a method, the compound can function by interacting with and eliminating any competing function (e.g., binding) of NGF and BDNF in the sample or culture. The modulating compounds can also be used to control NGF and BDNF activity in neuronal cell culture. In vitro cross-linking assays for determining the ability of a compound within the scope of the invention to modulate the interaction of NGF with p75^{NTR} and/or TrkA, and/or to modulate the interaction of BDNF with p75^{NTR} and/or TrkB are well known in the art and described in Example 1. Other assays for determining the ability of a compound to modulate the activity of NGF and BDNF with their respective receptors are also readily available to the skilled artisan (see, Barker et al., Neuron 13(1): 203-215; (1994), Dehant et al., Development 119: 545-25 558 (1993); and US 2002/016982).

Recombinant and native neurotrophin polypeptides from different species, including humans, are commercially available from several sources (e.g., Promega Corporation and Regeneron Pharmaceuticals). In addition neurotrophin polypeptides for use in the assays described herein can be readily produced by standard biological techniques or by chemical synthesis. For example, a host cell transfected with an expression vector containing a nucleotide sequence encoding the desired neurotrophin can be cultured under appropriate conditions to allow expression of the peptide to occur. The secreted peptide can then be isolated according to standard techniques. Coding polynucleotides, precursors and promoters for a number of neurotrophins are known, including coding sequences for neurotrophins of some mammalian species. For example, GenBank M61176 sets for the coding sequence for BDNF (see also, XM.006027); BDNF precursor is set forth at BF439589; and a BDNF specific promoter is set forth at Eo5933. A similar range of coding sequences for other neurotrophins, including pro-NGF and mature NGF (e.g., NCBI ACCESSION NO P01138 and CAA37703), NT-4/5 and NT-3, are also available through GenBank and other publicly accessible nucleotide and amino acid sequence databases. Alternatively, the neurotrophin, e.g., BDNF or NGF can be obtained by culturing a primary cell culture or an established cell line that can produce the neurotrophin, and isolating from the culture broth thereof (e.g., culture supernatant, cultured cells).

The method can also be practiced in vivo, for example, to modulate one or more processes mediated by the interaction of NGF (and/or proNGF) to p75^{NTR}, the interaction of BDNF (and/or proBDNF) to p75^{NTR}, or both and/or the interaction of NGF and/or BDNF and/or a precursor thereof to TrkA and TrkB, respectively. Animal models for determining the ability of a compound of the invention to modulate a neurotrophin-mediated biological activity are well known and readily available to the skilled artisan. Useful animal models of neurotrophin-mediated biologicial activity are well known in the art. For example, animal models of neuropathic pain are described in Zeltser et al., 2000, Pain 89:19-24; Bennett et al., 1988, Pain 33:87-107; Seltzer et al., 1990, Pain 43:205-218; Kim et al., 1992, Pain 20 50:355-363; and Decosterd et al., 2000, Pain 87:149-158. An animal model of inflammatory pain using complete Freund's adjuvant is described in Jasmin et al., 1998, Pain 75: 367-382. A stress-induced hyperalgesia model is described in Quintero et al., 2000, Pharmacology, Biochemistry and Behavior 67:449-458. Further animal models for pain are considered in an article of Walker et al. 1999 Molecular Medicine Today 5:319-321, comparing models for different types of pain, which are acute pain, chronic/inflammatory pain and chronic/neuropathic pain, on the basis of behavioral signs. Animal models for depression are described by E. Tatarczynska et al., Br. J. Pharmacol. 132(7): 1423-1430 (2001) and P. J. M. Will et al., Trends in Pharmacological Sciences 22(7):331-37 (2001)); models for anxiety are described by D. Treit, “Animal Models for the Study of Anti-anxiety Agents: A Review,” Neuroscience & Biobehavioral Reviews 9(2):203-222 (1985).

Accordingly, an agent identified as described herein (e.g., a NT/NTR, NGF/NTR, NGF/BDNF/NTR modulator or a BDNF/NTR modulator) can be used in an animal model to determine the efficacy, toxicity, or side effects of treatment with such an agent. Alternatively, an agent identified as described herein can be used in an animal model to determine the mechanism of action of such an agent.

Accordingly, this invention pertains to uses of novel agents identified by the above-described screening assays for treatments as described herein

Pharmaceutical Compositions

The present invention also provides pharmaceutical compositions. Such compositions comprise a therapeutically (or prophylactically) effective amount of one or more NT/NTR, NGF/NTR, NGF/BDNF/NTR and BDNF/NTR modulators, preferably one or more compounds of Formulas 1, 2, 3, 3A, 4, 5 and 6, and the compounds of Table 1, Table 2, Table 3 and Table 4 described above, and a pharmaceutically acceptable carrier or excipient. Suitable pharmaceutically acceptable carriers include, but are not limited to, saline, buffered saline, dextrose, water, glycerol, ethanol, and combinations thereof. The carrier and composition can be sterile. The formulation should suit the mode of administration.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the subject. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelating agents, such as citric acid, ethylenediamine tetraacetic acid (EDTA), sorbitol, tartaric acid, phosphoric acid, and the like.

Suitable pharmaceutically acceptable carriers include but are not limited to water, salt solutions (e.g., NaCl), alcohols, gum arabic, vegetable oils, benzyl alcohols, polyethylene glycols, gelatin, carbohydrates such as lactose, amylose or starch, cyclodextrin, magnesium stearate, talc, silicic acid, viscous paraffin, perfume oil, fatty acid esters, hydroxymethylcellulose, polyvinyl pyrolidone, etc. The pharmaceutical preparations can be sterilized and if desired, mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active compounds.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as trilycerides. Oral formulation can include standard carriers such as pharmaceutical grades of mannitol, lactose, starch, magnesium stearate, polyvinyl pyrollidone, sodium saccharine, cellulose, magnesium carbonate, etc.

The composition can be formulated in accordance with the routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachet indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

The pharmaceutical compositions of the invention can also include an agent which controls release of the NT/NTR, NGF/NTR, NGF/BDNF/NTR and BDNF/NTR modulator compounds, thereby providing a timed or sustained relase composition.

The present invention also relates to prodrugs of the NT/NTR, NGF/NTR, NGF/BDNF/NTR and BDNF/NTR modulators disclosed herein, as well as pharmaceutical compositions comprising such prodrugs. For example, compounds of the invention which include acid functional groups or hydroxyl groups can also be prepared and administered as a corresponding ester with a suitable alcohol or acid. The ester can then be cleaved by endogenous enzymes within the subject to produce the active agent.

Formulations of the present invention include those suitable for oral, nasal, topical, transdermal, buccal, sublingual, rectal, vaginal and/or parenteral administration. The formulations may conveniently be presented in unit dosage form and may be prepared by any methods well known in the art of pharmacy. The amount of active ingredient that can be combined with a carrier material to produce a single dosage form will generally be that amount of the compound that produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluent commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

Methods of Administration

The pharmaceutical compositions containing the compounds of the invention may be given orally, parenterally, topically, or rectally. They are of course given by forms suitable for each administration route. For example, they are administered in tablets or capsule form, by injection, inhalation, eye lotion, ointment, suppository, etc. administration by injection, infusion or inhalation; topically by lotion or ointment; and rectally by suppositories. Oral administration is preferred.

The phrases “parenteral administration” and “administered parenterally” as used herein mean modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the subject's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular subject, composition, and mode of administration, without being toxic to the subject.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the subject being treated, and like factors well known in the medical arts.

A physician or veterinarian having ordinary skill in the art can readily determine and prescribe the effective amount of the pharmaceutical composition required. For example, dosages of a compound of the invention may be determined by deriving dose-response curves using an animal model for the condition to be treated. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound that is the lowest dose effective to produce a therapeutic effect. Such an effective dose will generally depend upon the factors described above. Generally, intravenous and subcutaneous doses of the compounds of this invention for a subject, when used for the indicated analgesic effects, will range from about 0.0001 to about 100 mg per kilogram of body weight per day, more preferably from about 0.01 to about 100 mg per kg per day, and still more preferably from about 1.0 to about 50 mg per kg per day. An effective amount is that amount treats a neurotrophin-associated state or neurotrophin disorder.

If desired, the effective daily dose of the active compound may be administered as two, three, four, five, six or more sub-doses administered separately at appropriate intervals throughout the day, optionally, in unit dosage forms.

While it is possible for a compound of the present invention to be administered alone, it is preferable to administer the compound as a pharmaceutical composition.

Methods of Treatment

The above compounds can be used for administration to a subject for the modulation of a neurotrophin-mediated activity, including, but not limited to pain, inflammatory and neurological disorders, including disorders and states (e.g., a disease state) that are associated with neurotrophin-mediated activity, including, but not limited to, abnormal neuron growth, abnormal neuron proliferation or abnormal neuron function, abnormal neurotransmission and/or any abnormal function of cells, organs, or physiological systems that are modulated, at least in part, by a neurotrophin-mediated activity.

Accordingly, the compounds of the invention may be used to treat pain, and it is understood that the compounds may also alleviate or treat one or more additional symptoms of a disease or disorder discussed herein, e.g., inflammatory and/or neurological disorder.

Examples of pain that may be treated according to the methods of the invention include, but are not limited to pain associated with injury, trauma, cutaneous pain, somatic pain, visceral pain, neuropathic pain, acute pain, chronic malignant pain, chronic nonmalignant pain, post-operative pain, cancer pain and inflammatory pain.

Examples of cutaneous pain include, but are not limited to pain related to cuts, burns, lacerations, punctures, incisions, surgical pain, post-operative pain, including orodental surgery, and pain associated with inflammation and infection.

Examples of somatic pain include, but are not limited to arthralgia, myalgia, myofascial pain syndrome, chronic lower back pain, cancer-associated pain, phantom limb pain, central pain, bone injury pain, dental pain, fibromyalgia syndrome, meralgia paraesthetica, fibrocitis, idiopathic pain disorder, atypical odontalgia, loin pain, non-cardiac chest pain, chronic nonspecific pain, musculoskeletal pain disorder, chronic pelvic pain, and pain during labor and delivery, post-operative pain, cluster headaches, surgical pain, pain resulting from severe, for example third degree, burns, post partum pain, postmastectomy pain syndrome, stump pain, referred pain, reflex sympathetic dystrophy, and causalgia.

Examples of somatic pain further include, but are not limited to, pain related to injuries, diseases or disorders associated with the musculoskeletal system and connective tissues. Examples of musculoskeletal system and connective tissue injuries and disorders include, sprains, broken bones, arthropathies (e.g., various forms of arthritis, rheumatoid arthritis, osteoarthritis and gout), dorsopathies (e.g., various forms of scoliosis, kyphosis, lordosis, osteochondrosis, spondylolysis, subluxation, and torticollis), myositis and diseases of the muscles (e.g., infective myositis, interstitial myositis, calcification and ossification of muscle, diastasis of muscle, ischaemic infarction of muscle, and muscle strain), osteopathies and chondropathies (e.g., various forms of osteoporosis with or without pathological fracture including, but not limited to postmenopausal, drug-induced, and idiopathic osteoporosis.), osteomalacia, disorders of continuity of bone (e.g., malunion or nonunion of fracture, stress fracture, and pathological fracture), disorders of bone density and structure (e.g., fibrous dysplasia, skeletal fluorosis, osteitis and condensans) and disorders of the skin (e.g., psoriasis, eczema and dermatitis).

Additional examples of somatic pain include, but are not limited to, pain related to injuries, diseases or disorders associated with the circulatory system. Examples of circulatory system injuries and disorders include, but are not limited to, acute and chronic rheumatic heart diseases; hypertensive diseases; ischaemic heart diseases. including angina pectoris, acute myocardial infarction, coronary thrombosis, coronary insufficiency, mitral insufficiency, hypertrophic cardiomyopathy, ventricular cardiac arrhythmia (e.g., sustained ventricular tachydardia, non-sustained ventricular tachycardia, ventricular fibrillation, ventricular premature beats and ventricular flutter), and atrial tachyarrhythmia (e.g., atrial fibrillation and atrial flutter) and Dressler's syndrome; diseases of arteries, arterioles and capillaries (e.g., atherosclerosis, aneurysm, peripheral vascular disease, Raynaud's syndrome, Buerger, intermittent claudication, acrocyanosis and erythrocyanosis); diseases of veins, lymphatic vessels and lymph nodes (e.g., various forms of phlebitis, thrombophlebitis, embolism or thrombosis of the veins, varicose veins, haemorrhoids, varices, (including oesophageal, gastric, scrotal, pelvic), lymphadenitis, lymphangitis, and lymphoedema.

Disorders of the respiratory system associated with visceral pain include, but are not limited to, acute and chronic upper respiratory infections (e.g., various forms or manifestations of acute or chronic nasopharyngitis, sinusitis, pharyngitis, tonsillitis, laryngitis, tracheitis, laryngotracheitis, rhinitis), influenza, various forms of pneumoniae (e.g., bacterial, viral, parasitic or fungal), acute or chronic lower respiratory infections (e.g., various forms or manifestations of acute or chronic bronchitis, bronchiolitis, tracheobronchitis, emphysema, bronchiectasis, status asthmaticus, asthma and other chronic obstructive pulmonary diseases (COPD), adult respiratory distress syndrome, pulmonary oedema, pyothorax, diseases of the pleura.

Disorders of the gastrointestinal system associated with visceral pain include, but are not limited to, disorders of the tooth (e.g., anodontia, supernumerary teeth, mottled teeth, teething syndrome, embedded and impacted teeth, dental caries, pathological resorption, ankylosis of teeth, hypercementosis, pulpitis, necrosis or degeneration of the pulp, various forms of acute or chronic gingivitis, periodontitis, or periodontal disease, gingival recession); dentofacial anomalies (e.g., manadibular hyper- or hypoplasia, asymmetry of jaw, retrognathism, crossbite, temporomandibular joint disorders), orodental cysts, inflammatory conditions of jaws, diseases of the salivary glands (e.g., sialoadenitis, sialolithiasis, abscess, fistula or mucocele of the salivary gland); diseases of the lip and oral mucosa (e.g., various forms of stomatitis, recurrent oral aphthae, cellulites and abscess of the mouth); diseases of the tongue (e.g., various forms of glossitis, glossodynia, hypertrophy of tongue papillae); diseases of the oesophagus, stomach and duodenum (e.g., oesophagitis, Gastro-oesophageal reflux disease, Achalasia of cardia, ulcer of oesophagus, dyskinesia of oesophagus, diverticulum of oesophagus, ulcers (e.g., oesophageal, gastric, duodenal, gastrojejunal); various forms and manifestations of gastritis and duodenitis, (e.g., dyspepsia, pyloric stenosis, pylorospasms), esophageal varicies and refractory ascites and esophageal carcinoma; diseases of the appendix (e.g., various forms and manifestations of appendicitis); hernia (e.g,. various forms and manifestations of inguinal, femoral, umbilical, ventral, or diaphragmatic hernia), diseases of the intestines including noninfective enteritis and colitis (e.g., various forms and manifestations of Crohn's disease, ulcerative colitis, collagenous colitis, gastroenteritis and colitis due to radiation, toxic, allergic or dietetic gastroenteritis and colitis, as well as various forms of ileitis, jejunitis or sigmoiditis), acute or chronic vascular disorders of the intestine (e.g., fulminant ischaemic colitis, intestinal infarction, chronic ischaemic colitis or enteritis, mesenteric vascular insufficiency, angiodysplasia of colon), paralytic ileus and intestinal obstruction (e.g., volvulus, gallstone ileus, intestinal occlusion), diverticular disease of intestine with or without perforation and/or abscess (e.g., diverticulitis, diverticulosis, diverticulum), irritable bowel syndrome, constipation, diarrhea (e.g., functional diarrhea, and infectious diarrhea such as diarrhea associated with amebiasis, giardiasis, viral infection, cytomegalovirus infection, or pathogenic bacterial infection), neurogenic bowel, megacolon, anal spasm (proctalgia fugax), fissure, fistula or abscess of anal and rectal regions, anal or rectal polyp, anal or rectal prolapse, anal or rectal stenosis, ulcer of anus or rectum, radiation proctitis), diseases of the peritoneum (e.g. various forms and manifestations of acute or chronic peritonitis, peritoneal adhesions, haemoperitoneum), diseases of the liver such as various forms and manifestations of alcoholic liver disease (e.g., alcoholic fatty liver, alcoholic hepatitis, alcoholic fibrosis and sclerosis of liver, alcoholic cirrhosis of liver, alcoholic hepatic failure), various forms and manifestations of acute, subacute or chronic hepatic failure, of acute and chronic hepatitis (e.g., nonspecific reactive hepatitis, autoimmune hepatitis, chronic persistent hepatitis, chronic lobular hepatitis, granulomatous hepatitis, infectious or parasitic hepatitis (e.g., cytomegaloviral, herpesviral, toxoplasma) of fibrosis and cirrhosis of liver (e.g. hepatic fibrosis and/or sclerosis, cardiac sclerosis of liver, primary or secondary biliary cirrhosis, macronodular cirrhosis, cryptogenic cirrhosis), liver necrosis, infarction of liver, hepatic veno-occlusive disease, Budd-Chiari syndrome, portal hypertension, hepatorenal syndrome, focal nodular hyperplasia of liver, hepatoptosis, various forms and manifestations of toxic or idiosyncratic liver disease; disorders of gallbladder, biliary tract and pancreas (e.g., cholelithiasis, cholecystolithiasis, choledocholithiasis, gallstone or calculus of gallbladder with or without cholecystitis), gallstone or calculus of bile duct (with or without cholecystitis and/or cholangitis), acute or chronic cholecystitis (e.g., emphysematous, gangrenous or suppurative cholecystitis, empyema or gangrene of gallbladder), occlusion, stenosis or stricture of cystic duct or gallbladder without calculus, hydrops, perforation or fistula of gallbladder, cholecystocolic or cholecystoduodenal fistula, cholesterolosis of gallbladder, cholangitis (e.g., ascending, primary, secondary, recurrent, sclerosing, or stenosing cholangitis), obstruction of the bile duct without calculus, spasm of sphincter of Oddi, biary cyst, various forms and manifestations of pancreatitis (e.g., acute, subacute or chronic pancreatitis, infectious pancraetitis, haemorrhagic pancreatitis, suppurative pancreatitis), pancreatic steatorrhoea, cyst or pseudocyst of the pancreas, coeliac disease, gluten-sensitive enteropathy, idiopathic steatorrhoea, gastric or intestinal haemorrhage, functional abdominal pain syndrome (FAPS), gastrointestinal motility disorders, faecal incontinence, and various forms of cancer and neoplasm of the gastrointestinal system (e.g., neoplasm of the oesophagus, stomach, small intestine, colon, liver and pancreas)

Disorders of the genitourinary system associated with visceral pain include, but are not limited to, glomerular diseases nephritic syndromes (e.g., glomerulonephritis, nephritis, acute, infectious or chronic tubulo-interstitial nephritis, diffuse sclerosing glomerulonephritis, recurrent and persistent haematuria), nephrotic syndrome, lipoid nephrosis, proteinuria (e.g., Bence Jones, gestational, orthostatic, persistent), glomerular disorders in other diseases (e.g., infectious and parasitic diseases, blood diseases and disorders involving the immune mechanism, diabetes mellitus, systemic connective tissue disorders, endocrine, nutritional and metabolic diseases)], acute or chronic renal tubulo-interstitial diseases (e.g., interstitial nephritis, infectious interstitial nephritis, pyelitis, pyelonephritis), chronic obstructive or non-obstructive pyelonephritis, obstructive and reflux uropathy, nephropathy (e.g., analgesic nephropathy, nephrpathy induced by drugs, medicaments, heavy metals and biological substances, renal tubulo-interstitial disorders in other diseases (e.g., in infectious and parasitic diseases, in neoplastic diseases, in blood diseases and disorders involving the immune, in metabolic diseases, in systemic connective tissue disorders, or in transplant rejection), acute or chronic renal failure (with tubular, cortical or medullary necrosis) uraemia, urolithiasis (e.g., calculus of kidney and/or ureter, nephrolithiasis, renal calculus or stone, Staghorn calculus, ureteric stone, calculous pyelonephritis), calculus of lower urinary tract, bladder or urethra, renal colic), renal osteodystrophy, azotaemic osteodystrophy, nephrogenic diabetes insipidus, Lightwood-Albright syndrome, renal tubular acidosis, atrophy or hypertrophy of kidney, unilateral or bilateral Ischaemia and infarction of kidney, megaloureter, nephroptosis, pyelitis, pyeloureteritis, ureteritis, ureterocele, disorders of kidney and ureter in infectious and parasitic diseases (e.g., schistosomiasis, tuberculosis, syphilis), polycystic kidney disease, cystitis (e.g., acute cystitis, prostatocystitis, interstitial cystitis, irradiation cystitis, trigonitis, urethrotrigonitis) uninhibited, reflex or flaccid neuropathic bladder, neuromuscular dysfunction of bladder, neurogenic bladder dysfunction, overactive bladder, Bladder-neck obstruction (e.g., bladder-neck stenosis, vesicointestinal fistula, vesical fistula, diverticulum of bladder), bladder disorders in diseases (e.g., tuberculous cystitis, balder disorder in schistosomiasis), frequent micturition, polyuria, oliguria, anuria, nocturia, enuresis, dysuria, urinary incontinence, pneumaturia, disorders of urethra (e.g. urethritis and urethral syndrome, ulcer of urethra, urethral meatitis, urethral stricture, urethral, urethroperineal or urethrorectal fistula, urethral diverticulum, urethral caruncle, prolapsed urethral mucosa, urethritis and urethral disorders in other diseases such as candidal urethritis), urinary tract infection, urinary incontinence (overflow, reflex or urge), stress incontinence, diseases of the male genital organs (e.g., hyperplasia of prostate including adenofibromatous hypertrophy, adenoma, fibroadenoma, fibroma, hypertrophy, myoma), inflammatory diseases of prostate (e.g., acute or chronic prostatitis, abscesses of the prostate, prostatocystitis), calculus of prostate, congestion and haemorrhage of prostate, atrophy of prostate, hydrocele (e.g., hydrocele of spermatic cord, testis, or tunica vaginalis, encysted hydrocele, infected hydrocele, spermatocele), Torsion of testis, orchitis, epididymitis and epididymo-orhitis with or without abscess, redundant prepuce, phimosis and paraphimosis, leukoplakia of penis (e.g., Balanitis xerotica obliterans, Kraurosis pf penis), Balanoposthitis, priapism, ulcer of penis, induratio penis plastica (e.g., Peyrone's disese), atrophy, hypertrophy or thrombosis of corpus cavernosum and penis, inflammatory disorders of seminal vesicle (e.g., vesiculitis), of spermatic cord, of tunica vaginalis, of vas deferens, or of scrotum, atrophy of testis, vascular disorders of male genital organs (e.g., haematocele, haemorrhage, thrombosis), disorders of male genital organs in other diseases (e.g., gonococal, trichonal or tuberculous prostatitis, chlamydial, gonoccocal, or tuberculous epididymitis and/or orchitis, filarial chylocele, herpesviral infection of genital tract, tuberculosis of seminal vesicle), disorders of breast [e.g. benign mammary dysplasia, fibrocystic or diffuse mastopathy, cyst of the breast, fibroadenosis or fibrosclerosis of the breast, inflammatory disorders of the breast (e.g. abscess, carbuncle, acute, subacute or nonpurpuerperal mastitis), hypertrophy, lump in breast, fissure and fistula of nipple, fat necrosis and atrophy of the breast, galactorrhea, mastodynia, induration of breast, galactocele, inflammatory diseases of female pelvis organs (e.g., acute or chronic salpingitis and oophoritis), (abscess of fallopian tubes and/or ovary, pyosalpinx, salpingo-oophoritis, hydrosalpinx), acute and chronic inflammatory diseases of the uterus and cervix (e.g., endomyometritis, metritis, myometritis, pyometra, uterine abscess, cervicitis, endocervicitis, exocervicitis), acute and chronic parametritis and pelvic cellulites, acute and chronic pelvic peritonitis, female pelvic inflammatory disorders associated with other diseases (e.g., tuberculous infection of the cervix uteri, syphilitic, gonococcal and chlamydial pelvic inflammatory disorders), diseases of Bartholin's gland, inflammation of the vagina and vulva (e.g., acute, subacute and chronic vaginitis, acute, subacute and chronic vulvitis, vulvovaginitis as well as vaginitis, vulvitis and vulvovaginitis in infectious and parasitic diseases (eg., candidiasis, herpesviral infection and pinworm infection), ulceration of vagina and vulva (e.g., ulceration in hepesviral infection and tuberculosis), endometriosis (e.g., endometriosis of the uterus, ovary, fallopian tube, pelvic peritoneum, vagina and intestine), female genital prolapse, fistulae involving female genital tract, polyp (e.g., polyp of corpus uteri, cervix uteri, vagina and vulva), dysplasia of the cervix uteri, vagina and vulva, menstruation disorders (e.g., primary and secondary amenorrhea, oligomenorrhea, excessive menstruation, ovulation bleeding, menorrhagia), pain associated with female genital organs and menstrual cycle (e.g., Mittleschmerz, dyspareunia, vaginismus, primary and secondary dysmenorrheal and postmenopausal disorders), neoplasm of the genitourinary system (e.g., neoplasm of the kidney, urether, urethra, bladder, cervix, uterus, vagina, vulva, ovary, penis, prostate and testis)

Examples of diseases or disorders associated with neuropathic pain include, but are not limited to, neuralgia (e.g., posttherapeutic neuralgia, postherpetic neuralgia and trigeminal neuralgia), neuropathy (e.g., diabetic neuropathy), neuropathic pain, orofacial neuropathic pain, pain associated with cancer, psychogenic pain, headache (e.g., nonorganic chronic headache, tension-type headache, cluster headache and migraine), conditions associated with chronic cephalic pain, complex regional pain syndrome, nerve trunk pain, somatoform pain disorder, cyclical mastalgia, chronic fatigue syndrome, multiple somatization syndrome, chronic pain disorder, tabes dorsalis, spinal cord injury, central pain, noncardiac chest pain, central post-stroke pain, shingles, and Morton's neuroma.

Inflammatory disorders that may be treated according to the methods of the invention include, but are not limited to, inflammation associated with microbial infections (e.g., bacterial, viral and fungal infections), physical agents (e.g., burns, radiation, and trauma), chemical agents (e.g., toxins and caustic substances), tissue necrosis and various types of immunologic reactions. Examples of inflammatory disorders further include, but are not limited to, disorders of the musculoskeletal and connective tissue system, disorders of the respiratory system, disorders of the circulatory system, disorders of the genitourinary system and disorders of the gastrointestinal system. Inflammatory disorders of these systems include, but are not limited to those exemplified above. Exemplary inflammatory disorders include, but are not limited to arthritis (e.g., osteoarthritis, rheumatoid arthritis), acute and chronic infections (bacterial, viral and fungal); acute and chronic bronchitis, sinusitis, and other respiratory infections, including the common cold; acute and chronic asthma; acute and chronic gastroenteritis and colitis; acute and chronic cystitis and urethritis; acute respiratory distress syndrome; cystic fibrosis; acute and chronic dermatitis; acute and chronic conjunctivitis; acute and chronic serositis (pericarditis, peritonitis, synovitis, pleuritis and tendinitis); uremic pericarditis; acute and chronic cholecystis; acute and chronic vaginitis; acute and chronic uveitis; lupus erythematosus, eczema, shingles, psoriasis, hyperalgesia, irritable bowl syndrome, Crohn's disease, multiple sclerosis, drug reactions; and burns (thermal, chemical, and electrical).

Neurological disorders that may be treated according to the methods of the invention include, but are not limited to schizophrenia, bipolar disorder, depression, degenerative diseases such as Alzheimer's disease, epilepsy, musculoskeletal diseases, neuromuscular diseases (e.g., muscular dystrophy), multiple sclerosis, amyotrophic lateral sclerosis, stroke, addiction, cerebral ischemia, ventricular cardiac arrhythmia (e.g., sustained ventricular tachydardia, non-sustained ventricular tachycardia, ventricular fibrillation, ventricular premature beats and ventricular flutter), and atrial tachyarrhythmia (e.g., atrial fibrillation and atrial flutter), neuropathy (e.g., anticancer-agent-intoxicated neuropathy, diabetic neuropathy), retinal pigment degeneration, glaucoma, Huntington's chorea, Parkinson's disease and cancer of the nervous system (e.g., brain and spinal cord).

In one embodiment, the invention provides a method of treating a condition mediated by an NT/NTR, NGF/NTR, NGF/BDNF/NTR or BDNF/NTR interaction in a subject. The method comprises the step of administering to the subject a therapeutically effective amount of a NT/NTR, NGF/NTR, NGF/BDNF/NTR or BDNF/NTR modulator. The condition to be treated can be any condition which is mediated, at least in part, by interaction of a neurotrophin, e.g. NGF and/or BDNF, to the neurotrophin receptor.

The quantity of a given compound to be administered will be determined on an individual basis and will be determined, at least in part, by consideration of the individual's size, the severity of symptoms to be treated and the result sought. The NT/NTR modulators described herein can be administered alone or in a pharmaceutical composition comprising the modulator, an acceptable carrier or diluent and, optionally, one or more additional drugs.

The NT/NTR modulator can be administered subcutaneously, intravenously, parenterally, intraperitoneally, intradermally, intramuscularly, topically, enterally (e.g., orally), rectally, nasally, buccally, sublingually, vaginally, by inhalation spray, by drug pump or via an implanted reservoir in dosage formulations containing conventional non-toxic, physiologically acceptable carriers or vehicles. The preferred method of administration is by oral delivery. The form in which it is administered (e.g., syrup, elixir, capsule, tablet, solution, foams, emulsion, gel, sol) will depend in part on the route by which it is administered. For example, for mucosal (e.g., oral mucosa, rectal mucosa, intestinal mucosa, bronchial mucosa) administration, nose drops, aerosols, inhalants, nebulizers, eye drops or suppositories can be used. The compounds and agents of this invention can be administered together with other biologically active agents, such as analgesics, e.g., opiates, anti-inflammatory agents, e.g., NSAIDs, anesthetics and other agents which can control one or more symptoms or causes of a NTR-mediated condition.

In a specific embodiment, it may be desirable to administer the agents of the invention locally to a localized area in need of treatment; this may be achieved by, for example, and not by way of limitation, local infusion during surgery, topical application, transdermal patches, by injection, by means of a catheter, by means of a suppository, or by means of an implant, said implant being of a porous, non-porous, or gelatinous material, including membranes, such as sialastic membranes or fibers. For example, the agent can be injected into the joints or the urinary bladder.

The compounds of the invention can, optionally, be administered in combination with one or more additional drugs which, for example, are known for treating and/or alleviating symptoms of the condition mediated by BDNF, NGF p75^{NTR}, TrkA and TrkB. The additional drug can be administered simultaneously with the compound of the invention, or sequentially. For example, the compounds of the invention can be administered in combination with at least one of an analgesic, an anti-inflammatory agent, an anesthetic, a corticosteroid (e.g., beclomethasone diproprionate (BDP) treatment), an anti-convulsant, an antidepressant, an anti-nausea agent, an anti-psychiatric agent, a cardiovascular agent (e.g., a beta-blocker) or a cancer therapeutic. In certain embodiments, the compounds of the invention are administered in combination with a pain drug. As used herein the phrase, “pain drugs” is intended to refer to analgesics, anti-inflammatory agents, anesthetics, corticosteroids, antiepileptics, barbiturates, antidepressants, and marijuana.

As used herein, an “analgesic” is an agent that relieves pain without significant impairment of consciousness or sense perception and may result in the reduction of inflammation as do corticosteroids, e.g., an anti-inflammatory agent. Analgesics can be subdivided into NSAIDs (non-steroidal-anti-inflammatory agents) narcotic analgesics, and non-narcotic agents. NSAIDs can be further subdivided into non-selective COX (cyclooxygenase) inhibitors, and selective COX2 inhibitors. Opioid analgesics can be natural, synthetic or semi-synthetic opioid (narcotic) analgesics, and include for example, morphine, codeine, meperidine, propxyphen, oxycodone, hydromorphone, heroine, tranadol, and fentanyl. Non-opioid analgesics (non-narcotic) analgesics include, for example, acetaminophen, paracetamol, clonidine, NMDA antagonists, and cannabinoids. Non-selective COX inhibitors include, but are not limited to acetylsalicylic acid (ASA), ibuprofen, naproxen, ketoprofen, piroxicam, etodolac, and bromfenac. Selective COX2 inhibitors include, but are not limited to celecoxib, valdecoxib, parecoxib, and etoricoxib.

As used herein an “anesthetic” is an agent that interferes with sense perception near the site of administration, a local anesthetic, or result in alteration or loss of consciousness, e.g., systemic anesthetic agents. Local anesthetics include but are not limited to lidocaine and buvicaine.

Non-limiting examples of antiepileptic agents are carbamazepine, phenytoin and gabapentin. Non-limiting examples of antidepressants are amitriptyline and desmethylimiprimine.

Non-limiting examples of anti-inflammatory drugs include corticosteroids (e.g., hydrocortisone, cortisone, prednisone, prednisolone, methyl prednisone, triamcinolone, fluprednisolone, betamethasone and dexamethasone), salicylates, antihistamines and H₂ receptor antagonists.

The present invention provides a method for treating a subject that would benefit from administration of a composition of the present invention. Any therapeutic indication that would benefit from a NT/NTR modulator can be treated by the methods of the invention. The method includes the step of administering to the subject a composition of the invention, such that the disease or disorder is treated.

The above methods can be employed in the absence of other treatment, or in combination with other treatments. Such treatments can be started prior to, concurrent with, or after the administration of the compositions of the instant invention. Accordingly, the methods of the invention can further include the step of administering a second treatment, such as a second treatment for the disease or disorder or to ameliorate side effects of other treatments. Such second treatment can include, e.g., anti-inflammatory medication and any treatment directed toward treating pain. Additionally or alternatively, further treatment can include administration of drugs to further treat the disease or to treat a side effect of the disease or other treatments (e.g., anti-nausea drugs, anti-inflammatory drugs, anti-depressants, anti-psychiatric drugs, anti-convulsants, steroids, cardiovascular drugs, and cancer chemotheraputics).

The invention further provides a method for preventing in a subject, a disease or disorder which can be treated with administration of the compositions of the invention. Subjects “at risk” may or may not have detectable disease, and may or may not have displayed detectable disease prior to the treatment methods described herein. “At risk” denotes that an individual who is determined to be more likely to develop a symptom based on conventional risk assessment methods or has one or more risk factors that correlate with development of a disease or disorder that may be treated according the methods of the invention. For example, risk factors include family history, medication history, and history of exposure to an environmental substance which is known or suspected to increase the risk of disease. Subjects at risk for a disease or condition which can be treated with the agents mentioned herein can also be identified by, for example, any or a combination of diagnostic or prognostic assays known to those skilled in the art. Administration of a prophylactic agent can occur prior to the manifestation of symptoms characteristic of the disease or disorder, such that the disease or disorder is prevented or, alternatively, delayed in its progression.

### EXEMPLIFICATION OF THE INVENTION

The invention is further illustrated by the following examples, which should not be construed as further limiting. The animal models used throughout the Examples are accepted animal models and the demonstration of efficacy in these animal models is predictive of efficacy in humans.

### Example 1

### Materials and Methods

Cell Culture

Cells were incubated at 37° C. in 5% CO₂. PC12 and PC12^{nnr5} cells were maintained in RPMI-1640 medium supplemented with 10% fetal calf serum (FCS); cells were harvested by replacing the medium with calcium-magnesium-free balanced salt (Gey's) solution and incubating at 37° C. for 15 min. NIH-3T3 cells were grown in Dulbecco's modified Eagle medium: F12 containing 5% FCS; cells were harvested by replacing the medium with a 0.25% trypsin-0.53 mM ethylenediaminetetraacetic acid (EDTA) solution and incubating at 37° C. for 5 min.

Cell Transfections

The full-length TrkB cDNA was cloned into the CMV plasmid vector. Transient expression of TrkB in PC12^{nnr5} cells was achieved by transfecting the cells with the TrkB expression vector using Lipofectamine Plus reagents (Invitrogen Canada Inc; Burlington, ON). PC12^{nnr5} cells were passed through a syringe fitted with a 21-gauge needle several times to prevent clumping. In a 100 mm dish, 10×10⁶ cells were plated and used for transfection the next day. A solution of 12 μg DNA and 30 μL Plus reagent diluted in 750 μL OptiMEM medium (Invitrogen) was incubated at room temperature for 15 min. The mixture was then added to 60 μL Lipofectamine reagent in 750 μL OptiMEM medium and incubated for another 15 min at room temperature. Cells were rinsed once with serum-free RPMI-1640 and then placed in 5.0 mL of the medium. The DNA mixture was added directly to the plated cells, which were subsequently incubated at 37° C. After 3 h, 6.5 mL of RPMI-1640 containing 20% FCS was added to the dish, restoring serum levels to 10%. Cells were used 24 hours post-transfection.

Immunoprecipitation

Cell samples were solubilized in 1 mL lysis buffer (Tris-buffered saline [TBS; 10 mM Tris-HCl pH 8.0, 150 mM NaCl] containing 10% glycerol, 1% Triton X-100, 1 mM phenyl-methyl-sulphonylfluoride [PMSF], 10 μg/mL aprotinin, and 1 μg/mL leupeptin) and incubated for 30 min at 4° C. Samples were centrifuged to remove insolubilities, and either 18 μL rabbit polyclonal anti-Trk cytoplasmic domain antibody 545 or 2 μL rabbit polyclonal anti-p75^{NTR} antibody 9992 was added to the supernatant. Samples were incubated with rotation overnight at 4° C. To isolate antibody complexes, 70 μL of a 50% slurry of UltraLink Immobilized Protein G (Pierce Biotechnology Inc; Rockford, Ill.) in lysis buffer was incubated with the samples, rotating, for 2 h at 4° C. The solid phase was washed twice with cold lysis buffer, once with distilled water, and the isolated proteins were dissolved in sodium dodecyl sulfate (SDS) sample buffer (10 mM Tris-HCl pH 6.8 containing 5% glycerol, 5% β-mercaptoethanol, 3% SDS, and 0.5% bromophenol blue) and heated for 10 min at 95° C.

Neurotrophin-Receptor Crosslinking

PC12 cells or TrkB-expressing PC12^{nnr5} cells were recovered using Gey's solution, pelleted by centrifugation, and suspended in HKR (HEPES-Krebs Ringer Solution; [10 mM HEPES, 125 mM NaCl, 4.8 mM KCl, 1.3 mM CaCl₂.2H₂O, 1.2 mM MgSO₄, 1.2 mM KH₂PO₄, 1 mg/mL BSA, 1 mg/mL glucose, pH 7.35]). In a total volume of 1 mL, 1×10⁶ cells were incubated, rotating, with 0.1 nM ¹²⁵I-BDNF or ¹²⁵I-NGF and additions (Compound A or Compound B at the indicated final concentrations) for 2 h at 4° C. At the conclusion of the binding reaction, a 20 μL volume of BS³ was added to a final concentration of 0.4 mM and incubated, rocking, for an additional 30 min at room temperature. Cells were washed twice in TBS. For analysis of ¹²⁵I-NGF crosslinking to TrkA, ¹²⁵I-NGF crosslinking to p75^{NTR}, and ¹²⁵I-BDNF crosslinking to p75^{NTR}, samples were solubilized directly in SDS sample buffer and heated for 10 min at 95° C. For analysis of ¹²⁵I-BDNF crosslinking to TrkB, samples were immunoprecipitated with a pan-Trk antibody. All samples were electrophoresed on a 6% SDS-polyacrylamide gel, dried, and autoradiographed. Dried gels were registered to the autoradiograph and each labeled protein band was quantified by direct gamma counting. Non-linear regression analysis was performed by Prism 3.0 to generate concentration-effect curves and determine IC₅₀ values.

Trk Phosphorylation Assay

PC12 cells were used to assess TrkA phosphorylation; TrkB-expressing PC12^{nnr5} cells were used to assess TrkB phosphorylation with PC12^{nnr5} cells serving as a non-transfected control. Cells were recovered using Gey's solution and pelleted by centrifugation. In a total volume of 1 mL, 1×10⁶ cells were suspended in HKR and incubated with additions (Compound A or Compound B at the indicated final concentrations) in a total volume of 1 mL for 15 min at 37° C. Samples were washed once in cold phosphate-buffered saline (PBS; 150 mM NaCl pH 7.4 containing 1.7 mM KH₂PO₄ and 5.2 mM Na₂HPO₄) and once in cold TBS, then immunoprecipitated with a pan-Trk antibody using lysis buffer containing 500 μM orthovanadate. Samples were separated by electrophoresis on a 6% SDS-polyacrylamide gel and transferred to a nitrocellulose membrane for western blotting. Membranes were blocked with 2% BSA in Tris-buffered saline/Tween 20 (TBS-T; 10 mM Tris-HCl pH 8.0, 150 mM NaCl, 0.1% Tween) for 30 min at room temperature, followed by an overnight incubation in a 1:2000 dilution of the anti-phosphotyrosine antibody 4G10 (Upstate Biotechnology; Lake Placid, N.Y.) in TBS-T at 4° C. The membrane was washed several times in TBS-T, incubated for 30 min at room temperature with a horseradish peroxidase (HRP)-linked anti-mouse antibody diluted 1:3000 in the blocking solution, and washed several times more. HRP activity was visualized with enhanced chemiluminescence (Pierce). The resulting bands were quantified by densitometry. Prism 3.0 was used to make a single comparison between each treatment and the control using perform t tests.

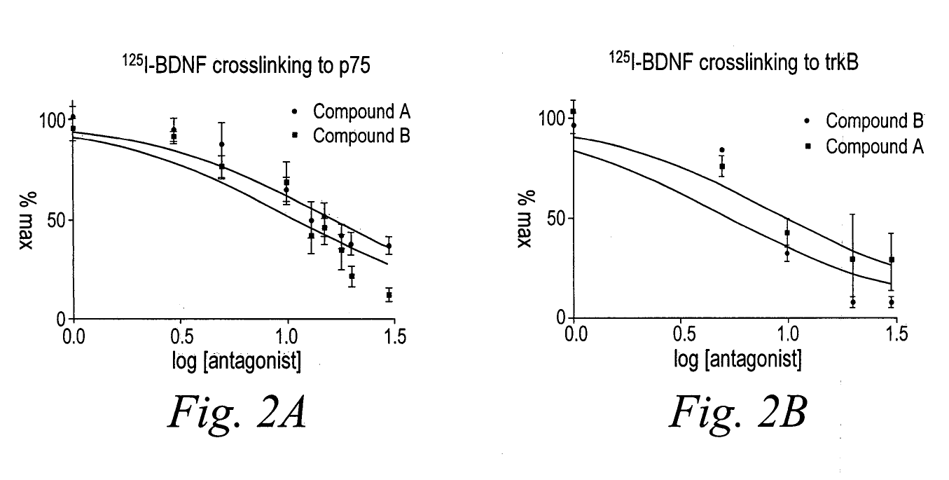

The data in FIGS. 1 and 2 demonstrate that Compound A inhibits the chemical crosslinking of ¹²⁵I-NGF to receptor p75^{NTR}, and ¹²⁵I-BDNF to its receptors p75^{NTR} and TrkB. Compound B inhibits the chemical crosslinking of ¹²⁵I-NGF to p75^{NTR} and TrkA, and ¹²⁵I-BDNF to p75^{NTR} and TrkB. For the p75^{NTR} receptor, Compound A has an IC₅₀ (mean±S.E.M) 5.82 μm±0.03 (n=11); Compound B has an IC₅₀ of 5.68±0.04 (n=10). At the TrkA receptor, Compound B shows an IC₅₀ of 8.9±0.07 (n=3). At p75^{NTR}, Compound A has an IC₅₀ 15.64±0.05 (n=5); Compound B has an IC₅₀ 10.56±0.05 (n=7). For the TrkB receptor, Compound A displays an IC₅₀ 9.60±0.11 (n=3); Compound B has an IC₅₀ 5.28±0.09 (n=4).

### Example 2

The compounds were screened for effects on binding of ¹²⁵I-BDNF or ¹²⁵I-NGF to PC12 cells (p75^{NTR} receptors), and those that inhibited binding were further tested to determine the EC₅₀ for this inhibition.

Materials

PC12 cells were cultured and prepared for binding assays as described in Example 1.

Lyophilized Serotec mouse 2.5S NGF (Cedarlane Laboratories, Toronto, Canada, cat. # PMP 04Z) was reconstituted under sterile conditions to 1 mg/mL (38 μM) in sterile 0.2% acetic acid, and 100 μL or 50 μL aliquots were stored frozen at −80° C.

Lypophilized Peprotech human recombinant BDNF (Cedarlane Laboratories, Toronto, Canada, cat. # PE 450-02-10) was reconstituted under sterile conditions to 3.5 μM in sterile HKR/1%BSA, and either used immediately or stored in aliquots of 20 μl at −80° C. Immediately prior to use, a 15 nM (30×) solution of ¹²⁵I-BDNF or ¹²⁵I-NGF was prepared in HKR buffer, and was used at 0.5 nM final concentration.

HKR was prepared and stored in aliquots at −20° C. (usually 45 mL in a 50 mL conical tube). For use in experimental procedures, HKR buffer was used promptly after thawing and kept cold.

Gey's Balanced Salt Solution, calcium-magnesium-free (CMF) was prepared as a one liter 10× stock, pH 7.4 (86.6 g, 3.7 g KCl, 1.2 g Na₂HPO₄, 2.27 g NaHCO₃, 0.3 g KH₂PO₄, 10 g glucose), filter-sterilized and stored under refrigeration. For experimental procedures, the 10× stock was diluted 1:10 with sterile distilled water prior to use.

Test Compounds were dissolved to 10 mM in DMSO, and those that were not initially soluble were warmed to 40-50° C. and vortexed. Those compounds that were still not in solution were reported as such. Compounds were then stored in this form at −20° C. until use. Immediately before experiments, 100 mM compounds were diluted to 10 mM in DMSO (1:10), followed by a further dilution to 1 mM (1:10) with distilled water, and a further dilution to 500 μM (1:1) with HKR.

Basic Screening Assay:

Assays to determine maximum binding (Bmax), non-specific binding (NSB), and binding in the presence of each test compound (e.g., x, y) were performed at least in duplicate. In a total volume of 300 μL, 4×10⁶ PC12 cells were incubated, rotating for 2 h at 4° C. with the following:

| | | ¹²⁵I-BDNF | | | | |
|---|---|---|---|---|---|---|
| | HKR | ¹²⁵I-NGF | | | | PC12 cells |
| | buffer | 15 nM | Cold NGF | Test Compound | Vehicle | 4 × 10⁶/mL |
| Bmax | 110 μL | 10 μL | — | — | 30 μL | 150 μL |
| NSB | 107 μL | 10 μL | 3 μL (400 nM) | — | 30 μL | 150 μL |
| x | 110 μL | 10 μL | — | 30 μL x | — | 150 μL |
| (50 μM) | | | | (500 μM) | | |
| y | 110 μL | 10 μL | — | 30 μL y | — | 150 μL |
| (50 μM) | | | | (500 μM) | | |

At the conclusion of the binding reaction, 100 μL aliquots of the reaction mixtures were carefully layered over 200 μL of a 10% glycerol solution in a 0.4 mL tube, and subjected to centrifugation in a microcentrifuge for 2 minutes at half maximum speed. The cell pellets containing the bound ¹²⁵I-BDNF or ¹²⁵I-NGF were then measured using a gamma counter. The mean values of the replicates were then calculated to determine the value for maximum binding (Bmax) and nonspecific binding (NSB), and binding in the presence of test compound at 50 μM (Bx). Specific binding (SB) was measured by subtracting the NSB from the Bmax. Specific binding in the presence of the test compound (SBₓ) was determined by subtracting the NSB from the Bx for each test compound; and each SBx was divided by SB and multiplied by 100 to give the specific binding in the presence of the compound as a percent of control specific binding.

Dose Response Binding Assay:

The EC₅₀ of test compounds (x) was determined by measuring serial dilutions as set forth below in the basic screening assay.

| | | | ¹²⁵I-BDNF | | | | |
|---|---|---|---|---|---|---|---|
| | | HKR | ¹²⁵I-NGF | Cold | | | PC12 cells |
| # | | buffer | 15 nM | NGF | Test Compound | Vehicle | 4 × 10⁶/mL |
| 1. | Bmax | 110 μL | 10 μL | — | — | 30 μL | 150 μL |
| 2. | NSB | 107 μL | 10 μL | 3 μL | — | 30 μL | 150 μL |
| 3. | x (50 μM) | 110 μL | 10 μL | — | 30 μL x (500 μM) | — | 150 μL |
| 4. | x (10 μM) | 110 μL | 10 μL | — | 30 μL x (100 μM) | — | 150 μL |
| 5. | x (5 μM) | 110 μL | 10 μL | — | 30 μL x (50 μM) | — | 150 μL |
| 6. | x (1 μM) | 110 μL | 10 μL | — | 30 μL x (10 μM) | — | 150 μL |
| 7. | x (0.5 μM) | 110 μL | 10 μL | — | 30 μL x (5 μM) | — | 150 μL |
| 8. | x (0.1 μM) | 110 μL | 10 μL | — | 30 μL x (1 μM) | — | 150 μL |

The data were then analyzed for each concentration of compound as a percent of control binding, and graphed to determine the EC₅₀ using Graphpad Prism software, version 3. The results for the compounds tested are set forth in Tables 1-4.

### Example 3

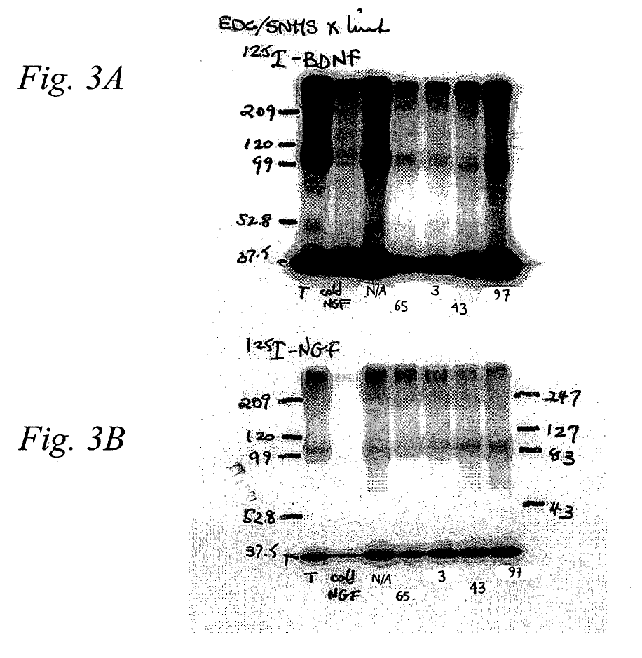

The neurotrophin-receptor crosslinking assay described in Example 1 was used to determine binding of compounds 65, 3, 43 and 97 to ¹²⁵I-NGF and ¹²⁵I-BDNF. Results of these experiments are shown in the table below and demonstrated in FIGS. 3A and 3B.

| | ¹²⁵I-NGF | | | ¹²⁵I-BDNF | | |
|---|---|---|---|---|---|---|
| | | X-linking | | | X-linking | |
| | Bind- | | p75 | Bind- | | p75 |
| Compound | ing | Sample¹ | band² | ing | Sample¹ | band² |
| Non-antag- | * | * | * | * | * | * |
| onist | | | | | | |
| (NA)³ | | | | | | |
| 65 | XXX | * | * | XXXXX | XXXXX | XXXXX |
| 3 | * | * | * | XXXXX | XXXXX | XXXXX |
| 43 | * | * | * | XXX | XXX | XXXXX |
| 97 | * | * | * | * | * | XX |

% BINDING KEY
0 < XXXXX < 10
10 < XXXX < 20
20 < XXX < 30
30 < XX < 40
40 < X < 50
50 < *

¹The p75 band was excised from the gel and cpm were determined on a gamma counter. Values were calculated as follows:

$$\frac{( cpm band ) - ( cpm cold NGF band )}{( cpm total xlink ) - ( cpm cold NGF band )}$$

²An aliquot of the total crosslinked sample in SDS-sample buffer was counted on a gamma counter. Values were calculated as follows:

$$\frac{( cpm sample ) - ( cpm cold NGF sample )}{( cpm total xlink ) - ( cpm cold NGF )}$$

³The non-antagonist is a non-binding compound that was used as a control.

Equivalents

Those skilled in the art will recognize, or be able to ascertain using no more than routine experimentation, many equivalents to the specific embodiments of the invention described herein. Such equivalents are intended to be encompassed by the following claims.

### INCORPORATION BY REFERENCE

The entire contents of all patents, published patent applications and other references cited herein are hereby expressly incorporated herein in their entireties by reference.

1. A method of modulating the interaction of a neurotrophin and a neurotrophin receptor, comprising contacting cells expressing a neurotrophin receptor with an effective amount of a compound of Formula 1, 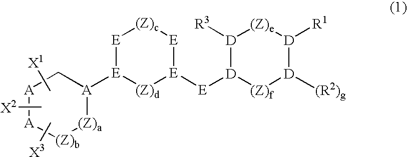
wherein
A, E and D are each, independently, an sp²- or sp³-hybridized oxygen, carbon, nitrogen, or sulfur atom;
X¹, X² and X³ are each, independently, selected from the group consisting of a hydrogen atom, C₁-C₆-alkyl, an electronegative atom, an electronegative functional group, and N(R⁴)R⁵, wherein R⁴ and R⁵ are each, independently, selected from the group consisting of H, aryl, and C₁-C₆-alkyl; esters thereof, salts thereof, and any combination thereof;
R¹, R² and R³ are each, independently, selected from the group consisting of a hydrogen atom, C₁-C₆-alkyl, an electronegative atom, and N(R⁴)R⁵, wherein R⁴ and R⁵ are each, independently, selected from the group consisting of H, aryl, and C₁-C₆-alkyl; esters thereof, salts thereof, and any combination thereof;
Z is independently selected from the group consisting of C═S, C, O, S, CH, C(O), N, NH, C-alkyl, N-alkyl, C-aryl, N-aryl, N-cycloalkyl and N—P, where P is a carbohydrate moiety;
a, b, c, d, e, f and g are each 0 or 1, provided that at least one of a and b is 1, at least one of c and d is 1, and at least one of e and f is 1.

2. The method of claim 1, wherein the compound of Formula 1 is a compound of Formula 2, 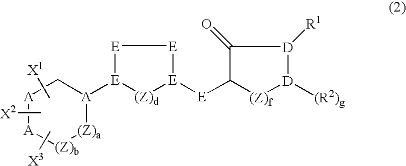
wherein
A, E and D are each, independently, an sp²- or sp³-hybridized carbon, nitrogen, oxygen or sulfur atom;
X¹, X² and X³ are each, independently, selected from the group consisting of a hydrogen atom, C₁-C₆-alkyl, an electronegative atom, an electronegative functional group, and N(R⁴)R⁵, where R⁴ and R⁵ are each, independently, H, aryl, and C₁-C₆-alkyl; esters thereof, salts thereof, and any combination thereof,
R¹ and R² are each, independently, selected from the group consisting of a hydrogen atom, C₁-C₆-alkyl, an electronegative atom, an electronegative functional group, and N(R⁴)R⁵, wherein R⁴ and R⁵ are each, independently, selected from the group consisting of H, aryl, and C₁-C₆-alkyl; esters thereof, salts thereof, and any combination thereof;
Z is independently selected from the group consisting of C═S, C, O, S, CH, C(O), N, NH, C-alkyl, N-alkyl, C-aryl, N-aryl, N-cycloalkyl and N—P;
a, b and g are each 0 or 1, provided that at least one of a and b is 1, and d and f are each 1.

3. The method of claim 1, wherein the compound of Formula 1 is a compound of Formula 3, 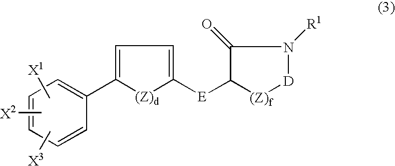
wherein
E and D are each, independently, an sp²- or sp³-hybridized carbon, nitrogen, oxygen or sulfur atom;
X¹, X² and X³ are each, independently, selected from the group consisting of a hydrogen atom, C₁-C₆-alkyl, an electronegative atom, an electronegative functional group and N(R⁴)R⁵, wherein R⁴ and R⁵ are each, independently, selected from the group consisting of H, aryl, and C₁-C₆-alkyl; esters thereof, salts thereof, and any combination thereof;
R¹ is selected from the group consisting of a hydrogen atom, C₁-C₆-alkyl, an electronegative atom, an electronegative functional group and N(R⁴)R⁵, wherein R⁴ and R⁵ are each, independently, selected from the group consisting of H, aryl, and C₁-C₆-alkyl; esters thereof, salts thereof, and any combination thereof;
Z is independently selected from the group consisting of C═S, C, O, S, CH, C(O), N, NH, C-alkyl, N-alkyl, C-aryl, N-aryl, N-cycloalkyl and N—P;
d and f are each 1.

4. The method of claim 1, wherein the compound of Formula 1 is a compound of Formula 4, 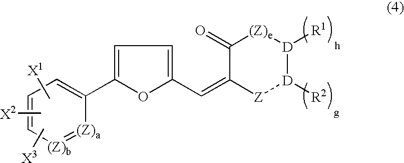
wherein
the dashed line indicates a double or single bond;
each D is, independently, an sp²- or sp³-hybridized carbon, nitrogen, oxygen or sulfur atom;
X¹, X² and X³ are each, independently, a hydrogen atom, C₁-C₆-alkyl, an electronegative atom or an electronegative functional group or N(R⁴)R⁵, wherein R⁴ and R⁵ are each, independently, H, aryl, C₁-C₆-alkyl, esters thereof, salts thereof, and any combination thereof;
R¹ and R² are each, independently, a hydrogen atom, C₁-C₆-alkyl, an electronegative atom or an electronegative functional group, or N(R⁴)R⁵, where R⁴ and R⁵ are each, independently, H, aryl, or C₁-C₆-alkyl; esters thereof, salts thereof, and any combination thereof,
Z is independently selected from the group consisting of C═S, C, O, S, CH, C(O), N, NH, C-alkyl, N-alkyl, C-aryl, N-aryl, N-cycloalkyl and N—P, wherein P is a carbohydrate moiety;
and a, b and e are each 0 or 1.

5-6. (canceled)

7. The method of claim 1, wherein the compound of Formula 1 is a compound of Formula 5, 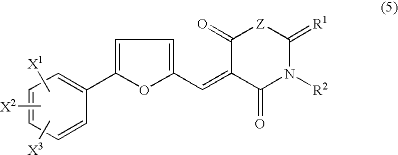
wherein
X¹, X² and X³ are each, independently, selected from the group consisting of a hydrogen atom, C₁-C₆-alkyl, an electronegative atom, an electronegative functional group, and N(R⁴)R⁵, wherein R⁴ and R⁵ are each, independently, H, aryl, C₁-C₆-alkyl; esters thereof, salts thereof, and any combination thereof;
R¹ and R² are each, independently, selected from the group consisting of a hydrogen atom, C₁-C₆-alkyl, an electronegative atom, an electronegative functional group, and N(R⁴)R⁵, where R⁴ and R⁵ are each, independently, H, aryl, or C₁-C₆-alkyl; esters thereof, salts thereof, and any combination thereof;
Z is independently selected from the group consisting of C═S, C, O, S, CH, C(O), N, NH, C-alkyl, N-alkyl, C-aryl, N-aryl, N-cycloalkyl and N—P, where P is a carbohydrate moiety.

8-9. (canceled)

10. The method of claim 1, wherein the compound of Formula 1 is a compound of Formula 6, 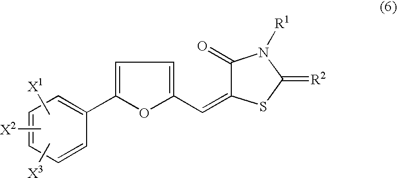
wherein
X¹, X² and X³ are each, independently, selected from the group consisting of a hydrogen atom, C₁-C₆-alkyl, an electronegative atom, an electronegative functional group, and N(R⁴)R⁵, wherein R⁴ and R⁵ are each, independently, selected from the group consisting of H, aryl, and C₁-C₆-alkyl; esters thereof, salts thereof, and any combination thereof;
R¹ and R² are each, independently, selected from the group consisting of a hydrogen atom, C₁-C₆-alkyl, an electronegative atom, an electronegative functional group and N(R⁴)R⁵, wherein R⁴ and R⁵ are each, independently, selected from the group consisting of H, aryl, and C₁-C₆-alkyl; esters thereof, salts thereof, and any combination thereof; and
Z is independently selected from the group consisting of C═S, C, O, S, CH, C(O), N, NH, C-alkyl, N-alkyl, C-aryl, N-aryl, N-cycloalkyl and N—P, wherein P is a carbohydrate moiety.

11-16. (canceled)

17. The method of claim 1, wherein the neurotrophin is selected from the group consisting of nerve growth factor, brain-derived neurotrophic factor, neurotrophin-3, neurotrophin-4/5 and precursors thereof.

18. The method of claim 1, wherein the neurotrophin receptor is selected from the group consisting of p75^{NTR}, TrkA, TrkB and TrkC.

19. The method of claim 17, wherein the neurotrophin is nerve growth factor or proNGF.

20. The method of claim 19, wherein the neurotrophin receptor is p75^{NTR}.

21. The method of claim 19, wherein the neurotrophin receptor is TrkA.

22. The method of claim 20, wherein the compound further modulates the interaction of NGF and/or proNGF with TrkA.

23. The method of claim 17, wherein the neurotrophin is brain-derived growth factor (BDNF) and/or proBDNF.

24. The method of claim 23, wherein the neurotrophin receptor is p75^{NTR}.

25. The method of claim 23, wherein the neurotrophin receptor is TrkB.

26. The method of claim 24, wherein the compound further modulates the interaction of BDNF and/or proBDNF with TrkB.

27. The method of claim 1, wherein the compound further modulates the interaction of a second neurotrophin with a neurotrophin receptor.

28. The method of claim 27, wherein the second neurotrophin is selected from the group consisting of nerve growth factor, brain-derived neurotrophic factor, neurotrophin-3, neurotrophin-4/5 and precursors thereof.

29. The method of claim 27, wherein the first neurotrophin is NGF and/or proNGF and the second neurotrophin is BDNF and/or proBDNF.

30. The method of claim 29, wherein the neurotrophin receptor is p75^{NTR}.

31. The method of claim 30, wherein the compound further modulates the interaction of NGF and/or proNGF with TrkA.

32. The method of claim 30, wherein the compound further modulates the interaction of BDNF and/or proBDNF with TrkB.

33. The method of claim 1, wherein the compound further modulates the interaction of the neurotrophin with TrkC.

34. The method of claim 1, wherein the method is used to modulate a neurotrophin-mediated activity in a subject in need thereof.

35. The method of claim 34, wherein the neurotrophin-mediated activity is associated with pain.

36. The method of claim 34, wherein the neurotrophin-mediated activity is associated with an inflammatory disorder.

37. The method of claim 34, wherein the neurotrophin-mediated activity is associated with a neurological disorder.

38-57. (canceled)

58. A method of treating pain in a subject in need thereof, comprising administering to the subject an effective amount of a compound of Formula 1, 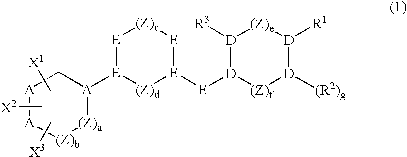
wherein
A, E and D are each, independently, an sp²- or sp³-hybridized oxygen, carbon, nitrogen, or sulfur atom;
X¹, X² and X³ are each, independently, selected from the group consisting of a hydrogen atom, C₁-C₆-alkyl, an electronegative atom, an electronegative functional group, and N(R⁴)R⁵, wherein R⁴ and R⁵ are each, independently, selected from the group consisting of H, aryl, and C₁-C₆-alkyl; esters thereof, salts thereof, and any combination thereof;
R¹, R² and R³ are each, independently, selected from the group consisting of a hydrogen atom, C₁-C₆-alkyl, an electronegative atom, and N(R⁴)R⁵, wherein R⁴ and R⁵ are each, independently, selected from the group consisting of H, aryl, and C₁-C₆-alkyl; esters thereof, salts thereof, and any combination thereof;
Z is independently selected from the group consisting of C═S, C, O, S, CH, C(O), N, NH, C-alkyl, N-alkyl, C-aryl, N-aryl, N-cycloalkyl and N—P, where P is a carbohydrate moiety;
a, b, c, d, e, f and g are each 0 or 1, provided that at least one of a and b is 1, at least one of c and d is 1, and at least one of e and f is 1.

59. The method of claim 58, wherein the compound of Formula 1 is a compound of Formula 2, 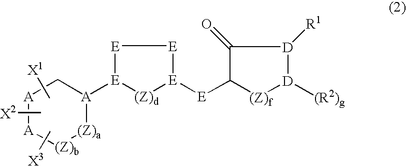
wherein
A, E and D are each, independently, an sp²- or sp³-hybridized carbon, nitrogen, oxygen or sulfur atom;
X¹, X² and X³ are each, independently, selected from the group consisting of a hydrogen atom, C₁-C₆-alkyl, an electronegative atom, an electronegative functional group, and N(R⁴)R⁵, where R⁴ and R⁵ are each, independently, H, aryl, and C₁-C₆-alkyl; esters thereof, salts thereof, and any combination thereof;
R¹ and R² are each, independently, selected from the group consisting of a hydrogen atom, C₁-C₆-alkyl, an electronegative atom, an electronegative functional group, and N(R⁴)R⁵, wherein R⁴ and R⁵ are each, independently, selected from the group consisting of H, aryl, and C₁-C₆-alkyl; esters thereof, salts thereof, and any combination thereof;
Z is independently selected from the group consisting of C═S, C, O, S, CH, C(O), N, NH, C-alkyl, N-alkyl, C-aryl, N-aryl, N-cycloalkyl and N—P;
a, b and g are each 0 or 1, provided that at least one of a and b is 1, and d and f are each 1.

60. The method of claim 58, wherein the compound of Formula 1 is a compound of Formula 3, 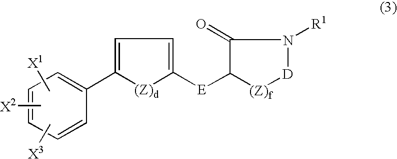
wherein
E and D are each, independently, an sp²- or sp³-hybridized carbon, nitrogen, oxygen or sulfur atom;
X¹, X²and X³ are each, independently, selected from the group consisting of a hydrogen atom, C₁-C₆-alkyl, an electronegative atom, an electronegative functional group and N(R⁴)R⁵, wherein R⁴ and R⁵ are each, independently, selected from the group consisting of H, aryl, and C₁-C₆-alkyl; esters thereof, salts thereof, and any combination thereof;
R¹ is selected from the group consisting of a hydrogen atom, C₁-C₆-alkyl, an electronegative atom, an electronegative functional group and N(R⁴)R⁵, wherein R⁴ and R⁵ are each, independently, selected from the group consisting of H, aryl, and C₁-C₆-alkyl; esters thereof, salts thereof, and any combination thereof;
Z is independently selected from the group consisting of C═S, C, O, S, CH, C(O), N, NH, C-alkyl, N-alkyl, C-aryl, N-aryl, N-cycloalkyl and N—P;
d and f are each 1.

61. The method of claim 58, wherein the compound of Formula 1 is a compound of Formula 4, 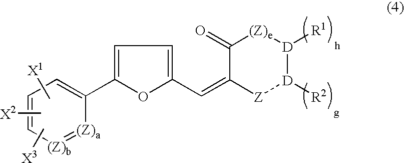
wherein
the dashed line indicates a double or single bond;
each D is, independently, an sp²- or sp³-hybridized carbon, nitrogen, oxygen or sulfur atom;
X¹, X² and X³ are each, independently, a hydrogen atom, C₁-C₆-alkyl, an electronegative atom or an electronegative functional group or N(R⁴)R⁵, wherein R⁴ and R⁵ are each, independently, H, aryl, C₁-C₆-alkyl, esters thereof, salts thereof, and any combination thereof;
R¹ and R² are each, independently, a hydrogen atom, C₁-C₆-alkyl, an electronegative atom or an electronegative functional group, or N(R⁴)R⁵, where R⁴ and R⁵ are each, independently, H, aryl, or C₁-C₆-alkyl; esters thereof, salts thereof, and any combination thereof;
Z is independently selected from the group consisting of C═S, C, O, S, CH, C(O), N, NH, C-alkyl, N-alkyl, C-aryl, N-aryl, N-cycloalkyl and N—P, wherein P is a carbohydrate moiety;
and a, b and e are each 0 or 1.

62-63. (canceled)

64. The method of claim 58, wherein the compound of Formula 1 is a compound of Formula 5, 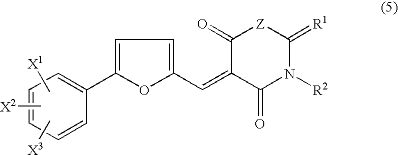
wherein
X¹, X² and X³ are each, independently, selected from the group consisting of a hydrogen atom, C₁-C₆-alkyl, an electronegative atom, an electronegative functional group, and N(R⁴)R⁵, wherein R⁴ and R⁵ are each, independently, H, aryl, C₁-C₆-alkyl; esters thereof, salts thereof, and any combination thereof;
R¹ and R² are each, independently, selected from the group consisting of a hydrogen atom, C₁-C₆-alkyl, an electronegative atom, an electronegative functional group, and N(R⁴)R⁵, where R⁴ and R⁵ are each, independently, H, aryl, or C₁-C₆-alkyl; esters thereof, salts thereof, and any combination thereof;
Z is independently selected from the group consisting of C═S, C, O, S, CH, C(O), N, NH, C-alkyl, N-alkyl, C-aryl, N-aryl, N-cycloalkyl and N—P, where P is a carbohydrate moiety.

65-66. (canceled)

67. The method of claim 58, wherein the compound of Formula 1 is a compound of Formula 6, 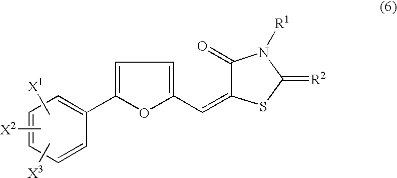
wherein
X¹, X² and X³ are each, independently, selected from the group consisting of a hydrogen atom, C₁-C₆-alkyl, an electronegative atom, an electronegative functional group, and N(R⁴)R⁵, wherein R⁴ and R⁵ are each, independently, selected from the group consisting of H, aryl, and C₁-C₆-alkyl; esters thereof, salts thereof, and any combination thereof;
R¹ and R² are each, independently, selected from the group consisting of a hydrogen atom, C₁-C₆-alkyl, an electronegative atom, an electronegative functional group and N(R⁴)R⁵, wherein R⁴ and R⁵ are each, independently, selected from the group consisting of H, aryl, and C₁-C₆-alkyl; esters thereof, salts thereof, and any combination thereof; and
Z is independently selected from the group consisting of C═S, C, O, S, CH, C(O), N, NH, C-alkyl, N-alkyl, C-aryl, N-aryl, N-cycloalkyl and N—P, wherein P is a carbohydrate moiety.

68-71. (canceled)

72. A method of treating an inflammatory disorder in a subject in need thereof, comprising administering to the subject and effective amount of a compound of Formula 1, 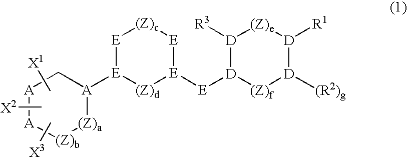
wherein
A, E and D are each, independently, an sp²- or sp³-hybridized oxygen, carbon, nitrogen, or sulfur atom;
X¹, X² and X³ are each, independently, selected from the group consisting of a hydrogen atom, C₁-C₆-alkyl, an electronegative atom, an electronegative functional group, and N(R⁴)R⁵, wherein R⁴ and R⁵ are each, independently, selected from the group consisting of H, aryl, and C₁-C₆-alkyl; esters thereof, salts thereof, and any combination thereof;
R¹, R² and R³ are each, independently, selected from the group consisting of a hydrogen atom, C₁-C₆-alkyl, an electronegative atom, and N(R⁴)R⁵, wherein R⁴ and R⁵ are each, independently, selected from the group consisting of H, aryl, and C₁-C₆-alkyl; esters thereof, salts thereof, and any combination thereof;
Z is independently selected from the group consisting of C═S, C, O, S, CH, C(O), N, NH, C-alkyl, N-alkyl, C-aryl, N-aryl, N-cycloalkyl and N—P, where P is a carbohydrate moiety;
a, b, c, d, e, f and g are each 0 or 1, provided that at least one of a and b is 1, at least one of c and d is 1, and at least one of e and f is 1.

73. The method of claim 72, wherein the compound of Formula 1 is a compound of Formula 2, 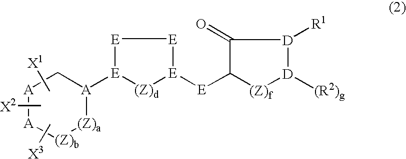
wherein
A, E and D are each, independently, an sp²- or sp³-hybridized carbon, nitrogen, oxygen or sulfur atom;
X¹, X² and X³ are each, independently, selected from the group consisting of a hydrogen atom, C₁-C₆-alkyl, an electronegative atom, an electronegative functional group, and N(R⁴)R⁵, where R⁴ and R⁵ are each, independently, H, aryl, and C₁-C₆-alkyl; esters thereof, salts thereof, and any combination thereof;
R¹ and R² are each, independently, selected from the group consisting of a hydrogen atom, C₁-C₆-alkyl, an electronegative atom, an electronegative functional group, and N(R⁴)R⁵, wherein R⁴ and R⁵ are each, independently, selected from the group consisting of H, aryl, and C₁-C₆-alkyl; esters thereof, salts thereof, and any combination thereof;
Z is independently selected from the group consisting of C═S, C, O, S, CH, C(O), N, NH, C-alkyl, N-alkyl, C-aryl, N-aryl, N-cycloalkyl and N—P;
a, b and g are each 0 or 1, provided that at least one of a and b is 1, and d and f are each 1.

74. The method of claim 72, wherein the compound of Formula 1 is a compound of Formula 3, 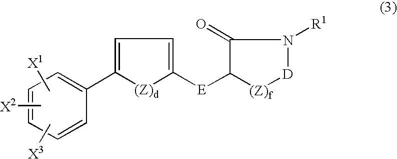
wherein
E and D are each, independently, an sp²- or sp³-hybridized carbon, nitrogen, oxygen or sulfur atom;
X¹, X² and X³ are each, independently, selected from the group consisting of a hydrogen atom, C₁-C₆-alkyl, an electronegative atom, an electronegative functional group and N(R⁴)R⁵, wherein R⁴ and R⁵ are each, independently, selected from the group consisting of H, aryl, and C₁-C₆-alkyl; esters thereof, salts thereof, and any combination thereof;
R¹ is selected from the group consisting of a hydrogen atom, C₁-C₆-alkyl, an electronegative atom, an electronegative functional group and N(R⁴)R⁵, wherein R⁴ and R⁵ are each, independently, selected from the group consisting of H, aryl, and C₁-C₆-alkyl; esters thereof, salts thereof, and any combination thereof;
Z is independently selected from the group consisting of C═S, C, O, S, CH, C(O), N, NH, C-alkyl, N-alkyl, C-aryl, N-aryl, N-cycloalkyl and N—P;
d and f are each 1.

75. The method of claim 72, wherein the compound of Formula 1 is a compound of Formula 4, 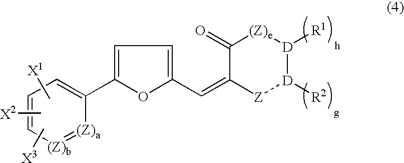
wherein
the dashed line indicates a double or single bond;
each D is, independently, an sp²- or sp³-hybridized carbon, nitrogen, oxygen or sulfur atom;
X¹, X² and X³ are each, independently, a hydrogen atom, C₁-C₆-alkyl, an electronegative atom or an electronegative functional group or N(R⁴)R⁵, wherein R⁴ and R⁵ are each, independently, H, aryl, C₁-C₆-alkyl, esters thereof, salts thereof, and any combination thereof;
R¹ and R² are each, independently, a hydrogen atom, C₁-C₆-alkyl, an electronegative atom or an electronegative functional group, or N(R⁴)R⁵, where R⁴ and R⁵ are each, independently, H, aryl, or C₁-C₆-alkyl; esters thereof, salts thereof, and any combination thereof;
Z is independently selected from the group consisting of C═S, C, O, S, CH, C(O), N, NH, C-alkyl, N-alkyl, C-aryl, N-aryl, N-cycloalkyl and N—P, wherein P is a carbohydrate moiety;
and a, b and e are each 0 or 1.

76-77. (canceled)

78. The method of claim 72, wherein the compound of Formula 1 is a compound of Formula 5, 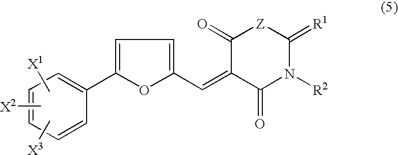
wherein
X¹, X² and X³ are each, independently, selected from the group consisting of a hydrogen atom, C₁-C₆-alkyl, an electronegative atom, an electronegative functional group, and N(R⁴)R⁵, wherein R⁴ and R⁵ are each, independently, H, aryl, C₁-C₆-alkyl; esters thereof, salts thereof, and any combination thereof;
R¹ and R² are each, independently, selected from the group consisting of a hydrogen atom, C₁-C₆-alkyl, an electronegative atom, an electronegative functional group, and N(R⁴)R⁵, where R⁴ and R⁵ are each, independently, H, aryl, or C₁-C₆-alkyl; esters thereof, salts thereof, and any combination thereof;
Z is independently selected from the group consisting of C═S, C, O, S, CH, C(O), N, NH, C-alkyl, N-alkyl, C-aryl, N-aryl, N-cycloalkyl and N—P, where P is a carbohydrate moiety.

79-80. (canceled)

81. The method of claim 72, wherein the compound of Formula 1 is a compound of Formula 6, 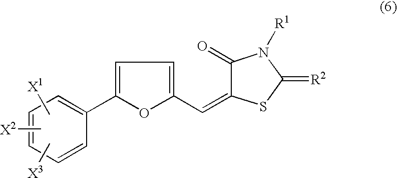
wherein
X¹, X² and X³ are each, independently, selected from the group consisting of a hydrogen atom, C₁-C₆-alkyl, an electronegative atom, an electronegative functional group, and N(R⁴)R⁵, wherein R⁴ and R⁵ are each, independently, selected from the group consisting of H, aryl, and C₁-C₆-alkyl; esters thereof, salts thereof, and any combination thereof;
R¹ and R² are each, independently, selected from the group consisting of a hydrogen atom, C₁-C₆-alkyl, an electronegative atom, an electronegative functional group and N(R⁴)R⁵, wherein R⁴ and R⁵ are each, independently, selected from the group consisting of H, aryl, and C₁-C₆-alkyl; esters thereof, salts thereof, and any combination thereof; and
Z is independently selected from the group consisting of C═S, C, O, S, CH, C(O), N, NH, C-alkyl, N-alkyl, C-aryl, N-aryl, N-cycloalkyl and N—P, wherein P is a carbohydrate moiety.

82-84. (canceled)

85. A method of treating a neurological disorder in a subject in need thereof, comprising administering an effective amount of a compound of Formula 1, 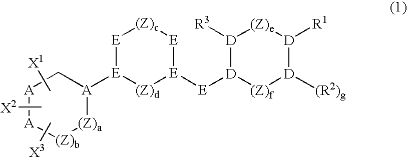
wherein
A, E and D are each, independently, an sp²- or sp³-hybridized oxygen, carbon, nitrogen, or sulfur atom;
X¹, X² and X³ are each, independently, selected from the group consisting of a hydrogen atom, C₁-C₆-alkyl, an electronegative atom, an electronegative functional group, and N(R⁴)R⁵, wherein R⁴ and R⁵ are each, independently, selected from the group consisting of H, aryl, and C₁-C₆-alkyl; esters thereof, salts thereof, and any combination thereof;
R¹, R² and R³ are each, independently, selected from the group consisting of a hydrogen atom, C₁-C₆-alkyl, an electronegative atom, and N(R⁴)R⁵, wherein R⁴ and R⁵ are each, independently, selected from the group consisting of H, aryl, and C₁-C₆-alkyl; esters thereof, salts thereof, and any combination thereof;
Z is independently selected from the group consisting of C═S, C, O, S, CH, C(O), N, NH, C-alkyl, N-alkyl, C-aryl, N-aryl, N-cycloalkyl and N—P, where P is a carbohydrate moiety;
a, b, c, d, e, f and g are each 0 or 1, provided that at least one of a and b is 1, at least one of c and d is 1, and at least one of e and f is 1.

86. The method of claim 85, wherein the compound of Formula 1 is a compound of Formula 2, 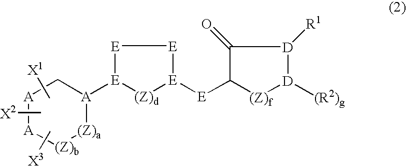
wherein
A, E and D are each, independently, an sp²- or sp³-hybridized carbon, nitrogen, oxygen or sulfur atom;
X¹, X² and X³ are each, independently, selected from the group consisting of a hydrogen atom, C₁-C₆-alkyl, an electronegative atom, an electronegative functional group, and N(R⁴)R⁵, where R⁴ and R⁵ are each, independently, H, aryl, and C₁-C₆-alkyl; esters thereof, salts thereof, and any combination thereof;
R¹ and R² are each, independently, selected from the group consisting of a hydrogen atom, C₁-C₆-alkyl, an electronegative atom, an electronegative functional group, and N(R⁴)R⁵, wherein R⁴ and R⁵ are each, independently, selected from the group consisting of H, aryl, and C₁-C₆-alkyl; esters thereof, salts thereof, and any combination thereof;
Z is independently selected from the group consisting of C═S, C, O, S, CH, C(O), N, NH, C-alkyl, N-alkyl, C-aryl, N-aryl, N-cycloalkyl and N—P;
a, b and g are each 0 or 1, provided that at least one of a and b is 1, and d and f are each 1.

87. The method of claim 85, wherein the compound of Formula 1 is a compound of Formula 3, 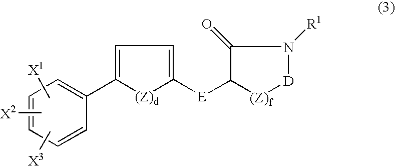
wherein
E and D are each, independently, an sp²- or sp³-hybridized carbon, nitrogen, oxygen or sulfur atom;
X¹, X² and X³ are each, independently, selected from the group consisting of a hydrogen atom, C₁-C₆-alkyl, an electronegative atom, an electronegative functional group and N(R⁴)R⁵, wherein R⁴ and R⁵ are each, independently, selected from the group consisting of H, aryl, and C₁-C₆-alkyl; esters thereof, salts thereof, and any combination thereof;
R¹ is selected from the group consisting of a hydrogen atom, C₁-C₆-alkyl, an electronegative atom, an electronegative functional group and N(R⁴)R⁵, wherein R⁴ and R⁵ are each, independently, selected from the group consisting of H, aryl, and C₁-C₆-alkyl; esters thereof, salts thereof, and any combination thereof,
Z is independently selected from the group consisting of C═S, C, O, S, CH, C(O), N, NH, C-alkyl, N-alkyl, C-aryl, N-aryl, N-cycloalkyl and N—P;
d and f are each 1.

88. The method of claim 85, wherein the compound of Formula 1 is a compound of Formula 4, 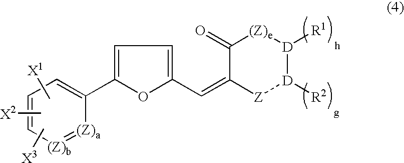
wherein
the dashed line indicates a double or single bond;
each D is, independently, an sp²- or sp³-hybridized carbon, nitrogen, oxygen or sulfur atom;
X¹, X² and X³ are each, independently, a hydrogen atom, C₁-C₆-alkyl, an electronegative atom or an electronegative functional group, or N(R⁴)R⁵, wherein R⁴ and R⁵ are each, independently, H, aryl, C₁-C₆-alkyl; esters thereof, salts thereof, and any combination thereof,
R¹ and R² are each, independently, a hydrogen atom, C₁-C₆-alkyl, an electronegative atom or an electronegative functional group, or N(R⁴)R⁵, where R⁴ and R⁵ are each, independently, H, aryl, or C₁-C₆-alkyl; esters thereof, salts thereof, and any combination thereof;
Z is independently selected from the group consisting of C═S, C, O, S, CH, C(O), N, NH, C-alkyl, N-alkyl, C-aryl, N-aryl, N-cycloalkyl and N—P, wherein P is a carbohydrate moiety;
and a, b and e are each 0 or 1.

89-90. (canceled)

91. The method of claim 85, wherein the compound of Formula 1 is a compound of Formula 5, 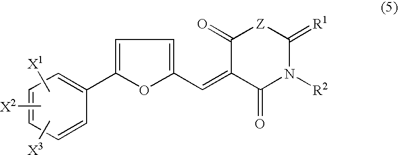
wherein
X¹, X² and X³ are each, independently, selected from the group consisting of a hydrogen atom, C₁-C₆-alkyl, an electronegative atom, an electronegative functional group, and N(R⁴)R⁵, wherein R⁴ and R⁵ are each, independently, H, aryl, C₁-C₆-alkyl; esters thereof, salts thereof, and any combination thereof;
R¹ and R² are each, independently, selected from the group consisting of a hydrogen atom, C₁-C₆-alkyl, an electronegative atom, an electronegative functional group, and N(R⁴)R⁵, where R⁴ and R⁵ are each, independently, H, aryl, or C₁-C₆-alkyl; esters thereof, salts thereof, and any combination thereof;
Z is independently selected from the group consisting of C═S, C, O, S, CH, C(O), N, NH, C-alkyl, N-alkyl, C-aryl, N-aryl, N-cycloalkyl and N—P, where P is a carbohydrate moiety.

92-93. (canceled)

94. The method of claim 85, wherein the compound of Formula 1 is a compound of Formula 6, 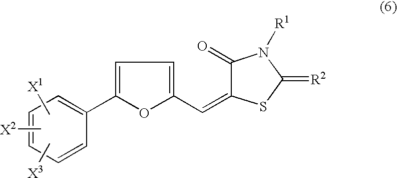
wherein
X¹, X² and X³ are each, independently, selected from the group consisting of a hydrogen atom, C₁-C₆-alkyl, an electronegative atom, an electronegative functional group, and N(R⁴)R⁵, wherein R⁴ and R⁵ are each, independently, selected from the group consisting of H, aryl, and C₁-C₆-alkyl; esters thereof, salts thereof, and any combination thereof;
R¹ and R² are each, independently, selected from the group consisting of a hydrogen atom, C₁-C₆-alkyl, an electronegative atom, an electronegative functional group and N(R⁴)R⁵, wherein R⁴ and R⁵ are each, independently, selected from the group consisting of H, aryl, and C₁-C₆-alkyl; esters thereof, salts thereof, and any combination thereof; and
Z is independently selected from the group consisting of C═S, C, O, S, CH, C(O), N, NH, C-alkyl, N-alkyl, C-aryl, N-aryl, N-cycloalkyl and N—P, wherein P is a carbohydrate moiety.

95-99. (canceled)

100. A pharmaceutical composition comprising a compound of Formula 1, 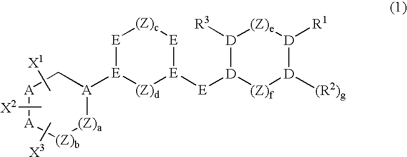
wherein
A, E and D are each, independently, an sp²- or sp³-hybridized oxygen, carbon, nitrogen, or sulfur atom;
X¹, X² and X³ are each, independently, selected from the group consisting of a hydrogen atom, C₁-C₆-alkyl, an electronegative atom, an electronegative functional group, and N(R⁴)R⁵, wherein R⁴ and R⁵ are each, independently, selected from the group consisting of H, aryl, and C₁-C₆-alkyl; esters thereof, salts thereof, and any combination thereof;
R¹, R² and R³ are each, independently, selected from the group consisting of a hydrogen atom, C₁-C₆-alkyl, an electronegative atom, and N(R⁴)R⁵, wherein R⁴ and R⁵ are each, independently, selected from the group consisting of H, aryl, and C₁-C₆-alkyl; esters thereof, salts thereof, and any combination thereof;
Z is independently selected from the group consisting of C═S, C, O, S, CH, C(O), N, NH, C-alkyl, N-alkyl, C-aryl, N-aryl, N-cycloalkyl and N—P, where P is a carbohydrate moiety;
a, b, c, d, e, f and g are each 0 or 1, provided that at least one of a and b is 1, at least one of c and d is 1, and at least one of e and f is 1.

101. The pharmaceutical composition of claim 100, comprising a compound of Formula 2, 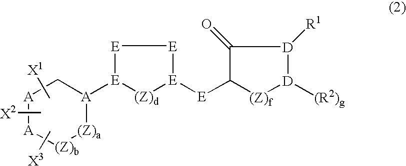
wherein
A, E and D are each, independently, an sp²- or sp³-hybridized carbon, nitrogen, oxygen or sulfur atom;
X¹, X² and X³ are each, independently, selected from the group consisting of a hydrogen atom, C₁-C₆-alkyl, an electronegative atom, an electronegative functional group, and N(R⁴)R⁵, where R⁴ and R⁵ are each, independently, H, aryl, and C₁-C₆-alkyl; esters thereof, salts thereof, and any combination thereof;
R¹ and R² are each, independently, selected from the group consisting of a hydrogen atom, C₁-C₆-alkyl, an electronegative atom, an electronegative functional group, and N(R⁴)R⁵, wherein R⁴ and R⁵ are each, independently, selected from the group consisting of H, aryl, and C₁-C₆-alkyl; esters thereof, salts thereof, and any combination thereof;
Z is independently selected from the group consisting of C═S, C, O, S, CH, C(O), N, NH, C-alkyl, N-alkyl, C-aryl, N-aryl, N-cycloalkyl and N—P;
a, b and g are each 0 or 1, provided that at least one of a and b is 1, and d and f are each 1.

102. (canceled)

103. The pharmaceutical composition of claim 100, comprising a compound of Formula 3, 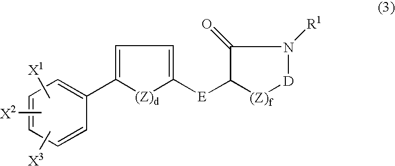
wherein
E and D are each, independently, an sp²- or sp³-hybridized carbon, nitrogen, oxygen or sulfur atom;
X¹, X² and X³ are each, independently, selected from the group consisting of a hydrogen atom, C₁-C₆-alkyl, an electronegative atom, an electronegative functional group and N(R⁴)R⁵, wherein R⁴ and R⁵ are each, independently, selected from the group consisting of H, aryl, and C₁-C₆-alkyl; esters thereof, salts thereof, and any combination thereof;
R¹ is selected from the group consisting of a hydrogen atom, C₁-C₆-alkyl, an electronegative atom, an electronegative functional group and N(R⁴)R⁵, wherein R⁴ and R⁵ are each, independently, selected from the group consisting of H, aryl, and C₁-C₆-alkyl; esters thereof, salts thereof, and any combination thereof;
Z is independently selected from the group consisting of C═S, C, O, S, CH, C(O), N, NH, C-alkyl, N-alkyl, C-aryl, N-aryl, N-cycloalkyl and N—P;
d and f are each 1.

104. The pharmaceutical composition of claim 100, comprising a compound of Formula 4, 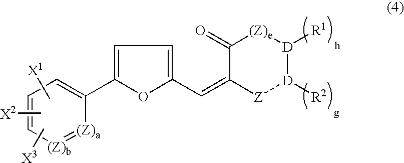
wherein
the dashed line indicates a double or single bond;
each D is, independently, an sp²- or sp³-hybridized carbon, nitrogen, oxygen or sulfur atom;
X¹, X² and X³ are each, independently, a hydrogen atom, C₁-C₆-alkyl, an electronegative atom or an electronegative functional group or N(R⁴)R⁵, wherein R⁴ and R⁵ are each, independently, H, aryl, C₁-C₆-alkyl, esters thereof, salts thereof, and any combination thereof;
R¹ and R² are each, independently, a hydrogen atom, C₁-C₆-alkyl, an electronegative atom or an electronegative functional group, or N(R⁴)R⁵, where R⁴ and R⁵ are each, independently, H, aryl, or C₁-C₆-alkyl; esters thereof, salts thereof, and any combination thereof;
Z is independently selected from the group consisting of C═S, C, O, S, CH, C(O), N, NH, C-alkyl, N-alkyl, C-aryl, N-aryl, N-cycloalkyl and N—P, wherein P is a carbohydrate moiety;
and a, b and e are each 0 or 1.

105-106. (canceled)

107. The pharmaceutical composition of claim 100, comprising a compound of Formula 5, 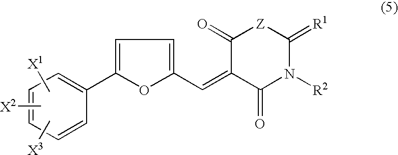
wherein
X¹, X² and X³ are each, independently, selected from the group consisting of a hydrogen atom, C₁-C₆-alkyl, an electronegative atom, an electronegative functional group, and N(R⁴)R⁵, wherein R⁴ and R⁵ are each, independently, H, aryl, C₁-C₆-alkyl; esters thereof, salts thereof, and any combination thereof;
R¹ and R² are each, independently, selected from the group consisting of a hydrogen atom, C₁-C₆-alkyl, an electronegative atom, an electronegative functional group, and N(R⁴)R⁵, where R⁴ and R⁵ are each, independently, H, aryl, or C₁-C₆-alkyl; esters thereof, salts thereof, and any combination thereof;
Z is independently selected from the group consisting of C═S, C, O, S, CH, C(O), N, NH, C-alkyl, N-alkyl, C-aryl, N-aryl, N-cycloalkyl and N—P, where P is a carbohydrate moiety.

108-109. (canceled)

110. The pharmaceutical composition of claim 100, comprising a compound of Formula 6, 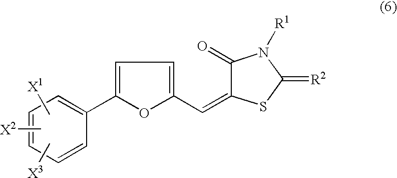
wherein
X¹, X² and X³ are each, independently, selected from the group consisting of a hydrogen atom, C₁-C₆-alkyl, an electronegative atom, an electronegative functional group, and N(R⁴)R⁵, wherein R⁴ and R⁵ are each, independently, selected from the group consisting of H, aryl, and C₁-C₆-alkyl; esters thereof, salts thereof, and any combination thereof;
R¹ and R² are each, independently, selected from the group consisting of a hydrogen atom, C₁-C₆-alkyl, an electronegative atom, an electronegative functional group and N(R⁴)R⁵, wherein R⁴ and R⁵ are each, independently, selected from the group consisting of H, aryl, and C₁-C₆-alkyl; esters thereof, salts thereof, and any combination thereof; and
Z is independently selected from the group consisting of C═S, C, O, S, CH, C(O), N, NH, C-alkyl, N-alkyl, C-aryl, N-aryl, N-cycloalkyl and N—P, wherein P is a carbohydrate moiety.

111-112. (canceled)

113. The method of claim 58, further comprising administering to the subject an additional therapeutic agent.

114. The method of claim 72, further comprising administering to the subject an additional therapeutic agent.

115. The method of claim 85, further comprising administering to the subject an additional therapeutic agent.
